SEQUENCE LISTING (308 sequences)

SEQ ID NO: 1 - DNA, 375 bp, synthetic construct
  misc_feature 1..375: note = Synthetic
  source 1..375: mol_type = other DNA
caggtccagttggtacagtctggggctgacgtgaagaagcctggggcctcagtgaaggtctcctgcaaggtttccggacatatcctcactgatttatccatgcactgggtgcgacagcctcctggaaaaggacttgagtggatggcaggttttgatcctgaagaaggtaaaataatctacgcacagaagttccagggcagagtcaccatgaccgaggacacatctacagacacagcctacatggagctgagcagcctgagatctggggacacggccgtttattactgtgcaacaagcgatattttgactgggtattatagagactactacggtttggacgtctggggccaagggaccacgctcaccgtctcctca

SEQ ID NO: 2 - protein, 125 aa, synthetic construct
  REGION 1..125: note = Synthetic
QVQLVQSGADVKKPGASVKVSCKVSGHILTDLSMHWVRQPPGKGLEWMAGFDPEEGKIIYAQKFQGRVTMTEDTSTDTAYMELSSLRSGDTAVYYCATSDILTGYYRDYYGLDVWGQGTTLTVSS

SEQ ID NO: 3 - DNA, 24 bp, synthetic construct
  misc_feature 1..24: note = Synthetic
  source 1..24: mol_type = other DNA
ggacatatcctcactgatttatcc

SEQ ID NO: 4 - protein, 8 aa, synthetic construct
  REGION 1..8: note = Synthetic
GHILTDLS

SEQ ID NO: 5 - DNA, 24 bp, synthetic construct
  misc_feature 1..24: note = Synthetic
  source 1..24: mol_type = other DNA
tttgatcctgaagaaggtaaaata

SEQ ID NO: 6 - protein, 8 aa, synthetic construct
  REGION 1..8: note = Synthetic
FDPEEGKI

SEQ ID NO: 7 - DNA, 54 bp, synthetic construct
  misc_feature 1..54: note = Synthetic
  source 1..54: mol_type = other DNA
gcaacaagcgatattttgactgggtattatagagactactacggtttggacgtc

SEQ ID NO: 8 - protein, 18 aa, synthetic construct
  REGION 1..18: note = Synthetic
ATSDILTGYYRDYYGLDV

SEQ ID NO: 9 - DNA, 336 bp, synthetic construct
  misc_feature 1..336: note = Synthetic
  source 1..336: mol_type = other DNA
gatattgtgatgactcagtctccactcttcctgcccgtcacccctggagagccggcctccatctcctgcaggtctagtcagagcctcctgcatagtaaaggatacaactatttggattggtacctgcagaagccagggcagtctccacaactcctgatctatttgggttctaatcgggcctccggggtccctgacaggttcagtggcagtggatcaggcacagattttacactgaaaatcagcagagtggaggctgaagatgttggggtttattactgcatgcaaactctacaaactcctcggacgttcggccaagggaccaaggtggaaatcaaa

SEQ ID NO: 10 - protein, 112 aa, synthetic construct
  REGION 1..112: note = Synthetic
DIVMTQSPLFLPVTPGEPASISCRSSQSLLHSKGYNYLDWYLQKPGQSPQLLIYLGSNRASGVPDRFSGSGSGTDFTLKISRVEAEDVGVYYCMQTLQTPRTFGQGTKVEIK

SEQ ID NO: 11 - DNA, 33 bp, synthetic construct
  misc_feature 1..33: note = Synthetic
  source 1..33: mol_type = other DNA
cagagcctcctgcatagtaaaggatacaactat

SEQ ID NO: 12 - protein, 11 aa, synthetic construct
  REGION 1..11: note = Synthetic
QSLLHSKGYNY

SEQ ID NO: 13-14 - skipped (2 entries)

SEQ ID NO: 15 - DNA, 27 bp, synthetic construct
  misc_feature 1..27: note = Synthetic
  source 1..27: mol_type = other DNA
atgcaaactctacaaactcctcggacg

SEQ ID NO: 16 - protein, 9 aa, synthetic construct
  REGION 1..9: note = Synthetic
MQTLQTPRT

SEQ ID NO: 17 - DNA, 375 bp, synthetic construct
  misc_feature 1..375: note = Synthetic
  source 1..375: mol_type = other DNA
caggtccagttggtacagtctggggctgacgtgaagaagcctggggcctcagtgaaggtctcctgcaaggtttccggacatatcctcactgatttatccatgcactgggtgcgacaggctcctggaaaagggcttgagtggatgggaggttttgatcctgaagaaggtgaaataatctacgcacagaagttccagggcagagtcaccatgaccgaggacacatctacagacacagcctacatggagctgagcagcctgagatctggggacacggccgtttattactgtgcaacaagcgatattttgactggttattatagagactactacggtttggacgtctggggccaagggaccacgctcaccgtctcctca

SEQ ID NO: 18 - protein, 125 aa, synthetic construct
  REGION 1..125: note = Synthetic
QVQLVQSGADVKKPGASVKVSCKVSGHILTDLSMHWVRQAPGKGLEWMGGFDPEEGEIIYAQKFQGRVTMTEDTSTDTAYMELSSLRSGDTAVYYCATSDILTGYYRDYYGLDVWGQGTTLTVSS

SEQ ID NO: 19 - DNA, 24 bp, synthetic construct
  misc_feature 1..24: note = Synthetic
  source 1..24: mol_type = other DNA
ggacatatcctcactgatttatcc

SEQ ID NO: 20 - protein, 8 aa, synthetic construct
  REGION 1..8: note = Synthetic
GHILTDLS

SEQ ID NO: 21 - DNA, 24 bp, synthetic construct
  misc_feature 1..24: note = Synthetic
  source 1..24: mol_type = other DNA
tttgatcctgaagaaggtgaaata

SEQ ID NO: 22 - protein, 8 aa, synthetic construct
  REGION 1..8: note = Synthetic
FDPEEGEI

SEQ ID NO: 23 - DNA, 54 bp, synthetic construct
  misc_feature 1..54: note = Synthetic
  source 1..54: mol_type = other DNA
gcaacaagcgatattttgactggttattatagagactactacggtttggacgtc

SEQ ID NO: 24 - protein, 18 aa, synthetic construct
  REGION 1..18: note = Synthetic
ATSDILTGYYRDYYGLDV

SEQ ID NO: 25 - DNA, 336 bp, synthetic construct; identical to SEQ ID NO: 9
  misc_feature 1..336: note = Synthetic
  source 1..336: mol_type = other DNA

SEQ ID NO: 26 - protein, 112 aa, synthetic construct; identical to SEQ ID NO: 10
  REGION 1..112: note = Synthetic

SEQ ID NO: 27 - DNA, 33 bp, synthetic construct; identical to SEQ ID NO: 11
  misc_feature 1..33: note = Synthetic
  source 1..33: mol_type = other DNA

SEQ ID NO: 28 - protein, 11 aa, synthetic construct
  REGION 1..11: note = Synthetic
QSLLHSKGYNY

SEQ ID NO: 29-30 - skipped (2 entries)

SEQ ID NO: 31 - DNA, 27 bp, synthetic construct
  misc_feature 1..27: note = Synthetic
  source 1..27: mol_type = other DNA
atgcaaactctacaaactcctcggacg

SEQ ID NO: 32 - protein, 9 aa, synthetic construct
  REGION 1..9: note = Synthetic
MQTLQTPRT

SEQ ID NO: 33 - DNA, 372 bp, synthetic construct
  misc_feature 1..372: note = Synthetic
  source 1..372: mol_type = other DNA
gaggagcaactggtggagtctgggggagacttggtacagcctggagggtccctaagactctcctgtgcagcctctggattcactctcagtagttatgaaatgaactgggtccgccaggctccagggaaggggctggagtgggtttcatacattagtagaggtggtagtctgatacactacacagactctgtgaagggccgattcaccatctccagagacaacgccaagaattcactgtatctgcaaatgaacagcctgagagccgaggacacggctgtttattactgtgtgagagacccagcagctcgttatcattattattatcacggtatggacgtctggggccaagggaccacggtcaccgtctcctca

SEQ ID NO: 34 - protein, 124 aa, synthetic construct
  REGION 1..124: note = Synthetic
EEQLVESGGDLVQPGGSLRLSCAASGFTLSSYEMNWVRQAPGKGLEWVSYISRGGSLIHYTDSVKGRFTISRDNAKNSLYLQMNSLRAEDTAVYYCVRDPAARYHYYYHGMDVWGQGTTVTVSS

SEQ ID NO: 35 - DNA, 24 bp, synthetic construct
  misc_feature 1..24: note = Synthetic
  source 1..24: mol_type = other DNA
ggattcactctcagtagttatgaa

SEQ ID NO: 36 - protein, 8 aa, synthetic construct
  REGION 1..8: note = Synthetic
GFTLSSYE

SEQ ID NO: 37 - DNA, 24 bp, synthetic construct
  misc_feature 1..24: note = Synthetic
  source 1..24: mol_type = other DNA
attagtagaggtggtagtctgata

SEQ ID NO: 38 - protein, 8 aa, synthetic construct
  REGION 1..8: note = Synthetic
ISRGGSLI

SEQ ID NO: 39 - DNA, 51 bp, synthetic construct
  misc_feature 1..51: note = Synthetic
  source 1..51: mol_type = other DNA
gtgagagacccagcagctcgttatcattattattatcacggtatggacgtc

SEQ ID NO: 40 - protein, 17 aa, synthetic construct
  REGION 1..17: note = Synthetic
VRDPAARYHYYYHGMDV

SEQ ID NO: 41 - DNA, 336 bp, synthetic construct
  misc_feature 1..336: note = Synthetic
  source 1..336: mol_type = other DNA
gatattgtgatgactcagtctccactctccctgcccgtcacccctggagagccggcctccatctcctgcaggtctagtcagagcctcctgcacaataatggatataactatttggattggtatctgcagaagccagggcagtctccacagctcctgatctatttgggttctagtcgggcctccggggtccctgacaggttcagtggcagtggatcaggcacagattttatactgaaaatcagcagagtggaggctgaagatgttggggtttattactgcatgcaagctctacaaactccgtggacgttcggccgagggaccaaggtggaaatcaaa

SEQ ID NO: 42 - protein, 112 aa, synthetic construct
  REGION 1..112: note = Synthetic
DIVMTQSPLSLPVTPGEPASISCRSSQSLLHNNGYNYLDWYLQKPGQSPQLLIYLGSSRASGVPDRFSGSGSGTDFILKISRVEAEDVGVYYCMQALQTPWTFGRGTKVEIK

SEQ ID NO: 43 - DNA, 33 bp, synthetic construct
  misc_feature 1..33: note = Synthetic
  source 1..33: mol_type = other DNA
cagagcctcctgcacaataatggatataactat

SEQ ID NO: 44 - protein, 11 aa, synthetic construct
  REGION 1..11: note = Synthetic
QSLLHNNGYNY

SEQ ID NO: 45-46 - skipped (2 entries)

SEQ ID NO: 47 - DNA, 27 bp, synthetic construct
  misc_feature 1..27: note = Synthetic
  source 1..27: mol_type = other DNA
atgcaagctctacaaactccgtggacg

SEQ ID NO: 48 - protein, 9 aa, synthetic construct
  REGION 1..9: note = Synthetic
MQALQTPWT

SEQ ID NO: 49 - DNA, 384 bp, synthetic construct
  misc_feature 1..384: note = Synthetic
  source 1..384: mol_type = other DNA
gaggtgcagctggtggagtctgggggaggcgtggtccagcctgggaggtccctgagactctcctgtgcagcctctggattcaccttcagtagttatgacatgcactgggtccgccaggctccaggcaaggggctggagtgggtggcagttatatcatctgatggacgtgataaatactatgtagactccgtgaagggccgattcaccatctccagagacaactccaagaacacgctttatctgcaaatgaacagcctgagagctgaggacacggctgtttattactgtgcgaaagagatggtgtattacgatattttgactggttatcataactactacggtatggacgtctggggccaagggaccacggtcaccgtctcctca

SEQ ID NO: 50 - protein, 128 aa, synthetic construct
  REGION 1..128: note = Synthetic
EVQLVESGGGVVQPGRSLRLSCAASGFTFSSYDMHWVRQAPGKGLEWVAVISSDGRDKYYVDSVKGRFTISRDNSKNTLYLQMNSLRAEDTAVYYCAKEMVYYDILTGYHNYYGMDVWGQGTTVTVSS

SEQ ID NO: 51 - DNA, 24 bp, synthetic construct
  misc_feature 1..24: note = Synthetic
  source 1..24: mol_type = other DNA
ggattcaccttcagtagttatgac

SEQ ID NO: 52 - protein, 8 aa, synthetic construct
  REGION 1..8: note = Synthetic
GFTFSSYD

SEQ ID NO: 53 - DNA, 24 bp, synthetic construct
  misc_feature 1..24: note = Synthetic
  source 1..24: mol_type = other DNA
atatcatctgatggacgtgataaa

SEQ ID NO: 54 - protein, 8 aa, synthetic construct
  REGION 1..8: note = Synthetic
ISSDGRDK

SEQ ID NO: 55 - DNA, 63 bp, synthetic construct
  misc_feature 1..63: note = Synthetic
  source 1..63: mol_type = other DNA
gcgaaagagatggtgtattacgatattttgactggttatcataactactacggtatggacgtc

SEQ ID NO: 56 - protein, 21 aa, synthetic construct
  REGION 1..21: note = Synthetic
AKEMVYYDILTGYHNYYGMDV

SEQ ID NO: 57 - DNA, 324 bp, synthetic construct
  misc_feature 1..324: note = Synthetic
  source 1..324: mol_type = other DNA
gacatcgtgatgacccagtctccatcctcactgtctgcatctgtaggagacagagtcaccatcacttgtcgggcgagtcagggcattaacaattatttagcctggtttcagcagaaaccagggaaagcccctaagtccctgatccatactgcatccagtttgcaaagtggggtcccatcaaagttcagcggcagtggatctgggacagatttcactctcaccatcagcagcctgcagcctgaagattttgcaacttattactgccaacagtataatacttaccctctcactttcggcggagggaccaaagtggagatcaaacga

SEQ ID NO: 58 - protein, 108 aa, synthetic construct
  REGION 1..108: note = Synthetic
DIVMTQSPSSLSASVGDRVTITCRASQGINNYLAWFQQKPGKAPKSLIHTASSLQSGVPSKFSGSGSGTDFTLTISSLQPEDFATYYCQQYNTYPLTFGGGTKVEIKR

SEQ ID NO: 59 - DNA, 18 bp, synthetic construct
  misc_feature 1..18: note = Synthetic
  source 1..18: mol_type = other DNA
cagggcattaacaattat

SEQ ID NO: 60 - protein, 6 aa, synthetic construct
  REGION 1..6: note = Synthetic
QGINNY

SEQ ID NO: 61-62 - skipped (2 entries)

SEQ ID NO: 63 - DNA, 27 bp, synthetic construct
  misc_feature 1..27: note = Synthetic
  source 1..27: mol_type = other DNA
caacagtataatacttaccctctcact

SEQ ID NO: 64 - protein, 9 aa, synthetic construct
  REGION 1..9: note = Synthetic
QQYNTYPLT

SEQ ID NO: 65 - DNA, 381 bp, synthetic construct
  misc_feature 1..381: note = Synthetic
  source 1..381: mol_type = other DNA
caggtgcagctggtggagtctgggggaggcgtggtccagcctgggaggtccctgagactctcctgtgcagcctctggattcaccttcagtagttatgacatgcactgggtccgccaggctccaggcaaggggctggagtgggtggcagttatatcatctgatggacgtgataaatactatgtagactccgtgaagggccgattcaccatctccagagacaactccaagaacacgctttatctgcaaatgaacagcctgagagctgaggacacggctgtttattactgtgcgaaagagatggtgtattacgatattttgactggttatcataactactacggtatggacgtctggggccaagggaccacggtcaccgtctcc

SEQ ID NO: 66 - protein, 127 aa, synthetic construct
  REGION 1..127: note = Synthetic
QVQLVESGGGVVQPGRSLRLSCAASGFTFSSYDMHWVRQAPGKGLEWVAVISSDGRDKYYVDSVKGRFTISRDNSKNTLYLQMNSLRAEDTAVYYCAKEMVYYDILTGYHNYYGMDVWGQGTTVTVS

SEQ ID NO: 67 - DNA, 321 bp, synthetic construct
  misc_feature 1..321: note = Synthetic
  source 1..321: mol_type = other DNA
gacatccagatgacccagtctccatcctcactgtctgcatctgtaggagacagagtcaccatcacttgtcgggcgagtcagggcattaacaattatttagcctggtttcagcagaaaccagggaaagcccctaagtccctgatccatactgcatccagtttgcaaagtggggtcccatcaaagttcagcggcagtggatctgggacagatttcactctcaccatcagcagcctgcagcctgaagattttgcaacttattactgccaacagtataatacttaccctctcactttcggcggagggaccaaggtggagatcaaa

SEQ ID NO: 68 - protein, 107 aa, synthetic construct
  REGION 1..107: note = Synthetic
DIQMTQSPSSLSASVGDRVTITCRASQGINNYLAWFQQKPGKAPKSLIHTASSLQSGVPSKFSGSGSGTDFTLTISSLQPEDFATYYCQQYNTYPLTFGGGTKVEIK

SEQ ID NO: 69 - DNA, 384 bp, synthetic construct
  misc_feature 1..384: note = Synthetic
  source 1..384: mol_type = other DNA
caggtgcagctggtgcagtctgggggaggcttggtccagcctggggggtccctgagactctcctgtgcagcctccggattcacctttagtaactatttgatgaactgggtccgccaggctccagggaaggggctggagtggctggccaacatacaggaagatggaattgagaaatactatgtggactctgtgaagggccgattcaccatctccagagacaacgccaagaactcactgtatctgcaaatgaacagcctgagagccgaggacacggctgtgtattactgtgcgagagagccctcccattacgatattttgactggttatgactactattacggtatggacgtctggggccaagggaccacggtcaccgtctcctca

SEQ ID NO: 70 - protein, 128 aa, synthetic construct
  REGION 1..128: note = Synthetic
QVQLVQSGGGLVQPGGSLRLSCAASGFTFSNYLMNWVRQAPGKGLEWLANIQEDGIEKYYVDSVKGRFTISRDNAKNSLYLQMNSLRAEDTAVYYCAREPSHYDILTGYDYYYGMDVWGQGTTVTVSS

SEQ ID NO: 71 - DNA, 24 bp, synthetic construct
  misc_feature 1..24: note = Synthetic
  source 1..24: mol_type = other DNA
ggattcacctttagtaactatttg

SEQ ID NO: 72 - protein, 8 aa, synthetic construct
  REGION 1..8: note = Synthetic
GFTFSNYL

SEQ ID NO: 73 - DNA, 24 bp, synthetic construct
  misc_feature 1..24: note = Synthetic
  source 1..24: mol_type = other DNA
atacaggaagatggaattgagaaa

SEQ ID NO: 74 - protein, 8 aa, synthetic construct
  REGION 1..8: note = Synthetic
IQEDGIEK

SEQ ID NO: 75 - DNA, 63 bp, synthetic construct
  misc_feature 1..63: note = Synthetic
  source 1..63: mol_type = other DNA
gcgagagagccctcccattacgatattttgactggttatgactactattacggtatggacgtc

SEQ ID NO: 76 - protein, 21 aa, synthetic construct
  REGION 1..21: note = Synthetic
AREPSHYDILTGYDYYYGMDV

SEQ ID NO: 77 - DNA, 324 bp, synthetic construct
  misc_feature 1..324: note = Synthetic
  source 1..324: mol_type = other DNA
gacatccagttgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcattctcacagtcagcagcctgcagcctgaagactttgcaacttattactgtctacagtataatagtaacccattcactttcggccctgggaccaaggtggagatcaaacga

SEQ ID NO: 78 - protein, 108 aa, synthetic construct
  REGION 1..108: note = Synthetic
DIQLTQSPSSLSASVGDRVTITCRASQGIRNDLGWYQQKPGKAPKRLIYAASSLQSGVPSRFSGSGSGTEFILTVSSLQPEDFATYYCLQYNSNPFTFGPGTKVEIKR

SEQ ID NO: 79 - DNA, 18 bp, synthetic construct
  misc_feature 1..18: note = Synthetic
  source 1..18: mol_type = other DNA
cagggcattagaaatgat

SEQ ID NO: 80 - protein, 6 aa, synthetic construct
  REGION 1..6: note = Synthetic
QGIRND

SEQ ID NO: 81-82 - skipped (2 entries)

SEQ ID NO: 83 - DNA, 27 bp, synthetic construct
  misc_feature 1..27: note = Synthetic
  source 1..27: mol_type = other DNA
ctacagtataatagtaacccattcact

SEQ ID NO: 84 - protein, 9 aa, synthetic construct
  REGION 1..9: note = Synthetic
LQYNSNPFT

SEQ ID NO: 85 - DNA, 381 bp, synthetic construct
  misc_feature 1..381: note = Synthetic
  source 1..381: mol_type = other DNA
gaggtgcagctggtggagtctgggggaggcttggtccagcctggggggtccctgagactctcctgtgcagcctccggattcacctttagtaactatttgatgaactgggtccgccaggctccagggaaggggctggagtggctggccaacatacaggaagatggaattgagaaatactatgtggactctgtgaagggccgattcaccatctccagagacaacgccaagaactcactgtatctgcaaatgaacagcctgagagccgaggacacggctgtgtattactgtgcgagagagccctcccattacgatattttgactggttatgactactattacggtatggacgtctggggccaagggaccacggtcaccgtctcc

SEQ ID NO: 86 - protein, 127 aa, synthetic construct
  REGION 1..127: note = Synthetic
EVQLVESGGGLVQPGGSLRLSCAASGFTFSNYLMNWVRQAPGKGLEWLANIQEDGIEKYYVDSVKGRFTISRDNAKNSLYLQMNSLRAEDTAVYYCAREPSHYDILTGYDYYYGMDVWGQGTTVTVS

SEQ ID NO: 87 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctgatctatgctgcatccagtttgcaaagtggggtcccatcaaggttcagcggcagtggatctgggacagaattcattctcacagtcagcagcctgcagcctgaagactttgcaacttattactgtctacagtataatagtaacccattcactttcggccctgggaccaaagtggatatcaaa

SEQ ID NO: 88 - protein, 107 aa, synthetic construct
  REGION 1..107: note = Synthetic
DIQMTQSPSSLSASVGDRVTITCRASQGIRNDLGWYQQKPGKAPKRLIYAASSLQSGVPSRFSGSGSGTEFILTVSSLQPEDFATYYCLQYNSNPFTFGPGTKVDIK

SEQ ID NO: 89 - DNA, 381 bp, synthetic construct
  misc_feature 1..381: note = Synthetic
  source 1..381: mol_type = other DNA
gaggtgcagctggtgcagtctgggggagccttggtacagcctggggggtccctgagactctcctgtacagcctctggtttcaccttcagtaactacgacatgcactgggtccgccaaactacaggaaaaggtctggagtggatctcagctattgatactgctggtgacacatactatccaggctccgtgaagggccgattcaccgtctccagagaaaatgccaagaactccttttatcttcaaatgaacagcctgagagccggggacacggctgtgtattactgtgcaagggaggggaagtattacgatattttgactggtgactaccactactacggtatggacgtctggggccaagggaccacggtcaccgtctcctca

SEQ ID NO: 90 - protein, 127 aa, synthetic construct
  REGION 1..127: note = Synthetic
EVQLVQSGGALVQPGGSLRLSCTASGFTFSNYDMHWVRQTTGKGLEWISAIDTAGDTYYPGSVKGRFTVSRENAKNSFYLQMNSLRAGDTAVYYCAREGKYYDILTGDYHYYGMDVWGQGTTVTVSS

SEQ ID NO: 91 - DNA, 24 bp, synthetic construct
  misc_feature 1..24: note = Synthetic
  source 1..24: mol_type = other DNA
ggtttcaccttcagtaactacgac

SEQ ID NO: 92 - protein, 8 aa, synthetic construct
  REGION 1..8: note = Synthetic
GFTFSNYD

SEQ ID NO: 93 - DNA, 21 bp, synthetic construct
  misc_feature 1..21: note = Synthetic
  source 1..21: mol_type = other DNA
attgatactgctggtgacaca

SEQ ID NO: 94 - protein, 7 aa, synthetic construct
  REGION 1..7: note = Synthetic
IDTAGDT

SEQ ID NO: 95 - DNA, 63 bp, synthetic construct
  misc_feature 1..63: note = Synthetic
  source 1..63: mol_type = other DNA
gcaagggaggggaagtattacgatattttgactggtgactaccactactacggtatggacgtc

SEQ ID NO: 96 - protein, 21 aa, synthetic construct
  REGION 1..21: note = Synthetic
AREGKYYDILTGDYHYYGMDV

SEQ ID NO: 97 - DNA, 324 bp, synthetic construct
  misc_feature 1..324: note = Synthetic
  source 1..324: mol_type = other DNA
gccatccggatgacccagtctccatcctccctgtctgcatctgtaggagacagagtcaccatcacttgtcgggcaagtcagggcattagaaatgatttaggctggtatcagcagaaaccagggaaagcccctaagcgactgatctatgctacatccagtttgcaaagtggggtcccatcaaggttcagcggcagtggatctgggacagaattcactctcacaatcagcagcctgcagcctgaagattttgcaacttattactgtctacagcataatagttacccgctcactttcggcggagggaccaaggtggaaatcaaacga

SEQ ID NO: 98 - protein, 108 aa, synthetic construct
  REGION 1..108: note = Synthetic
AIRMTQSPSSLSASVGDRVTITCRASQGIRNDLGWYQQKPGKAPKRLIYATSSLQSGVPSRFSGSGSGTEFTLTISSLQPEDFATYYCLQHNSYPLTFGGGTKVEIKR

SEQ ID NO: 99 - DNA, 18 bp, synthetic construct
  misc_feature 1..18: note = Synthetic
  source 1..18: mol_type = other DNA
cagggcattagaaatgat

SEQ ID NO: 100 - protein, 6 aa, synthetic construct
  REGION 1..6: note = Synthetic
QGIRND

SEQ ID NO: 101-102 - skipped (2 entries)

SEQ ID NO: 103 - DNA, 27 bp, synthetic construct
  misc_feature 1..27: note = Synthetic
  source 1..27: mol_type = other DNA
ctacagcataatagttacccgctcact

SEQ ID NO: 104 - protein, 9 aa, synthetic construct
  REGION 1..9: note = Synthetic
LQHNSYPLT

SEQ ID NO: 105 - DNA, 378 bp, synthetic construct
  misc_feature 1..378: note = Synthetic
  source 1..378: mol_type = other DNA
gaggtgcagctggtggagtctgggggagccttggtacagcctggggggtccctgagactctcctgtacagcctctggtttcaccttcagtaactacgacatgcactgggtccgccaaactacaggaaaaggtctggagtggatctcagctattgatactgctggtgacacatactatccaggctccgtgaagggccgattcaccgtctccagagaaaatgccaagaactccttttatcttcaaatgaacagcctgagagccggggacacggctgtgtattactgtgcaagggaggggaagtattacgatattttgactggtgactaccactactacggtatggacgtctggggccaagggaccacggtcaccgtctcc

SEQ ID NO: 106 - protein, 126 aa, synthetic construct
  REGION 1..126: note = Synthetic
EVQLVESGGALVQPGGSLRLSCTASGFTFSNYDMHWVRQTTGKGLEWISAIDTAGDTYYPGSVKGRFTVSRENAKNSFYLQMNSLRAGDTAVYYCAREGKYYDILTGDYHYYGMDVWGQGTTVTVS

SEQ ID NO: 107 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgtcgggcaagtcagggcattagaaatgatttaggctggtatcagcagaaaccagggaaagcccctaagcgactgatctatgctacatccagtttgcaaagtggggtcccatcaaggttcagcggcagtggatctgggacagaattcactctcacaatcagcagcctgcagcctgaagattttgcaacttattactgtctacagcataatagttacccgctcactttcggcggagggaccaagctggagatcaaa

SEQ ID NO: 108 - protein, 107 aa, synthetic construct
  REGION 1..107: note = Synthetic
DIQMTQSPSSLSASVGDRVTITCRASQGIRNDLGWYQQKPGKAPKRLIYATSSLQSGVPSRFSGSGSGTEFTLTISSLQPEDFATYYCLQHNSYPLTFGGGTKLEIK

SEQ ID NO: 109 - DNA, 372 bp, synthetic construct
  misc_feature 1..372: note = Synthetic
  source 1..372: mol_type = other DNA
caggtgcagctggtgcagtctgggggaggcgtggtccagcctgggaggtccctgagactctcctgtgcagcgtctgggttcacctttagtaactttggcatgcactgggtccgccaggctccaggcaaggggctggagtgggtggcagttatatggtttgatgaaattgataaatactatgcagactccgtgaagggccgattcaccatctccagagacaattccaagaacacgctgtatccgcaaatgaacagcctgagagccgaagacacggctgtgtattactgtgcgcgagaagattacgatattttgactggttactattacgctatggacgtctggggccaagggaccacggtcaccgtctcctca

SEQ ID NO: 110 - protein, 124 aa, synthetic construct
  REGION 1..124: note = Synthetic
QVQLVQSGGGVVQPGRSLRLSCAASGFTFSNFGMHWVRQAPGKGLEWVAVIWFDEIDKYYADSVKGRFTISRDNSKNTLYPQMNSLRAEDTAVYYCAREDYDILTGYYYAMDVWGQGTTVTVSS

SEQ ID NO: 111 - DNA, 24 bp, synthetic construct
  misc_feature 1..24: note = Synthetic
  source 1..24: mol_type = other DNA
gggttcacctttagtaactttggc

SEQ ID NO: 112 - protein, 8 aa, synthetic construct
  REGION 1..8: note = Synthetic
GFTFSNFG

SEQ ID NO: 113 - DNA, 24 bp, synthetic construct
  misc_feature 1..24: note = Synthetic
  source 1..24: mol_type = other DNA
atatggtttgatgaaattgataaa

SEQ ID NO: 114 - protein, 8 aa, synthetic construct
  REGION 1..8: note = Synthetic
IWFDEIDK

SEQ ID NO: 115 - DNA, 51 bp, synthetic construct
  misc_feature 1..51: note = Synthetic
  source 1..51: mol_type = other DNA
gcgcgagaagattacgatattttgactggttactattacgctatggacgtc

SEQ ID NO: 116 - protein, 17 aa, synthetic construct
  REGION 1..17: note = Synthetic
AREDYDILTGYYYAMDV

SEQ ID NO: 117 - DNA, 321 bp, synthetic construct
  misc_feature 1..321: note = Synthetic
  source 1..321: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctaatctatgctgcatcccgtttgcaaagtggggtcccatcgaggttcagcggcagtggatctgggacagaattcactctcacaatcagcagcctgcagcctgaagattttggaacttattactgtctacagcataatagtcaccccaccttcggccaagggaccaaggtggagatcaaacga

SEQ ID NO: 118 - protein, 107 aa, synthetic construct
  REGION 1..107: note = Synthetic
DIQMTQSPSSLSASVGDRVTITCRASQGIRNDLGWYQQKPGKAPKRLIYAASRLQSGVPSRFSGSGSGTEFTLTISSLQPEDFGTYYCLQHNSHPTFGQGTKVEIKR

SEQ ID NO: 119 - DNA, 18 bp, synthetic construct
  misc_feature 1..18: note = Synthetic
  source 1..18: mol_type = other DNA
cagggcattagaaatgat

SEQ ID NO: 120 - protein, 6 aa, synthetic construct
  REGION 1..6: note = Synthetic
QGIRND

SEQ ID NO: 121-122 - skipped (2 entries)

SEQ ID NO: 123 - DNA, 24 bp, synthetic construct
  misc_feature 1..24: note = Synthetic
  source 1..24: mol_type = other DNA
ctacagcataatagtcaccccacc

SEQ ID NO: 124 - protein, 8 aa, synthetic construct
  REGION 1..8: note = Synthetic
LQHNSHPT

SEQ ID NO: 125 - DNA, 369 bp, synthetic construct
  misc_feature 1..369: note = Synthetic
  source 1..369: mol_type = other DNA
caggtgcagctggtggagtctgggggaggcgtggtccagcctgggaggtccctgagactctcctgtgcagcgtctgggttcacctttagtaactttggcatgcactgggtccgccaggctccaggcaaggggctggagtgggtggcagttatatggtttgatgaaattgataaatactatgcagactccgtgaagggccgattcaccatctccagagacaattccaagaacacgctgtatccgcaaatgaacagcctgagagccgaagacacggctgtgtattactgtgcgcgagaagattacgatattttgactggttactattacgctatggacgtctggggccaagggaccacggtcaccgtctcc

SEQ ID NO: 126 - protein, 123 aa, synthetic construct
  REGION 1..123: note = Synthetic
QVQLVESGGGVVQPGRSLRLSCAASGFTFSNFGMHWVRQAPGKGLEWVAVIWFDEIDKYYADSVKGRFTISRDNSKNTLYPQMNSLRAEDTAVYYCAREDYDILTGYYYAMDVWGQGTTVTVS

SEQ ID NO: 127 - DNA, 318 bp, synthetic construct
  misc_feature 1..318: note = Synthetic
  source 1..318: mol_type = other DNA
gacatccagatgacccagtctccatcctccctgtctgcatctgtaggagacagagtcaccatcacttgccgggcaagtcagggcattagaaatgatttaggctggtatcagcagaaaccagggaaagcccctaagcgcctaatctatgctgcatcccgtttgcaaagtggggtcccatcgaggttcagcggcagtggatctgggacagaattcactctcacaatcagcagcctgcagcctgaagattttggaacttattactgtctacagcataatagtcaccccaccttcggccaagggaccaaggtggagatcaaa

SEQ ID NO: 128 - protein, 106 aa, synthetic construct
  REGION 1..106: note = Synthetic
DIQMTQSPSSLSASVGDRVTITCRASQGIKNDLGWYQQKPGKAPKRLIYAASRLQSGVPSRFSGSGSGTEFTLTISSLQPEDFGTYYCLQHNSHPTFGQGTKVEIK

SEQ ID NO: 129 - DNA, 381 bp, synthetic construct
  misc_feature 1..381: note = Synthetic
  source 1..381: mol_type = other DNA
gaggtgcagctggtggagtcggggggaggcatggtacagcctggggggtccctgagactctcctgtgcagcctctggattcacctccagtaactacgacatgcactgggtccgccaagctacaggaaaaggtctggagtgggtctcaagtattgatactgctggggacacttactatccagactccgtgaagggccgctttatcatctccagagaaaatgccaaaaactccctgtatcttcaaatgaatagcctgagagccggggacacggctgtgtattactgtacaagggagccccgaaattacgaaattttgactggtcactaccactaccacggtatggacatctggggccaagggaccacggtcaccgtctcctca

SEQ ID NO: 130 - protein, 127 aa, synthetic construct
  REGION 1..127: note = Synthetic
EVQLVESGGGMVQPGGSLRLSCAASGFTSSNYDMHWVRQATGKGLEWVSSIDTAGDTYYPDSVKGRFIISRENAKNSLYLQMNSLRAGDTAVYYCTREPRNYEILTGHYHYHGMDIWGQGTTVTVSS

SEQ ID NO: 131 - DNA, 24 bp, synthetic construct
  misc_feature 1..24: note = Synthetic
  source 1..24: mol_type = other DNA
ggattcacctccagtaactacgac

SEQ ID NO: 132 - protein, 8 aa, synthetic construct
  REGION 1..8: note = Synthetic
GFTSSNYD

SEQ ID NO: 133 - DNA, 21 bp, synthetic construct
  misc_feature 1..21: note = Synthetic
  source 1..21: mol_type = other DNA
attgatactgctggggacact

SEQ ID NO: 134 - protein, 7 aa, synthetic construct
  REGION 1..7: note = Synthetic
IDTAGDT

SEQ ID NO: 135 - DNA, 63 bp, synthetic construct
  misc_feature 1..63: note = Synthetic
  source 1..63: mol_type = other DNA
acaagggagccccgaaattacgaaattttgactggtcactaccactaccacggtatggacatc

SEQ ID NO: 136 - protein, 21 aa, synthetic construct
  REGION 1..21: note = Synthetic
TREPRNYEILTGHYHYHGMDI

SEQ ID NO: 137 - DNA, 324 bp, synthetic construct
  misc_feature 1..324: note = Synthetic
  source 1..324: mol_type = other DNA
gacatccagatgacccagtcgccatcctccctgtctgcatctgtaggagacagagtcaccatcacttgccgggcaagtcaggccattagaaatgatttaggctggtatcagcagaaaccagggaaagcccctaaactcctgatctatactgcattcagtttacagagtggggtcccatcaaggttcagcggcagtaaatctggcacagacttcactctcaccatcagcagcctgcagcctgaagattttgcgacttattactgtctgcaggattacactaatcctcggacgttcggccaagggaccaaggtggagatcaaacga

SEQ ID NO: 138 - protein, 108 aa, synthetic construct
  REGION 1..108: note = Synthetic
DIQMTQSPSSLSASVGDRVTITCRASQAIRNDLGWYQQKPGKAPKLLIYTAFSLQSGVPSRFSGSKSGTDFTLTISSLQPEDFATYYCLQDYTNPRTFGQGTKVEIKR

SEQ ID NO: 139 - DNA, 18 bp, synthetic construct
  misc_feature 1..18: note = Synthetic
  source 1..18: mol_type = other DNA
caggccattagaaatgat

SEQ ID NO: 140 - protein, 6 aa, synthetic construct
  REGION 1..6: note = Synthetic
QAIRND

SEQ ID NO: 141-142 - skipped (2 entries)

SEQ ID NO: 143 - DNA, 27 bp, synthetic construct
  misc_feature 1..27: note = Synthetic
  source 1..27: mol_type = other DNA
ctgcaggattacactaatcctcggacg

SEQ ID NO: 144 - protein, 9 aa, synthetic construct
  REGION 1..9: note = Synthetic
LQDYTNPRT

SEQ ID NO: 145 - DNA, 378 bp, synthetic construct
  misc_feature 1..378: note = Synthetic
  source 1..378: mol_type = other DNA
gaggtgcagctggtggagtcggggggaggcatggtacagcctggggggtccctgagactctcctgtgcagcctctggattcacctccagtaactacgacatgcactgggtccgccaagctacaggaaaaggtctggagtgggtctcaagtattgatactgctggggacacttactatccagactccgtgaagggccgctttatcatctccagagaaaatgccaaaaactccctgtatcttcaaatgaatagcctgagagccggggacacggctgtgtattactgtacaagggagccccgaaattacgaaattttgactggtcactaccactaccacggtatggacatctggggccaagggaccacggtcaccgtctcc

SEQ ID NO: 146 - protein, 126 aa, synthetic construct
  REGION 1..126: note = Synthetic
EVQLVESGGGMVQPGGSLRLSCAASGFTSSNYDMHWVRQATGKGLEWVSSIDTAGDTYYPDSVKGRFIISRENAKNSLYLQMNSLRAGDTAVYYCTREPRNYEILTGHYHYHGMDIWGQGTTVTVS

SEQ ID NO: 147 - DNA, 321 bp, synthetic construct
  misc_feature 1..321: note = Synthetic
  source 1..321: mol_type = other DNA
gccatccagatgacccagtcgccatcctccctgtctgcatctgtaggagacagagtcaccatcacttgccgggcaagtcaggccattagaaatgatttaggctggtatcagcagaaaccagggaaagcccctaaactcctgatctatactgcattcagtttacagagtggggtcccatcaaggttcagcggcagtaaatctggcacagacttcactctcaccatcagcagcctgcagcctgaagattttgcgacttattactgtctgcaggattacactaatcctcggacgttcggccaagggaccaaggtggaaatcaaa

SEQ ID NO: 148 - protein, 107 aa, synthetic construct
  REGION 1..107: note = Synthetic
AIQMTQSPSSLSASVGDRVTITCRASQAIRNDLGWYQQKPGKAPKLLIYTAFSLQSGVPSRFSGSKSGTDFTLTISSLQPEDFATYYCLQDYTNPRTFGQGTKVEIK

SEQ ID NO: 149 - DNA, 357 bp, synthetic construct
  misc_feature 1..357: note = synthetic
  source 1..357: mol_type = other DNA
caggtgcagctggtggagtctgggggaggcgtggtccagcctgggaggtccctgagactctcctgtgcagcctctggattcgccttcagtaactatggcatgcactgggtccgccaggctccaggcaaggggctggaatgggtgacatttatatcatatgatggaagtaataaatactatgcagactccgtgaagggccgattcaccatctccagagacaattccaagaacacgctgtatctgcaagtgaacagcctgagagctgaggacacggctgtgtattactgtgcgaaagaagcagtattagctgccctctttgactactggggccagggaaccctggtcaccgtctcctca

SEQ ID NO: 150 - protein, 119 aa, synthetic construct
  REGION 1..119: note = synthetic
QVQLVESGGGVVQPGRSLRLSCAASGFAFSNYGMHWVRQAPGKGLEWVTFISYDGSNKYYADSVKGRFTISRDNSKNTLYLQVNSLRAEDTAVYYCAKEAVLAALFDYWGQGTLVTVSS

SEQ ID NO: 151 - DNA, 24 bp, synthetic construct
  misc_feature 1..24: note = synthetic
  source 1..24: mol_type = other DNA
ggattcgccttcagtaactatggc

SEQ ID NO: 152 - protein, 8 aa, synthetic construct
  REGION 1..8: note = synthetic
GFAFSNYG

SEQ ID NO: 153 - DNA, 24 bp, synthetic construct
  misc_feature 1..24: note = synthetic
  source 1..24: mol_type = other DNA
atatcatatgatggaagtaataaa

SEQ ID NO: 154 - protein, 8 aa, synthetic construct
  REGION 1..8: note = synthetic
ISYDGSNK

SEQ ID NO: 155 - DNA, 36 bp, synthetic construct
  misc_feature 1..36: note = synthetic
  source 1..36: mol_type = other DNA
gcgaaagaagcagtattagctgccctctttgactac

SEQ ID NO: 156 - protein, 12 aa, synthetic construct
  REGION 1..12: note = synthetic
AKEAVLAALFDY

SEQ ID NO: 157 - DNA, 336 bp, synthetic construct
  misc_feature 1..336: note = synthetic
  source 1..336: mol_type = other DNA
gacatcgtgatgacccagtctccagactccctggctgtgtctctgggcgagagggccaccatcaactgcaagtccagccagagtgttttatacagctccaacaatcagaactacttagcttggtaccagcagaaaccaggacagcctcctaagctgctcatttactgggcatctacccgggaatccggggtccctgaccgattcagtggcagcgggtctgggacagatttcactctcaccatcaacagcctgcaggctgaagatgtggcagtttattactgtcagcaatattatagtactcctacgttcggccaagggaccaaggtggaaatcaaa

SEQ ID NO: 158 - protein, 112 aa, synthetic construct
  REGION 1..112: note = synthetic
DIVMTQSPDSLAVSLGERATINCKSSQSVLYSSNNQNYLAWYQQKPGQPPKLLIYWASTRESGVPDRFSGSGSGTDFTLTINSLQAEDVAVYYCQQYYSTPTFGQGTKVEIK

SEQ ID NO: 159 - DNA, 36 bp, synthetic construct
  misc_feature 1..36: note = synthetic
  source 1..36: mol_type = other DNA
cagagtgttttatacagctccaacaatcagaactac

SEQ ID NO: 160 - protein, 12 aa, synthetic construct
  REGION 1..12: note = synthetic
QSVLYSSNNQNY

SEQ ID NO: 161-162 - skipped (2 entries)

SEQ ID NO: 163 - DNA, 24 bp, synthetic construct
  misc_feature 1..24: note = synthetic
  source 1..24: mol_type = other DNA
cagcaatattatagtactcctacg

SEQ ID NO: 164 - protein, 8 aa, synthetic construct
  REGION 1..8: note = synthetic
QQYYSTPT

SEQ ID NO: 165 - DNA, 357 bp, synthetic construct
  misc_feature 1..357: note = synthetic
  source 1..357: mol_type = other DNA
caggtgcagctggtggagtctgggggaggcgtggtccagcctgggaggtccctgagactctcctgtgcagcctctggattcacgttcaatacctatggcatgcactgggtccgccaggctccagtcaaggggctggagtgggtggcatttatatcaaatgataagagtaatacattctatgcagactccgtgaagggccgattcaccatctccagagacaattccaagaacacgctgtttctggaaatgaacagcctgacagctgaggacacggctgtttattactgtgcgaaagagtccattttagcagccctctttgactactggggccagggaaccctggtcactgtctcctca

SEQ ID NO: 166 - protein, 119 aa, synthetic construct
  REGION 1..119: note = synthetic
QVQLVESGGGVVQPGRSLRLSCAASGFTFNTYGMHWVRQAPVKGLEWVAFISNDKSNTFYADSVKGRFTISRDNSKNTLFLEMNSLTAEDTAVYYCAKESILAALFDYWGQGTLVTVSS

SEQ ID NO: 167 - DNA, 24 bp, synthetic construct
  misc_feature 1..24: note = synthetic
  source 1..24: mol_type = other DNA
ggattcacgttcaatacctatggc

SEQ ID NO: 168 - protein, 8 aa, synthetic construct
  REGION 1..8: note = synthetic
GFTFNTYG

SEQ ID NO: 169 - DNA, 24 bp, synthetic construct
  misc_feature 1..24: note = synthetic
  source 1..24: mol_type = other DNA
atatcaaatgataagagtaataca

SEQ ID NO: 170 - protein, 8 aa, synthetic construct
  REGION 1..8: note = synthetic
ISNDKSNT

SEQ ID NO: 171 - DNA, 36 bp, synthetic construct
  misc_feature 1..36: note = synthetic
  source 1..36: mol_type = other DNA
gcgaaagagtccattttagcagccctctttgactac

SEQ ID NO: 172 - protein, 12 aa, synthetic construct
  REGION 1..12: note = synthetic
AKESILAALFDY

SEQ ID NO: 173 - DNA, 336 bp, synthetic construct
  misc_feature 1..336: note = synthetic
  source 1..336: mol_type = other DNA
gacatcgtgatgacccagtctccagactccctggctgtgtctctgggcgagagggccaccatcaactgcaagtccagccagagtgttttatacagctccaacaataagaattacttagcttggtaccaacagaaaccaagacagcctcttaaactactcatttactgggcatctattcgggaatccggggtccctgaccgattcagtggcagcgggtctgggacagatttcactctcaccatcagcagcctgcaggctgaagatgtggcagtttattactgtcagcaattttatagtgttcccacttttggccaggggaccaagctggagatcaaa

SEQ ID NO: 174 - protein, 112 aa, synthetic construct
  REGION 1..112: note = synthetic
DIVMTQSPDSLAVSLGERATINCKSSQSVLYSSNNKNYLAWYQQKPRQPLKLLIYWASIRESGVPDRFSGSGSGTDFTLTISSLQAEDVAVYYCQQFYSVPTFGQGTKLEIK

SEQ ID NO: 175 - DNA, 36 bp, synthetic construct
  misc_feature 1..36: note = synthetic
  source 1..36: mol_type = other DNA
cagagtgttttatacagctccaacaataagaattac

SEQ ID NO: 176 - protein, 12 aa, synthetic construct
  REGION 1..12: note = synthetic
QSVLYSSNNKNY

SEQ ID NO: 177-178 - skipped (2 entries)

SEQ ID NO: 179 - DNA, 24 bp, synthetic construct
  misc_feature 1..24: note = synthetic
  source 1..24: mol_type = other DNA
cagcaattttatagtgttcccact

SEQ ID NO: 180 - protein, 8 aa, synthetic construct
  REGION 1..8: note = synthetic
QQFYSVPT

SEQ ID NO: 181 - DNA, 357 bp, synthetic construct
  misc_feature 1..357: note = synthetic
  source 1..357: mol_type = other DNA
caggtgcagctggtggagtctgggggaggcgtggtccagcctgggaggtccctgagactctcctgtgcagcctctggattcacgtttagtacctttggcatgcactgggtccgccaggctccagtcaaggggctggagtgggtggcttttatatcaaatgataagaataataaattctatgcagactccgtgaagggccgattcaccatctccagagacaattccagggacacgctatatctgcaaatgaacagcctgacacctgaggacacggctgtttattactgtgcgaaagagtccattttagcagccctctttgactactggggccagggaaccctggtcaccgtctcctca

SEQ ID NO: 182 - protein, 119 aa, synthetic construct
  REGION 1..119: note = synthetic
QVQLVESGGGVVQPGRSLRLSCAASGFTFSTFGMHWVRQAPVKGLEWVAFISNDKNNKFYADSVKGRFTISRDNSRDTLYLQMNSLTPEDTAVYYCAKESILAALFDYWGQGTLVTVSS

SEQ ID NO: 183 - DNA, 24 bp, synthetic construct
  misc_feature 1..24: note = synthetic
  source 1..24: mol_type = other DNA
ggattcacgtttagtacctttggc

SEQ ID NO: 184 - protein, 8 aa, synthetic construct
  REGION 1..8: note = synthetic
GFTFSTFG

SEQ ID NO: 185 - DNA, 24 bp, synthetic construct
  misc_feature 1..24: note = synthetic
  source 1..24: mol_type = other DNA
atatcaaatgataagaataataaa

SEQ ID NO: 186 - protein, 8 aa, synthetic construct
  REGION 1..8: note = synthetic
ISNDKNNK

SEQ ID NO: 187 - DNA, 36 bp, synthetic construct; identical to SEQ ID NO: 171
  misc_feature 1..36: note = synthetic
  source 1..36: mol_type = other DNA

SEQ ID NO: 188 - protein, 12 aa, synthetic construct
  REGION 1..12: note = synthetic
AKESILAALFDY

SEQ ID NO: 189 - DNA, 336 bp, synthetic construct
  misc_feature 1..336: note = synthetic
  source 1..336: mol_type = other DNA
gacatcgtgatgacccagtctccagactccctggctgtgtctctgggcgagagggccaccatcaactgcaagtccagccagagtgttttatacagctccaacaataaaaattacttagcttggtaccagcagaaaccaggacagcctcttaaacttctcatttactgggcatctattcgggaatccggggtccctgaccgattcagtggcagcgggtctgggacagatttcactctcaccatcagcagcctgcaggctgaagatgtggcagtttattactgtcagcaattttatactgttcccacttttggcctggggaccaagctggagatcaaa

SEQ ID NO: 190 - protein, 112 aa, synthetic construct
  REGION 1..112: note = synthetic
DIVMTQSPDSLAVSLGERATINCKSSQSVLYSSNNKNYLAWYQQKPGQPLKLLIYWASIRESGVPDRFSGSGSGTDFTLTISSLQAEDVAVYYCQQFYTVPTFGLGTKLEIK

SEQ ID NO: 191 - DNA, 36 bp, synthetic construct
  misc_feature 1..36: note = synthetic
  source 1..36: mol_type = other DNA
cagagtgttttatacagctccaacaataaaaattac

SEQ ID NO: 192 - protein, 12 aa, synthetic construct
  REGION 1..12: note = synthetic
QSVLYSSNNKNY

SEQ ID NO: 193-194 - skipped (2 entries)

SEQ ID NO: 195 - DNA, 24 bp, synthetic construct
  misc_feature 1..24: note = synthetic
  source 1..24: mol_type = other DNA
cagcaattttatactgttcccact

SEQ ID NO: 196 - protein, 8 aa, synthetic construct
  REGION 1..8: note = synthetic
QQFYTVPT

SEQ ID NO: 197 - DNA, 357 bp, synthetic construct
  misc_feature 1..357: note = synthetic
  source 1..357: mol_type = other DNA
caggtgcagctggtggagtctgggggaggcgtggtccagcctgggaggtccctgagactctcctgtgtagcctctggattcaccttcaggaactatgacatgcactgggtccgccaggctcctggcaaggggctggaatgggtggcagttacatcatctgatggacttaataaattctattcagactccgtgaagggccgattcaccatctccagagacaattccaagaacacgctgtctctgcaaattaccggcctgagagctgaggacacggctgtgtattactgtgcgaaagagtccattttagcagccctctttgactactggggccagggaaccctggtcaccgtctcctca

SEQ ID NO: 198 - protein, 119 aa, synthetic construct
  REGION 1..119: note = synthetic
QVQLVESGGGVVQPGRSLRLSCVASGFTFRNYDMHWVRQAPGKGLEWVAVTSSDGLNKFYSDSVKGRFTISRDNSKNTLSLQITGLRAEDTAVYYCAKESILAALFDYWGQGTLVTVSS

SEQ ID NO: 199 - DNA, 24 bp, synthetic construct
  misc_feature 1..24: note = synthetic
  source 1..24: mol_type = other DNA
ggattcaccttcaggaactatgac

SEQ ID NO: 200 - protein, 8 aa, synthetic construct
  REGION 1..8: note = synthetic
GFTFRNYD

SEQ ID NO: 201 - DNA, 24 bp, synthetic construct
  misc_feature 1..24: note = synthetic
  source 1..24: mol_type = other DNA
acatcatctgatggacttaataaa

SEQ ID NO: 202 - protein, 8 aa, synthetic construct
  REGION 1..8: note = synthetic
TSSDGLNK

SEQ ID NO: 203 - DNA, 36 bp, synthetic construct; identical to SEQ ID NO: 171
  misc_feature 1..36: note = synthetic
  source 1..36: mol_type = other DNA

SEQ ID NO: 204 - protein, 12 aa, synthetic construct
  REGION 1..12: note = synthetic
AKESILAALFDY

SEQ ID NO: 205 - DNA, 336 bp, synthetic construct
  misc_feature 1..336: note = synthetic
  source 1..336: mol_type = other DNA
gacatcgtgatgacccagtctccagactccctggctgtgtctctgggcgagagggccaccatcaactgcaagtccagccagagtgttttatacagctccaacaataagaactacttggcttggtaccagcagaaaccaggacagcctcctaagctgctcttttactgggcatctacccgggaatccggggtccctgaccgattcagtggcagcgggtctgggacagatttcactctcaccatcagcagcctgcaggctgaagatgtggcagtttattactgtcagcaacattatactactcccacttttggccaggggaccaagctggagatcaaa

SEQ ID NO: 206 - protein, 112 aa, synthetic construct
  REGION 1..112: note = synthetic
DIVMTQSPDSLAVSLGERATINCKSSQSVLYSSNNKNYLAWYQQKPGQPPKLLFYWASTRESGVPDRFSGSGSGTDFTLTISSLQAEDVAVYYCQQHYTTPTFGQGTKLEIK

SEQ ID NO: 207 - DNA, 36 bp, synthetic construct
  misc_feature 1..36: note = synthetic
  source 1..36: mol_type = other DNA
cagagtgttttatacagctccaacaataagaactac

SEQ ID NO: 208 - protein, 12 aa, synthetic construct
  REGION 1..12: note = synthetic
QSVLYSSNNKNY

SEQ ID NO: 209-210 - skipped (2 entries)

SEQ ID NO: 211 - DNA, 24 bp, synthetic construct
  misc_feature 1..24: note = synthetic
  source 1..24: mol_type = other DNA
cagcaacattatactactcccact

SEQ ID NO: 212 - protein, 8 aa, synthetic construct
  REGION 1..8: note = synthetic
QQHYTTPT

SEQ ID NO: 213 - DNA, 357 bp, synthetic construct
  misc_feature 1..357: note = synthetic
  source 1..357: mol_type = other DNA
caggtgcagctggtggagtctgggggaggcgtggtccagcctgggaggtccctgagactctcctgtgcagcctctggattcaccttcagtagctatggcatgcactgggtccgccagactccgggcaaggggctggagtgggtggcatttatatcatatgatggaaataataaatactatgcagactccgtgaagggccgattcaccatctccagagacaattccaagaacacgctgtttctgcaaatgaacagcctgagagctgaggacacggctgtgtattactgtgcgaaagagtccattttagcagccctctttgactactggggccagggaaccctggtcaccgtctcctca

SEQ ID NO: 214 - protein, 119 aa, synthetic construct
  REGION 1..119: note = synthetic
QVQLVESGGGVVQPGRSLRLSCAASGFTFSSYGMHWVRQTPGKGLEWVAFISYDGNNKYYADSVKGRFTISRDNSKNTLFLQMNSLRAEDTAVYYCAKESILAALFDYWGQGTLVTVSS

SEQ ID NO: 215 - DNA, 24 bp, synthetic construct
  misc_feature 1..24: note = synthetic
  source 1..24: mol_type = other DNA
ggattcaccttcagtagctatggc

SEQ ID NO: 216 - protein, 8 aa, synthetic construct
  REGION 1..8: note = synthetic
GFTFSSYG

SEQ ID NO: 217 - DNA, 24 bp, synthetic construct
  misc_feature 1..24: note = synthetic
  source 1..24: mol_type = other DNA
atatcatatgatggaaataataaa

SEQ ID NO: 218 - protein, 8 aa, synthetic construct
  REGION 1..8: note = synthetic
ISYDGNNK

SEQ ID NO: 219 - DNA, 36 bp, synthetic construct; identical to SEQ ID NO: 171
  misc_feature 1..36: note = synthetic
  source 1..36: mol_type = other DNA

SEQ ID NO: 220 - protein, 12 aa, synthetic construct
  REGION 1..12: note = synthetic
AKESILAALFDY

SEQ ID NO: 221 - DNA, 336 bp, synthetic construct
  misc_feature 1..336: note = synthetic
  source 1..336: mol_type = other DNA
gacatcgtgatgacccagtctccagactccctggctgtgtctctgggcgagagggccaccatcaactgcaagtccagccagagtgttttatacagctccaacaataagaactacttagcttggtaccagcagaaacctggacagcctcctaagctgctcatttactgggcatctacccgggaatccggggtccctgaccgattcagtggcagcgggtctgggacagatttcactctcaccatcagcagcctgcaggctgaagatgtggcactttattactgtcaacaatattataatactcccacttttggccaggggaccaagctggagatcaaa

SEQ ID NO: 222 - protein, 112 aa, synthetic construct
  REGION 1..112: note = synthetic
DIVMTQSPDSLAVSLGERATINCKSSQSVLYSSNNKNYLAWYQQKPGQPPKLLIYWASTRESGVPDRFSGSGSGTDFTLTISSLQAEDVALYYCQQYYNTPTFGQGTKLEIK

SEQ ID NO: 223 - DNA, 36 bp, synthetic construct; identical to SEQ ID NO: 207
  misc_feature 1..36: note = synthetic
  source 1..36: mol_type = other DNA

SEQ ID NO: 224 - protein, 12 aa, synthetic construct
  REGION 1..12: note = synthetic
QSVLYSSNNKNY

SEQ ID NO: 225-226 - skipped (2 entries)

SEQ ID NO: 227 - DNA, 24 bp, synthetic construct
  misc_feature 1..24: note = synthetic
  source 1..24: mol_type = other DNA
caacaatattataatactcccact

SEQ ID NO: 228 - protein, 8 aa, synthetic construct
  REGION 1..8: note = synthetic
QQYYNTPT

SEQ ID NO: 229 - DNA, 357 bp, synthetic construct
  misc_feature 1..357: note = synthetic
  source 1..357: mol_type = other DNA
caggtgcagctggtggagtctgggggaggcgtggtccagcctgggaggtccctgagactctcctgtgcagcctctggattcaccttcagtagctatggcatgcactgggtccgccaggctccagtcaaggggctggagtgggtggcatttatatcatatgatggaagtaataaatactatgcagactccgtgaagggccgattcaccatctccagagacaattccaagaacacgctgtatctccaaatgaacagcctgacagctgaggacacggctgtttattactgtgcgaaagagtccattttagcagccctctttgactactggggccagggaaccctggtcaccgtctcctca

SEQ ID NO: 230 - protein, 119 aa, synthetic construct
  REGION 1..119: note = synthetic
QVQLVESGGGVVQPGRSLRLSCAASGFTFSSYGMHWVRQAPVKGLEWVAFISYDGSNKYYADSVKGRFTISRDNSKNTLYLQMNSLTAEDTAVYYCAKESILAALFDYWGQGTLVTVSS

SEQ ID NO: 231 - DNA, 24 bp, synthetic construct
  misc_feature 1..24: note = synthetic
  source 1..24: mol_type = other DNA
ggattcaccttcagtagctatggc

SEQ ID NO: 232 - protein, 8 aa, synthetic construct
  REGION 1..8: note = synthetic
GFTFSSYG

SEQ ID NO: 233 - DNA, 24 bp, synthetic construct
  misc_feature 1..24: note = synthetic
  source 1..24: mol_type = other DNA
atatcatatgatggaagtaataaa

SEQ ID NO: 234 - protein, 8 aa, synthetic construct
  REGION 1..8: note = synthetic
ISYDGSNK

SEQ ID NO: 235 - DNA, 36 bp, synthetic construct; identical to SEQ ID NO: 171
  misc_feature 1..36: note = synthetic
  source 1..36: mol_type = other DNA

SEQ ID NO: 236 - protein, 12 aa, synthetic construct
  REGION 1..12: note = synthetic
AKESILAALFDY

SEQ ID NO: 237 - DNA, 336 bp, synthetic construct
  misc_feature 1..336: note = synthetic
  source 1..336: mol_type = other DNA
gacatcgtgatgacccagtctccagactccctggctgtgtctctgggcgagagggccaccatcaactgcaagtccagccagagtgttttatacagttccaacaataagaactacttagcttggtaccagcagaaaccaagacagcctcctaagctgctcatttactgggcatctattcgggaatccggggtccctgaccgattcagtggcagcgggtctgggacagatttcattctcaccatcagcagcctgcaggctgaagatgtggcagtttattactgtcagcaattttatagtattcccacttttggccaggggaccaagctggagatcaaa

SEQ ID NO: 238 - protein, 112 aa, synthetic construct
  REGION 1..112: note = synthetic
DIVMTQSPDSLAVSLGERATINCKSSQSVLYSSNNKNYLAWYQQKPRQPPKLLIYWASIRESGVPDRFSGSGSGTDFILTISSLQAEDVAVYYCQQFYSIPTFGQGTKLEIK

SEQ ID NO: 239 - DNA, 36 bp, synthetic construct
  misc_feature 1..36: note = synthetic
  source 1..36: mol_type = other DNA
cagagtgttttatacagttccaacaataagaactac

SEQ ID NO: 240 - protein, 12 aa, synthetic construct
  REGION 1..12: note = synthetic
QSVLYSSNNKNY

SEQ ID NO: 241-242 - skipped (2 entries)

SEQ ID NO: 243 - DNA, 24 bp, synthetic construct
  misc_feature 1..24: note = synthetic
  source 1..24: mol_type = other DNA
cagcaattttatagtattcccact

SEQ ID NO: 244 - protein, 8 aa, synthetic construct
  REGION 1..8: note = synthetic
QQFYSIPT

SEQ ID NO: 245 - DNA, 357 bp, synthetic construct
  misc_feature 1..357: note = synthetic
  source 1..357: mol_type = other DNA
caggtgcagctggtggagtctgggggaggcgtggtccagcctgggaggtccctgagactctcctgtgcagcctctggattcacctttagtagctatggcatgcactgggtccgccaggctccagtcaaggggctggagtgggtggcatttatatcaaatgataaaagtaataaatattatgcagactccttgaagggccgattcaccatctccagagacaattccaagaacacgctgtatctgcaaatgaacagcctgacagctgaagacacggctgtttattactgtgcgaaagagtccattttagcagccctctttgactattggggccagggaaccctggtcaccgtctcctca

SEQ ID NO: 246 - protein, 119 aa, synthetic construct
  REGION 1..119: note = synthetic
QVQLVESGGGVVQPGRSLRLSCAASGFTFSSYGMHWVRQAPVKGLEWVAFISNDKSNKYYADSLKGRFTISRDNSKNTLYLQMNSLTAEDTAVYYCAKESILAALFDYWGQGTLVTVSS

SEQ ID NO: 247 - DNA, 24 bp, synthetic construct
  misc_feature 1..24: note = synthetic
  source 1..24: mol_type = other DNA
ggattcacctttagtagctatggc

SEQ ID NO: 248 - protein, 8 aa, synthetic construct
  REGION 1..8: note = synthetic
GFTFSSYG

SEQ ID NO: 249 - DNA, 24 bp, synthetic construct
  misc_feature 1..24: note = synthetic
  source 1..24: mol_type = other DNA
atatcaaatgataaaagtaataaa

SEQ ID NO: 250 - protein, 8 aa, synthetic construct
  REGION 1..8: note = synthetic
ISNDKSNK

SEQ ID NO: 251 - DNA, 36 bp, synthetic construct
  misc_feature 1..36: note = synthetic
  source 1..36: mol_type = other DNA
gcgaaagagtccattttagcagccctctttgactat

SEQ ID NO: 252 - protein, 12 aa, synthetic construct
  REGION 1..12: note = synthetic
AKESILAALFDY

SEQ ID NO: 253 - DNA, 336 bp, synthetic construct
  misc_feature 1..336: note = synthetic
  source 1..336: mol_type = other DNA
gacatcgtgatgacccagtctccagactccctggctgtgtctctgggcgagagggccaccatcaactgcaagtccagccagagtgttttatacagctccaacaataagaactacttagcttggtaccagcagaaaccaagacagcctcctaagctactcatttactgggcatctattcgggaatccggggtccctgaccgattcagtggcagcgggtctgggacagatttcactctcaccatcagcagcctgcaggctgaagatgtggcagtttattactgtcaacaattttatagtgttcccacttttggccaggggaccaagctggagatcaaa

SEQ ID NO: 254 - protein, 112 aa, synthetic construct
  REGION 1..112: note = synthetic
DIVMTQSPDSLAVSLGERATINCKSSQSVLYSSNNKNYLAWYQQKPRQPPKLLIYWASIRESGVPDRFSGSGSGTDFTLTISSLQAEDVAVYYCQQFYSVPTFGQGTKLEIK

SEQ ID NO: 255 - DNA, 36 bp, synthetic construct; identical to SEQ ID NO: 207
  misc_feature 1..36: note = synthetic
  source 1..36: mol_type = other DNA

SEQ ID NO: 256 - protein, 12 aa, synthetic construct
  REGION 1..12: note = synthetic
QSVLYSSNNKNY

SEQ ID NO: 257-258 - skipped (2 entries)

SEQ ID NO: 259 - DNA, 24 bp, synthetic construct
  misc_feature 1..24: note = synthetic
  source 1..24: mol_type = other DNA
caacaattttatagtgttcccact

SEQ ID NO: 260 - protein, 8 aa, synthetic construct
  REGION 1..8: note = synthetic
QQFYSVPT

SEQ ID NO: 261 - DNA, 357 bp, synthetic construct
  misc_feature 1..357: note = synthetic
  source 1..357: mol_type = other DNA
caggtgcagctggtggagtctgggggaggcgtggtccagcctgggaggtccctgagactctcctgtgcagcctctggattcaccttcagtagctatggcatgcactgggtccgccaggctccagtcaaggggctggagtgggtggcatttatatcatttgatggaagtaataaatactatacagactccgtgaagggccgattcaccatctccagagacaattccaagaacacgctgtatctccaaatgaacagcctgacagctgaggacacggctatttattactgtgcgaaagagtccattttagcagccctctttgactactggggccagggaaccctggtcactgtctcctca

SEQ ID NO: 262 - protein, 119 aa, synthetic construct
  REGION 1..119: note = synthetic
QVQLVESGGGVVQPGRSLRLSCAASGFTFSSYGMHWVRQAPVKGLEWVAFISFDGSNKYYTDSVKGRFTISRDNSKNTLYLQMNSLTAEDTAIYYCAKESILAALFDYWGQGTLVTVSS

SEQ ID NO: 263 - DNA, 24 bp, synthetic construct
  misc_feature 1..24: note = synthetic
  source 1..24: mol_type = other DNA
ggattcaccttcagtagctatggc

SEQ ID NO: 264 - protein, 8 aa, synthetic construct
  REGION 1..8: note = synthetic
GFTFSSYG

SEQ ID NO: 265 - DNA, 24 bp, synthetic construct
  misc_feature 1..24: note = synthetic
  source 1..24: mol_type = other DNA
atatcatttgatggaagtaataaa

SEQ ID NO: 266 - protein, 8 aa, synthetic construct
  REGION 1..8: note = synthetic
ISFDGSNK

SEQ ID NO: 267 - DNA, 36 bp, synthetic construct; identical to SEQ ID NO: 171
  misc_feature 1..36: note = synthetic
  source 1..36: mol_type = other DNA

SEQ ID NO: 268 - protein, 12 aa, synthetic construct
  REGION 1..12: note = synthetic
AKESILAALFDY

SEQ ID NO: 269 - DNA, 336 bp, synthetic construct
  misc_feature 1..336: note = synthetic
  source 1..336: mol_type = other DNA
gacatcgtgatgacccagtctccagactccctggctgtgtctctgggcgagagggccaccatcaactgcaagtccagccagagtgttttatacagctccaacaataagaactacttagcttggtaccagcagaaaccaagacagcctcctaacctgctcatttactgggcatctattcgggaatccggggtccctgaccgattcagtggcagcgggtctgggacagatttcactctcaccatcagcagcctgcaggctgaagatgtggcattttattactgtcagcaattttatagtattcccacttttggccaggggaccaagctggagatcaaa

SEQ ID NO: 270 - protein, 112 aa, synthetic construct
  REGION 1..112: note = synthetic
DIVMTQSPDSLAVSLGERATINCKSSQSVLYSSNNKNYLAWYQQKPRQPPNLLIYWASIRESGVPDRFSGSGSGTDFTLTISSLQAEDVAFYYCQQFYSIPTFGQGTKLEIK

SEQ ID NO: 271 - DNA, 36 bp, synthetic construct; identical to SEQ ID NO: 207
  misc_feature 1..36: note = synthetic
  source 1..36: mol_type = other DNA

SEQ ID NO: 272 - protein, 12 aa, synthetic construct
  REGION 1..12: note = synthetic
QSVLYSSNNKNY

SEQ ID NO: 273-274 - skipped (2 entries)

SEQ ID NO: 275 - DNA, 24 bp, synthetic construct
  misc_feature 1..24: note = synthetic
  source 1..24: mol_type = other DNA
cagcaattttatagtattcccact

SEQ ID NO: 276 - protein, 8 aa, synthetic construct
  REGION 1..8: note = synthetic
QQFYSIPT

SEQ ID NO: 277 - DNA, 357 bp, synthetic construct
  misc_feature 1..357: note = synthetic
  source 1..357: mol_type = other DNA
caggtgcagctggtggagtctgggggaggcgtggtccagcctgggaggtccctgagactctcctgtgcagcctctggattcacctttaggacctatggcatgcactgggtccgccaggctccagtcaaggggctggagtgggtggcatttatatcaaaggatggaagtgataaatactatgtagactccgtgaagggccgattcaccatctccagagacaattccaagaacacgctgtttctgcaaatgaacagcctgacagctgaggacacggctgtttattattgtgcgaaagagtccattttagcagccctctttgactactggggccagggaaccctggtcactgtctcctca

SEQ ID NO: 278 - protein, 119 aa, synthetic construct
  REGION 1..119: note = synthetic
QVQLVESGGGVVQPGRSLRLSCAASGFTFRTYGMHWVRQAPVKGLEWVAFISKDGSDKYYVDSVKGRFTISRDNSKNTLFLQMNSLTAEDTAVYYCAKESILAALFDYWGQGTLVTVSS

SEQ ID NO: 279 - DNA, 24 bp, synthetic construct
  misc_feature 1..24: note = synthetic
  source 1..24: mol_type = other DNA
ggattcacctttaggacctatggc

SEQ ID NO: 280 - protein, 8 aa, synthetic construct
  REGION 1..8: note = synthetic
GFTFRTYG

SEQ ID NO: 281 - DNA, 24 bp, synthetic construct
  misc_feature 1..24: note = synthetic
  source 1..24: mol_type = other DNA
atatcaaaggatggaagtgataaa

SEQ ID NO: 282 - protein, 8 aa, synthetic construct
  REGION 1..8: note = synthetic
ISKDGSDK

SEQ ID NO: 283 - DNA, 36 bp, synthetic construct; identical to SEQ ID NO: 171
  misc_feature 1..36: note = synthetic
  source 1..36: mol_type = other DNA

SEQ ID NO: 284 - protein, 12 aa, synthetic construct
  REGION 1..12: note = synthetic
AKESILAALFDY

SEQ ID NO: 285 - DNA, 336 bp, synthetic construct
  misc_feature 1..336: note = synthetic
  source 1..336: mol_type = other DNA
gacatcgtgatgacccagtctccagactccctggctgtgtctctgggcgagagggccaccatcaactgcaagtccagccagagtgttttatacagctccaacaataagaactacttagcttggtaccagcagaaaccaagacagcctcctaaactcctcatttactgggcatctaatcgggaatccggggtccctgaccgattcagtggcagcgggtctgggacagatttcactctcaccatcagcagcctgcaggctgaagatgtggcagtttattactgtcagcaattttatagtgttcccacttttggccaggggaccaagctggagatcaaa

SEQ ID NO: 286 - protein, 112 aa, synthetic construct
  REGION 1..112: note = synthetic
DIVMTQSPDSLAVSLGERATINCKSSQSVLYSSNNKNYLAWYQQKPRQPPKLLIYWASNRESGVPDRFSGSGSGTDFTLTISSLQAEDVAVYYCQQFYSVPTFGQGTKLEIK

SEQ ID NO: 287 - DNA, 36 bp, synthetic construct; identical to SEQ ID NO: 207
  misc_feature 1..36: note = synthetic
  source 1..36: mol_type = other DNA

SEQ ID NO: 288 - protein, 12 aa, synthetic construct
  REGION 1..12: note = synthetic
QSVLYSSNNKNY

SEQ ID NO: 289-290 - skipped (2 entries)

SEQ ID NO: 291 - DNA, 24 bp, synthetic construct
  misc_feature 1..24: note = synthetic
  source 1..24: mol_type = other DNA
cagcaattttatagtgttcccact

SEQ ID NO: 292 - protein, 8 aa, synthetic construct
  REGION 1..8: note = synthetic
QQFYSVPT

SEQ ID NO: 293 - DNA, 357 bp, synthetic construct
  misc_feature 1..357: note = synthetic
  source 1..357: mol_type = other DNA
caggtgcagctggtggagtctgggggaggcgtggtccagcctgggaggtccctgagactctcctgtgcagcctctggattcacctttagtagctatggcatgcactgggtccgccaggctccagtcaaggggctggagtgggtggcatttatatcaaatgataaaagtaataaatactatgcagactccgtgaagggccgattcaccatctccagagacaattccaagaacacgctgtatctgcaaatgaacagcctgacagctgaggacacggctgtttattactgtgcgaaagagtccattttagcagccctctttgactcctggggccagggaaccctggtcaccgtctcctca

SEQ ID NO: 294 - protein, 119 aa, synthetic construct
  REGION 1..119: note = synthetic
QVQLVESGGGVVQPGRSLRLSCAASGFTFSSYGMHWVRQAPVKGLEWVAFISNDKSNKYYADSVKGRFTISRDNSKNTLYLQMNSLTAEDTAVYYCAKESILAALFDSWGQGTLVTVSS

SEQ ID NO: 295 - DNA, 24 bp, synthetic construct
  misc_feature 1..24: note = synthetic
  source 1..24: mol_type = other DNA
ggattcacctttagtagctatggc

SEQ ID NO: 296 - protein, 8 aa, synthetic construct
  REGION 1..8: note = synthetic
GFTFSSYG

SEQ ID NO: 297 - DNA, 24 bp, synthetic construct
  misc_feature 1..24: note = synthetic
  source 1..24: mol_type = other DNA
atatcaaatgataaaagtaataaa

SEQ ID NO: 298 - protein, 8 aa, synthetic construct
  REGION 1..8: note = synthetic
ISNDKSNK

SEQ ID NO: 299 - DNA, 36 bp, synthetic construct
  misc_feature 1..36: note = synthetic
  source 1..36: mol_type = other DNA
gcgaaagagtccattttagcagccctctttgactcc

SEQ ID NO: 300 - protein, 12 aa, synthetic construct
  REGION 1..12: note = synthetic
AKESILAALFDS

SEQ ID NO: 301 - DNA, 336 bp, synthetic construct
  misc_feature 1..336: note = synthetic
  source 1..336: mol_type = other DNA
gacatcgtgatgacccagtctccagactccctggctgtgtctctgggcgagagggccaccatcaactgcaagtccagccagagtgttttatacagctccaacaataagaactacttagcttggtaccagcagaaaccaagacagcctcctaagctgctcatttactgggcatctattcgggaatccggggtccctgaccgattcagtggcagcgggtctggggcagatttcactctcaccatcagcagcctgcaggctgcagatgtggcagtttattactgtcagcaattttatagtgttcccacttttggccaggggaccaagctggagatcaaa

SEQ ID NO: 302 - protein, 112 aa, synthetic construct
  REGION 1..112: note = synthetic
DIVMTQSPDSLAVSLGERATINCKSSQSVLYSSNNKNYLAWYQQKPRQPPKLLIYWASIRESGVPDRFSGSGSGADFTLTISSLQAADVAVYYCQQFYSVPTFGQGTKLEIK

SEQ ID NO: 303 - DNA, 36 bp, synthetic construct; identical to SEQ ID NO: 207
  misc_feature 1..36: note = synthetic
  source 1..36: mol_type = other DNA

SEQ ID NO: 304 - protein, 12 aa, synthetic construct
  REGION 1..12: note = synthetic
QSVLYSSNNKNY

SEQ ID NO: 305-306 - skipped (2 entries)

SEQ ID NO: 307 - DNA, 24 bp, synthetic construct
  misc_feature 1..24: note = synthetic
  source 1..24: mol_type = other DNA
cagcaattttatagtgttcccact

SEQ ID NO: 308 - protein, 8 aa, synthetic construct
  REGION 1..8: note = synthetic
QQFYSVPT

(19) United States
(12) Patent Application Publication
(10) Pub. No.: US 2025/0101119 A1
(43) Pub. Date: Mar. 27, 2025

(54) METHODS OF TREATING UREA CYCLE DISORDERS BY INTERFERING WITH GLUCAGON RECEPTOR SIGNALING

(71) Applicant: Regeneron Pharmaceuticals, Inc., Tarrytown, NY (US)

(72) Inventors: Haruka Okamoto, New York, NY (US); Xiping Cheng, Northvale, NJ (US); Jesper Gromada, Concord, MA (US)

(21) Appl. No.: 18/906,101

(22) Filed: Oct. 3, 2024

Related U.S. Application Data
(62) Division of application No. 16/640,907, filed on Feb. 21, 2020, now Pat. No. 12,139,546.
(60) Provisional application No. 62/548,632, filed on Aug. 22, 2017.

(51) Int. Cl. C07K 16/28; A61K 31/7088; A61K 45/06

(52) U.S. Cl. CPC C07K 16/2869; A61K 31/7088; A61K 45/06

(57) ABSTRACT

Provided herein are methods of treating a subject with hyperammonemia or a urea cycle disorder. The methods comprise administering to a subject in need thereof a therapeutic amount of a glucagon signaling pathway inhibitor, such that ammonia levels are lowered or that amino acid metabolism enzymes are down-regulated, or a condition or disease characterized by hyperammonemia is mediated, or at least one symptom or complication associated with the condition or disease is alleviated or reduced in severity. The glucagon signaling pathway inhibitor can be a small molecule inhibitor of the signaling pathway, an antisense inhibitor of the signaling pathway, shRNA, siRNA, a GCG neutralizing monoclonal antibody, a GCGR antagonist, a peptide inhibitor of the signaling pathway, a DARPin, a Spiegelmer, an aptamer, engineered Fn type-III domains, etc. The therapeutic methods are useful for treating a human suffering from hyperammonemia or a urea cycle disorder.

This application is a divisional application of U.S. patent application Ser. No. 16/640,907, which is a 371 National Phase Patent Application of International Application Serial No. PCT/US2018/047286 filed on Aug. 21, 2018, which claims the benefit of U.S. Provisional Patent Application No. 62/548,632, filed Aug. 22, 2017, which is herein incorporated by reference in its entirety.

### TECHNICAL FIELD

The invention relates to methods of using a glucagon (GCG) inhibitor or a glucagon receptor (GCGR) antagonist to treat urea cycle disorders and/or hyperammonemia, and/or reducing the therapeutic dose of sodium phenylbutyrate or sodium benzoate in a subject in need thereof.

### SEQUENCE LISTING

An official copy of the sequence listing is submitted concurrently with the specification electronically via Patent Center. The content of the electronic sequence listing (10366US02_Sequence_Listing_ST26.xml; Size 368,640 bytes; and Date of Creation: Oct. 2, 2024) is herein incorporated by reference in its entirety.

### BACKGROUND

Glucagon is a 29 residue polypeptide hormone, which in cooperation with insulin, mediates homeostatic regulation of the amount of glucose in the blood. Glucagon primarily acts by stimulating certain cells, for example, liver cells, to release glucose when blood glucose levels fall to maintain normal blood glucose levels. The action of glucagon is opposite to that of insulin, which stimulates cells to take up and store glucose whenever blood glucose levels rise. Glucagon is produced in the alpha cells of the pancreas, whereas insulin is secreted from the neighboring beta cells. The glucagon receptor is a member of the class B G-protein coupled family of receptors, and is activated by glucagon binding. Glucagon receptors are predominantly expressed in the liver and kidney.

The action of glucagon can be suppressed by providing an antagonist, such as a small molecule inhibitor, shRNA, siRNA, a GCG antibody, or a GCGR antibody, as described herein. Anti-GCG antibodies are mentioned, e.g., in U.S. Pat. Nos. 4,206,199; 4,221,777; 4,423,034; 4,272,433; 4,407,965; 5,712,105; and in PCT publications WO2007/124463 and WO2013/081993. Anti-GCGR antibodies are described in U.S. Pat. Nos. 5,770,445, 7,947,809, and 8,545,847; European patent application EP2074149A2; EP patent EP0658200B1; US patent publications 2009/0041784; 2009/0252727; and 2011/0223160; and PCT publication WO2008/036341. Small molecule inhibitors of GCG or GCGR are mentioned, e.g. in WO 07/47676; WO 06/86488; WO 05/123688; WO 05/121097; WO 06/14618; WO 08/42223; WO 08/98244; WO 2010/98948; U.S. Pat. No. 20,110,306624; WO 2010/98994; WO 2010/88061; WO 2010/71750; WO 2010/30722; WO 06/104826; WO 05/65680; WO 06/102067; WO 06/17055; WO 2011/07722; or WO 09/140342. Inhibition of glucagon affects the GCG/GCGR downstream signaling pathway, inhibiting cAMP and PKA.

Urea cycle disorders (UCDs) result from genetic mutations causing defects in the metabolism of nitrogen produced by the breakdown of protein and other nitrogen-containing compounds. Deficiencies in N-acetylglutamate synthetase (NAGS), carbamoylphosphate synthetase I (CPSI), ornithine transcarbamylase (OTC), argininosuccinate synthetase (ASS, also referred to as Citrullinemia I), citrin (Citrullinemia II), argininosuccinate lyase (Argininosuccinic Aciduria), arginase (Hyperargininemia), and ornithine translocase (HHH Syndrome) are associated with the disease, though there are differences in timing of presentation of hyperammonemia crisis as some affect newborns, others affect adults, and some, for example, Citrullinemia II, can present in either newborns or adults. Amino acid metabolism enzymes are upstream of the urea cycle and are involved in ammonia production through degradation of amino acids. The enzymes involved include cystathionase (CTH), serine dehydratase (SDS), ornithine aminotransferase (OAT), and glutaminase 2 (GLS-2).

Urea cycle disorders and hyperammonemia cause a plethora of symptoms, but typically result in intellectual and developmental disabilities and eventual death without treatment. Available treatment options include dietary protein restriction and nitrogen disposal using phenylbutyrate, though ultimately many subjects with hyperammonemia and/or a urea cycle disorder require liver transplantation.

Given the absence of effective therapies to treat, or to slow the progression of severe hyperammonemia and/or urea cycle disorders, i.e., to extend the life and/or improve the quality of life of a subject having hyperammonemia and/or a urea cycle disorder, there is a need to identify and explore the use of other agents for treating these diseases, such as the glucagon signaling pathway inhibitors and antagonists as described herein.

### BRIEF SUMMARY

Provided herein are methods for treating a subject with a condition or disease characterized by hyperammonemia, by administering a glucagon signaling pathway antagonist, e.g. a GCG inhibitor or a GCGR antagonist, or a pharmaceutical composition comprising a GCG inhibitor or GCGR antagonist. A GCG inhibitor or GCGR antagonist is a compound capable of blocking or inhibiting the glucagon receptor signaling pathway. The antagonist may take the form of a small molecule inhibitor, shRNA, siRNA, peptide inhibitor, CRISPR technology (Clustered regularly interspaced short palindromic repeats; CRISPR technology can generate GCGR knock-down or deletion of regulatory sequences affecting GCGR activity), an antisense inhibitor, DARPin, and a GCG or GCGR neutralizing monoclonal antibody. The glucagon signaling pathway antagonist can be administered alone, in a pharmaceutical composition, or in conjunction with one or more therapeutic agents, supplements, or therapeutic procedures useful in treating a condition or disease associated with hyperammonemia, or in treating one or more symptoms associated with a urea cycle disorder, or in lowering blood ammonia levels in a subject having a condition or disease associated with a urea cycle disorder.

In some embodiments, the method comprises administering to a subject having hyperammonemia a therapeutically effective amount of a composition comprising a glucagon signaling pathway antagonist such that serum ammonia levels are lowered or that the condition or disease is mediated, or at least one symptom or complication associated with the condition or disease is alleviated or reduced in severity. In some aspects, the hyperammonemia is congenital hyperammonemia. For example, the congenital hyperammonemia can be caused by a defect in a urea cycle enzyme selected from the group consisting of carbamyl phosphate synthetase (CPS1), N-acetylglutamate synthetase (NAGS), ornithine transcarbamylase (OTC), argininosuccinic acid synthetase (ASS), argininosuccinate lyase (ASL), and arginase (AR1); or a defect in a urea cycle transporter, e.g. ornithine translocase (ORNT1) and citrin. The congenital hyperammonemia can be caused by methylmalonic aciduria, propionic aciduria, or isovaleric aciduria. The congenital hyperammonemia can be caused by medium-chain acyl-CoA dehydrogenase deficiency, multiple acyl-CoA dehydrogenase deficiency, carnitine palmitoyltransferase II deficiency, carnitine-acylcarnitine translocase, lysinuric protein intolerance, pyrroline-5-carboxylate synthetase deficiency, pyruvate carboxylase deficiency, ornithine aminotransferase deficiency, carbonic anhydrase Va deficiency, hyperinsulinism-hyperammonemia syndrome, mitochondrial disorders, and glutamine synthetase deficiency. In some aspects, the hyperammonemia is acquired. For example, the hyperammonemia can be caused by liver disease and complications thereof; by treatment with a therapeutic agent (L-asparaginase or pegaspargase), 5-pentanoic acid, valproic acid, a corticosteroid, or a cyclophosphamide.

In some aspects, the hyperammonemia is caused by herpes simplex infection, hepatitis B infection, or infection with urease-producing organisms.

In some aspects, the hyperammonemia is caused by total parenteral nutrition (with relative arginine deficiency), L-asparaginase treatment, nutritional carnitine deficiency, cystoscopy with glycine-containing solutions, post-lung/bone marrow transplantation, vascular malformations, or transient hyperammonemia of the newborn.

Provided herein are methods for treating a subject with a urea cycle disorder, wherein the subject exhibits elevated levels of ammonia. In some embodiments, the method comprises administering to the subject a therapeutically effective amount of a composition comprising a glucagon signaling pathway antagonist.

Provided herein are methods for treating a subject with a urea cycle disorder, wherein the subject does not exhibit elevated levels of ammonia. In some embodiments, the method comprises administering to the subject a therapeutically effective amount of a composition comprising a glucagon signaling pathway antagonist.

Provided herein are methods for lowering blood ammonia levels, or for treating a condition or disease associated with, or characterized in part by hyperammonemia, or at least one symptom or complication associated with the condition or disease. In some embodiments, the method comprises administering to a subject a therapeutically effective amount of a composition comprising glucagon signaling pathway antagonist, such that blood ammonia levels are lowered or that the condition or disease is mediated, or at least one symptom or complication associated with the condition or disease is alleviated or reduced in severity.

In some aspects, the glucagon signaling pathway antagonist is selected from a small molecule inhibitor, shRNA, siRNA, a peptide inhibitor, CRISPR technology (Clustered regularly interspaced short palindromic repeats; CRISPR technology can generate GCGR knock-down or deletion of regulatory sequences affecting GCGR activity), an antisense inhibitor, a DARPin, and a GCG inhibitor or a GCGR antagonist (such as a neutralizing monoclonal antibody).

In some aspects, the GCGR antagonist can be an anti-GCGR antibody. The anti-GCGR antibody can inhibit or antagonize the GCGR. The anti-GCGR antibody can inhibit or block the GCGR signaling pathway. In some aspects, the GCG inhibitor can be an anti-GCG antibody. The anti-GCG antibody can inhibit binding of GCG to the GCGR.

In certain embodiments, the antibody or antigen-binding fragment specifically binds hGCGR, and comprises the heavy and light chain CDR domains contained within heavy and light chain sequence pairs selected from the group consisting of SEQ ID NO: 2/10, 18/26, 34/42, 50/58, 66/68, 70/78, 86/88, 90/98, 106/108, 110/118, 126/128, 130/138 and 146/148.

In certain embodiments, the antibody or antigen-binding fragment comprises the heavy and light chain CDR domains contained within the HCVR/LCVR amino acid sequence pair of SEQ ID NOs: 86/88.

In certain embodiments, the antibody or antigen-binding fragment comprises a HCVR/LCVR amino acid sequence pair of SEQ ID NOs: 86/88.

In one embodiment, the human antibody or antigen-binding fragment of a human antibody that binds hGCGR, comprises a heavy chain variable region (HCVR) having an amino acid sequence selected from the group consisting of SEQ ID NO: 2, 18, 34, 50, 66, 70, 86, 90, 106, 110, 126, 130 and 146, or a substantially similar sequence thereof having at least 90%, at least 95%, at least 98% or at least 99% sequence identity.

In one embodiment, the human antibody or antigen-binding fragment of a human antibody that binds hGCGR comprises a light chain variable region (LCVR) having an amino acid sequence selected from the group consisting of SEQ ID NO: 10, 26, 42, 58, 68, 78, 88, 98, 108, 118, 128, 138 and 148, or a substantially similar sequence thereof having at least 90%, at least 95%, at least 98% or at least 99% sequence identity.

In certain embodiments, the human antibody or fragment thereof that binds hGCGR comprises a HCVR/LCVR amino acid sequence pair selected from the group consisting of SEQ ID NO: 2/10, 18/26, 34/42, 50/58, 66/68, 70/78, 86/88, 90/98, 106/108, 110/118, 126/128, 130/138, and 146/148. In certain embodiments, the HCVR/LCVR amino acid sequence pair is selected from the group consisting of SEQ ID NO: 34/42, 70/78, 86/88, 110/118 and 126/128.

In certain embodiments, the isolated human antibody or an antigen-binding fragment thereof that binds specifically to hGCGR comprises a HCVR comprising the three heavy chain CDRs (HCDR1, HCDR2 and HCDR3) contained within the HCVR sequence selected from the group consisting of SEQ ID NO: 2, 18, 34, 50, 66, 70, 86, 90, 106, 110, 126, 130 and 146; and/or a LCVR comprising the three light chain CDRs (LCDR1, LCDR2 and LCDR3) contained within the LCVR sequences selected from the group consisting of SEQ ID NO: 10, 26, 42, 58, 68, 78, 88, 98, 108, 118, 128, 138 and 148.

In certain embodiments, the methods provided herein contemplate the use of an isolated human antibody or antigen-binding fragment thereof that binds hGCGR comprising a HCDR3 domain having an amino acid sequence selected from the group consisting of SEQ ID NO: 8, 24, 40, 56, 76, 96, 116 and 136, or a substantially similar sequence thereof having at least 90%, at least 95%, at least 98% or at least 99% sequence identity; and/or a LCDR3 domain having an amino acid sequence selected from the group consisting of SEQ ID NO: 16, 32, 48, 64, 84, 104, 124 and 144, or a substantially similar sequence thereof having at least 90%, at least 95%, at least 98% or at least 99% sequence identity.

In one embodiment, the methods provided herein contemplate use of an antibody or fragment thereof that further comprises a HCDR1 domain having an amino acid sequence selected from the group consisting of SEQ ID NO: 4, 20, 36, 52, 72, 92, 112 and 132, or a substantially similar sequence thereof having at least 90%, at least 95%, at least 98% or at least 99% sequence identity; a HCDR2 domain having an amino acid sequence selected from the group consisting of SEQ ID NO: 6, 22, 38, 54, 74, 94, 114 and 134, or a substantially similar sequence thereof having at least 90%, at least 95%, at least 98% or at least 99% sequence identity; a LCDR1 domain having an amino acid sequence selected from the group consisting of SEQ ID NO: 12, 28, 44, 60, 80, 100, 120 and 140, or a substantially similar sequence thereof having at least 90%, at least 95%, at least 98% or at least 99% sequence identity; and a LCDR2domain having an amino acid sequence selected from the group consisting of SEQ ID NO: 14, 30, 46, 62, 82, 102, 122 and 142, or a substantially similar sequence thereof having at least 90%, at least 95%, at least 98% or at least 99% sequence identity.

In one embodiment, the antibody or antigen-binding fragment of an antibody comprises:
(a) a HCDR3 domain having an amino acid sequence selected from the group consisting of SEQ ID NO: 8, 24, 40, 56, 76, 96, 116 and 136; and
(b) a LCDR3 domain having an amino acid sequence selected from the group consisting of SEQ ID NO: 16, 32, 48, 64, 84, 104, 124 and 144.

In a related embodiment, the antibody or antigen-binding fragment of the antibody further comprises:
(c) a HCDR1 domain having an amino acid sequence selected from the group consisting of SEQ ID NO: 4, 20, 36, 52, 72, 92, 112 and 132;
(d) a HCDR2 domain having an amino acid sequence selected from the group consisting of SEQ ID NO: 6, 22, 38, 54, 74, 94, 114 and 134;
(e) a LCDR1 domain having an amino acid sequence selected from the group consisting of SEQ ID NO: 12, 28, 44, 60, 80, 100, 120 and 140; and
(f) a LCDR2 domain having an amino acid sequence selected from the group consisting of SEQ ID NO: 14, 30, 46, 62, 82, 102, 122 and 142.

In one embodiment, the antibody or antigen-binding fragment thereof comprises a HCVR comprising a HCDR1 domain having an amino acid sequence selected from one of SEQ ID NO: 4, 20, 36, 52, 72, 92, 112 and 132; a HCDR2 domain having an amino acid sequence selected from one of SEQ ID NO: 6, 22, 38, 54, 74, 94, 114 and 134; a HCDR3 domain having an amino acid sequence selected from one of SEQ ID NOs: 8, 24, 40, 56, 76, 96, 116 and 136; and a LCVR comprising a LCDR1 domain having an amino acid sequence selected from one of SEQ ID NO: 12, 28, 44, 60, 80, 100, 120 and 140; a LCDR2 domain having an amino acid sequence selected from one of SEQ ID NO: 14, 30, 46, 62, 82, 102, 122 and 142; and a LCDR3 domain having an amino acid sequence selected from one of SEQ ID NO: 16, 32, 48, 64, 84, 104, 124 and 144.

In certain embodiments, the human antibody or antigen-binding fragment of a human antibody that binds to human GCGR comprises a HCDR3/LCDR3 amino acid sequence pair selected from the group consisting of SEQ ID NO: 8/16, 24/32, 40/48, 56/64, 76/84, 86/88, 96/104, 116/124 and 136/144. Non-limiting examples of anti-GCGR antibodies having these HCDR3/LCDR3 pairs are the antibodies designated H4H1345N, H4H1617N, H4H1765N, H4H1321B and H4H1321P, H4H1327B and H4H1327P, H4H1328B and H4H1328P, H4H1331B and H4H1331P, H4H1339B and H4H1339P, respectively.

In one embodiment, the isolated antibody or antigen-binding fragment thereof useful according to the methods provided herein, that specifically binds to GCG and neutralizes at least one activity associated with GCG, comprises: (a) three heavy chain complementarity determining regions (HCDR1, HCDR2 and HCDR3) contained within a heavy chain variable region (HCVR) amino acid sequence selected from the group consisting of SEQ ID NOs: 150, 166, 182, 198, 214, 230, 246, 262, 278 and 294; and (b) three light chain CDRs (LCDR1, LCDR2 and LCDR3) contained within a light chain variable region (LCVR) amino acid sequence selected from the group consisting of SEQ ID NOs: 158, 174, 190, 206, 222, 238, 254, 270, 286 and 302.

In some embodiments, the isolated antibody or antigen-binding fragment thereof that specifically binds to GCG and neutralizes at least one activity associated with GCG, comprises an HCVR having an amino acid sequence selected from the group consisting of SEQ ID NOs: 150, 166, 182, 198, 214, 230, 246, 262, 278 and 294 and a LCVR having an amino acid sequence selected from the group consisting of SEQ ID NOs: 158, 174, 190, 206, 222, 238, 254, 270, 286 and 302.

In some embodiments, the isolated antibody or antigen-binding fragment thereof that specifically binds to GCG and neutralizes at least one activity associated with GCG, comprises a HCVR/LCVR amino acid sequence pair selected from the group consisting of SEQ ID NOs: 150/158; 166/174; 182/190; 198/206; 214/222; 230/238; 246/254; 262/270; 278/286 and 294/302.

In some embodiments, the HCVR/LCVR amino acid sequence pair comprises SEQ ID NOs: 166/174.

In some embodiments, the HCVR/LCVR amino acid sequence pair comprises SEQ ID NOs: 182/190.

In one embodiment, the isolated antibody or antigen-binding fragment thereof useful according to the methods provided herein, comprises:
(a) a HCDR1 domain having an amino acid sequence selected from the group consisting of SEQ ID NOs: 152, 168, 184, 200, 216, 232, 248, 264, 280, and 296;
(b) a HCDR2 domain having an amino acid sequence selected from the group consisting of SEQ ID NOs: 154, 170, 186, 202, 218, 234, 250, 266, 282, and 298;
(c) a HCDR3 domain having an amino acid sequence selected from the group consisting of SEQ ID NOs: 156, 172, 188, 204, 220, 236, 252, 268, 284, and 300;
(d) a LCDR1 domain having an amino acid sequence selected from the group consisting of SEQ ID NOs: 160, 176, 192, 208, 224, 240, 256, 272, 288, and 304;
(e) a LCDR2 domain having an amino acid sequence selected from the group consisting of SEQ ID NOs: 162, 178, 194, 210, 226, 242, 258, 274, 290, and 306; and
(f) a LCDR3 domain having an amino acid sequence selected from the group consisting of SEQ ID NOs: 164, 180, 196, 212, 228, 244, 260, 276, 292, and 308.

In one embodiment, the isolated antibody or antigen-binding fragment thereof useful according to the methods provided herein, comprises:
(a) a HCDR1 domain comprising the amino acid sequence of SEQ ID NO: 168;
(b) a HCDR2 domain comprising the amino acid sequence of SEQ ID NO: 170;
(c) a HCDR3 domain comprising the amino acid sequence of SEQ ID NO: 172;
(d) a LCDR1 domain comprising the amino acid sequence of SEQ ID NO: 176;
(e) a LCDR2 domain comprising the amino acid sequence of SEQ ID NO: 178; and
(f) a LCDR3 domain comprising the amino acid sequence of SEQ ID NO: 180.

In one embodiment, the isolated antibody or antigen-binding fragment thereof useful according to the methods provided herein, comprises:
(a) a HCDR1 domain comprising the amino acid sequence of SEQ ID NO: 184;
(b) a HCDR2 domain comprising the amino acid sequence of SEQ ID NO: 186;
(c) a HCDR3 domain comprising the amino acid sequence of SEQ ID NO: 188;
(d) a LCDR1 domain comprising the amino acid sequence of SEQ ID NO: 192;
(e) a LCDR2 domain comprising the amino acid sequence of SEQ ID NO: 194; and
(f) a LCDR3 domain comprising the amino acid sequence of SEQ ID NO: 196.

Also useful according to the methods provided herein are antibodies or antigen-binding fragments thereof that specifically bind GCG, comprising a heavy chain CDR1 (HCDR1) comprising an amino acid sequence selected from any of the HCDR1 amino acid sequences provided herein or a substantially similar sequence thereof having at least 90%, at least 95%, at least 98% or at least 99% sequence identity.

Also useful according to the methods provided herein are antibodies or antigen-binding fragments thereof that specifically bind GCG, comprising a heavy chain CDR2 (HCDR2) comprising an amino acid sequence selected from any of the HCDR2 amino acid sequences provided herein or a substantially similar sequence thereof having at least 90%, at least 95%, at least 98% or at least 99% sequence identity.

Also useful according to the methods provided herein are antibodies or antigen-binding fragments thereof that specifically bind GCG, comprising a heavy chain CDR3 (HCDR3) comprising an amino acid sequence selected from any of the HCDR3 amino acid sequences provided herein or a substantially similar sequence thereof having at least 90%, at least 95%, at least 98% or at least 99% sequence identity.

Also useful according to the methods provided herein are antibodies or antigen-binding fragments thereof that specifically bind GCG, comprising a light chain CDR1 (LCDR1) comprising an amino acid sequence selected from any of the LCDR1 amino acid sequences provided herein or a substantially similar sequence thereof having at least 90%, at least 95%, at least 98% or at least 99% sequence identity.

Also useful according to the methods provided herein are antibodies or antigen-binding fragments thereof that specifically bind GCG, comprising a light chain CDR2 (LCDR2) comprising an amino acid sequence selected from any of the LCDR2 amino acid sequences provided herein or a substantially similar sequence thereof having at least 90%, at least 95%, at least 98% or at least 99% sequence identity.

Also useful according to the methods provided herein are antibodies or antigen-binding fragments thereof that specifically bind GCG, comprising a light chain CDR3 (LCDR3) comprising an amino acid sequence selected from any of the LCDR3 amino acid sequences listed herein or a substantially similar sequence thereof having at least 90%, at least 95%, at least 98% or at least 99% sequence identity.

Also useful according to the methods provided herein are antibodies or antigen-binding fragments thereof that specifically bind GCG, comprising an HCDR3 and an LCDR3 amino acid sequence pair (HCDR3/LCDR3) comprising any of the HCDR3 amino acid provided herein paired with any of the LCDR3 amino acid sequences provided herein. According to certain embodiments, the antibodies, or antigen-binding fragments thereof, comprise an HCDR3/LCDR3 amino acid sequence pair contained within any of the exemplary anti-GCG antibodies provided herein. In certain embodiments, the HCDR3/LCDR3 amino acid sequence pair comprises SEQ ID NOs: 172/180.

Also useful according to the methods provided herein are antibodies or antigen-binding fragments thereof that specifically bind GCG, comprising a set of six CDRs (i.e., HCDR1-HCDR2-HCDR3-LCDR1-LCDR2-LCDR3) contained within any of the exemplary anti-GCG antibodies provided herein. In certain embodiments, the HCDR1/HCDR2/HCDR3/LCDR1/LCDR2/LCDR3 amino acid sequence set comprises SEQ ID NOs: 168/170/172/176/178/180. In certain embodiments, the HCDR1/HCDR2/HCDR3/LCDR1/LCDR2/LCDR3 amino acid sequence set comprises SEQ ID NOs: 184/186/188/192/194/196.

In a related embodiment, the antibodies, or antigen-binding fragments thereof that specifically bind GCG, comprise a set of six CDRs (i.e.,

HCDR1/HCDR2/HCDR3/LCDR1/LCDR2/LCDR3) contained within an HCVR/LCVR amino acid sequence pair as defined by any of the exemplary anti-GCG antibodies provided herein. For example, the antibodies or antigen-binding fragments thereof that specifically bind GCG, comprise the HCDR1/HCDR2/HCDR3/LCDR1/LCDR2/LCDR3 amino acid sequences set contained within an HCVR/LCVR amino acid sequence pair selected from the group consisting of: 166/174; 182/190; 198/206; 214/222; 230/238; 246/254; 262/270; 278/286 and 294/302.

Non-limiting examples of antibodies that specifically bind GCG and comprise the CDR sequences provided above, include HIH059P, H4H10223P, H4H10231P, H4H10232P, H4H10236P, H4H10237P, H4H10238P, H4H10250P, H4H10256P, and H4H10270P.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, (1991) “Sequences of Proteins of Immunological Interest,” National Institutes of Health, Bethesda, Md.; Al-Lazikani et al., (1997) J. Mol. Biol. 273:927-948; and Martin et al., (1989) Proc. Natl. Acad. Sci. USA 86:9268-9272. Public databases are also available for identifying CDR sequences within an antibody.

In some embodiments, a subject having hyperammonemia may suffer from one of the conditions or diseases selected from the following: a urea cycle disorder, liver disease and conditions associated with liver disease such as hepatic encephalopathy and fetor hepaticus, and a condition or disease associated with the presence of an enzyme defect associated with a urea cycle disorder or reported to cause hyperammonemia. In some embodiments, elevated ammonia levels are detected in subject sera. In some embodiments, elevated glutamine levels are detected in subject sera.

In some aspects, the enzyme defect associated with a urea cycle disorder is selected from the following: carbamyl phosphate synthetase (CPS1), N-acetylglutamate synthetase (NAGS), ornithine transcarbamylase (OTC), argininosuccinic acid synthetase (ASS), argininosuccinate lyase (ASL), and arginase (AR1). In some aspects, the urea cycle disorder is related to a transporter defect selected from the following: ornithine translocase (ORNT1; ornithine/citrulline carrier; solute carrier family 25, member 15) and citrin (aspartate/glutamate carrier; solute carrier family 25, member 13).

In some aspects, the glucagon signaling pathway antagonist is administered with amino acid formulas, such as those selected from Cyclinex (e.g., Cyclinex-1 or-2), EAA (essential amino acids), UCD I or II (Urea Cycle Disorder-1 or-II), and individual branched chain amino acids.

In some aspects, the glucagon signaling pathway antagonist is administered with antioxidants or electrolytes.

In some aspects, the glucagon signaling pathway antagonist is administered with L-citrulline or L-arginine free base.

In some aspects, the glucagon signaling pathway antagonist is administered along with hemodialysis or continuous renal replacement.

In some aspects, the composition comprising the glucagon signaling pathway antagonist is administered to a subject in combination with at least one additional therapeutic agent. The additional therapeutic agent can be any agent that alleviates or reduces the symptoms and signs associated with hyperammonemia and/or a urea cycle disorder. In some embodiments, at least one additional therapeutic agent is selected from the following: non-absorbable antibiotic (rifaximin or lactulose), sodium phenylbutyrate, sodium benzoate, sodium phenylacetate, glycerol phenylbutyrate, carbamyl glutamate (Carbaglu®), a second GCG inhibitor, and a second GCGR antagonist.

Provided herein are methods of reducing the amount and/or dosage of sodium phenylbutyrate or sodium benzoate necessary to treat a subject with hyperammonemia. In some embodiments, the method comprises administering to the subject a therapeutically effective amount of a composition comprising a glucagon signaling pathway antagonist. In some aspects, the glucagon signaling pathway antagonist is administered concomitantly with sodium phenylbutyrate or sodium benzoate.

Other objects and advantages will become apparent from a review of the ensuing detailed description.

### BRIEF DESCRIPTION OF THE FIGURES

FIG. 1 is a graphical representation of Table 3, and depicts mean±SEM of non-fasting blood glucose levels for the four groups of mice.

FIG. 2 is a graphical representation of Table 4, and depicts mean±SEM of body weight changes from baseline for the four groups of mice.

FIG. 3 is a graphical representation of Table 5, and depicts mean±SEM plasma ammonia levels for the four groups of mice at baseline and weeks 2, 3, 4, 6, 7, and 8.

FIG. 4 depicts survival curves for the four groups of mice.

FIG. 5 shows the plasma ammonia levels over time of wild-type and Otc mutant mice on a 30% protein diet treated with isotype control or H4H1327P antibody. **: p<0.01 between the two treatments in Otc mutant mice, ***: p<0.001 between the two treatments in Otc mutant mice, ****: p<0.0001 between the two treatments in Otc mutant mice.

FIG. 6 depicts survival curves for wild-type and Otc mutant mice on a 30% protein diet treated with isotype control or H4H1327P antibody.

FIG. 7 shows body weight changes over time from baseline of wild-type and Otc mutant mice on a 30% protein diet treated with isotype control or H4H1327P antibody. *: p<0.05 between the two treatments in Otc mutant mice, ****: p<0.0001 between the two treatments in Otc mutant mice.

FIG. 8 shows blood glucose levels (mg/dL) over time of wild-type and Otc mutant mice on a 30% protein diet treated with isotype control or H4H1327P antibody. *: p<0.05 between the two treatments in Otc mutant mice, ***: p<0.001 between the two treatments in Otc mutant mice, ****: p<0.0001 between the two treatments in Otc mutant mice, \hat{ }\hat{ }\hat{ }\hat{ }: p<0.0001 between the two treatments in wild-type mice.

### DESCRIPTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All patents, applications and non-patent publications mentioned in this specification are incorporated herein by reference in their entireties.

### General Description

Amino acid metabolism enzymes are involved in ammonia production through degradation of amino acids. The enzymes involved include cystathionase (CTH), serine dehydratase (SDS), ornithine aminotransferase (OAT), and glutaminase 2 (GLS-2). The resulting ammonia from amino acid metabolism, as well as ammonia absorbed from the gut or produced by the skeletal muscles during exercise, or produced by ammoniagenesis by the kidney, is converted to urea by the urea cycle in hepatocytes. The urea cycle is the principal mechanism for the clearance of ammonia resulting from protein turnover, as well as for the metabolism of other nitrogenous metabolic compounds such as adenosine monophosphate, by converting ammonia to urea. The urea is then excreted through urine. Glutamine synthetase activity is also required to reduce plasma ammonia levels. A glucagon signaling pathway inhibitor such as a GCGR antibody targets the amino acid transporter to reduce amino acid uptake to hepatocytes.

Urea cycle disorders result from a genetic mutation in a gene encoding one of the enzymes in the urea cycle, enzymes responsible for converting ammonia to urea. Disruption of the urea cycle, typically through dysfunction of a urea cycle enzyme or a transporter defect, or through accumulation of metabolites or by substrate deficiencies, results in accumulation of nitrogen in the form of ammonia, a condition referred to as hyperammonemia. Elevated ammonia in the blood causes irreversible brain damage, coma, and death. Seizures are common in acute hyperammonemia and may result from cerebral damage. Subclinical seizures are common in acute hyperammonemic episodes, especially in neonates, and may be seen during the rise of glutamine even before ammonia levels are maximal. Ammonia can cause brain damage through a variety of mechanisms, a major component of which is cerebral edema through increased glutamine, though the specific roles of ammonia, glutamate, and glutamine in cerebral edema have yet to be elucidated. In addition, adverse effects on the nitric oxide production system may also contribute to injury, as may arginine deficiency due to ASL deficiency.

In addition to increases in plasma ammonia levels, changes in plasma and/or urine levels of the following may be seen in one or more forms of urea cycle disorders:

Increase or decrease in plasma citrullin;
Increase in plasma and/or urine levels of argininosuccinic acid (ASA);
Increase or decrease in plasma arginine;
Increase in plasma ornithine;
Increase in plasma glutamine;
Increase in plasma alanine;
Increase in plasma asparagine;
Increase in urinary orotic acid; or
Increase in urine homocitrulline.

In some subjects, the urea cycle disorder is associated with the presence of an

inborn error of urea synthesis, or urea cycle enzyme defect: Carbamyl Phosphate Synthetase (CPS1), N-Acetylglutamate Synthetase (NAGS), Ornithine Transcarbamylase (OTC Deficiency), Argininosuccinic Acid Synthetase (ASS, Citrullinemia), Argininosuccinate Lyase (AL or ASA Lyase, Argininosuccinic Aciduria), and Arginase (AR1).

Other inborn errors of metabolism may also cause hyperammonemia: Ornithine aminotransferase (OAT) deficiency (in neonates), Tyrosinemia type 1, Galactosemia, mitochondrial disorders, citrin deficiency leading to Citrullinemia type II (CTLN2), and neonatal intrahepatic cholestasis caused by citrin deficiency (NICCD). CTLN2 is a late-onset disorder, where hepatic dysfunction is absent, but the subject experiences recurrent hyperammonemia, delirium, irritability, and fatty liver infiltration. Citrin deficiency results from mutation of the gene SLC25A13. As citrin functions to transport aspartate/glutamate across the mitochondrial membrane to the cytoplasm, a deficiency in citrin results in a decrease in cytoplasmic aspartate which limits the activity of the enzyme argininosuccinic acid synthase. Dysfunction of another transporter, ornithine translocase, results in hyperornithinemia, homocitrullinuia, and hyperammonemia, as the reduction in ornithine transport into the mitochondria results in orotic aciduria and deficiency of urea synthesis.

Other congenital disorders which cause hyperammonemia include Propionic acidemia, Isolated methylmalonic acidemia, Isovaleric acidemia, Carbonic anhydrase VA deficiency, Lysinuric protein intolerance, carnitine palmitoyl transferase II deficiency, carnitine-acylcarnitine translocase, pyrroline-5-carboxylate synthetase deficiency, pyruvate carboxylase deficiency, carbonic anhydrase Va deficiency, hyperinsulinism-hyperammonemia syndrome, mitochondrial disorders, glutamine synthetase deficiency, fatty acid oxidation disorders (for example, Short-Chain Acyl-CoA Dehydrogenase Deficiency, Medium-Chain Acyl-Coenzyme A Dehydrogenase Deficiency, Multiple Acyl-Coenzyme A Dehydrogenase Deficiency, Very Long-Chain Acyl-Coenzyme A Dehydrogenase Deficiency), and Hyperinsulinism-hyperammonemia syndrome (Familial Hyperinsulinism). See Häberle, Archives of Biochemistry and Biophysics, 536:101-108, 2013.

Depending on the degree of the enzyme or transporter dysfunction, the onset and severity of urea cycle disorders are highly variable. Severe deficiency or total absence of activity of any of the first four enzymes in the pathway (CPS1, OTC, ASS1, and ASL) or the cofactor producer (NAGS) results in the accumulation of ammonia and other precursor metabolites during the first few days of life. Newborns with severe urea cycle dysfunction become catastrophically ill within days of birth. Symptoms include irritability, poor feeding, vomiting, and lethargy, followed by seizures, hypotonia, respiratory distress, and coma.

Those with partial urea cycle dysfunction, including children and adults, present with subtle symptoms, and often diagnosis is difficult given the symptoms are not commonly recognized. Children with mild or moderate urea cycle dysfunction may have early symptoms including failure to thrive, inconsolable crying, agitation or hyperactive behavior, and aversion to foods containing high protein. Later symptoms include frequent vomiting, lethargy, delirium, and if untreated, hyperammonemic coma and death.

Late-onset in adults is identified when a hyperammonemia crisis is triggered by a metabolic stressor including viral infection, excessive exercise or dieting, post-partum stress, parenteral nutrition with high protein administration, gastrointestinal bleeding, administration of valproic acid, administration of prednisone or other corticosteroid, infection, and post-operative stress. Symptoms typically involve episodes of disorientation, confusion, slurred speech, combativeness or agitation, stroke-like symptoms, lethargy and delirium. Subjects are often treated for psychiatric symptoms prior to diagnosis. Without treatment, a subject experiencing a hyperammonemia crisis is at risk for permanent brain damage, coma, and death.

Exemplary late-onset subjects can be a bodybuilders on high protein diets. Such subjects are high risk, having an undiagnosed urea cycle disorder that when combined with a high protein diet, triggers a hyperammonemia crisis. Such subjects can be improperly diagnosed as they appear to be otherwise very healthy individuals. The delay in diagnosis results in coma and death.

Additional causes of hyperammonemia, i.e. acquired hyperammonemia, include liver disease and complications thereof, e.g., hepatic encephalopathy in subjects with advanced liver disease, fetor hepaticus (a late sign of liver failure), vascular bypass of the liver, and biliary atresia; administration of toxic levels of valproic acid (metabolites of valproic acid inhibit NAGS, also deplete carnitine), corticosteroid, or cyclophosphamide; herpes simplex infection; and gastrointestinal bacterial overgrowth. Hyperammonemia can also be caused by infection with urease-producing organisms (increased ammonia production in the intestine or urinary tract).

Furthermore, hyperammonemia can be caused by total parenteral nutrition (with relative arginine deficiency), treatment with L-asparaginase (increased ammonia production due to hydrolysis of asparagine), nutritional carnitine deficiency (impaired fatty acid oxidation leading to lack of acetyl-CoA), cystoscopy with glycine-containing solutions (increased ammonia production from nitrogen overload), post-lung/bone marrow transplantation (reduced glutamine synthetase activity), vascular malformations, or transient hyperammonemia of the newborn. See Häberle, Archives of Biochemistry and Biophysics, 536:101-108, 2013.

Treatment options initially include hemodialysis or continuous renal replacement therapy (RRT) as soon as it's clear that the subject is experiencing a hyperammonemia crisis. Hemodialysis or RTT may be discontinued when plasma ammonia levels fall below 80 μmol/L, or below 120 μmol/L. In addition, oral or parenteral protein administration is discontinued, while administration of calories from glucose and fat helps prevent excessive catabolic state. Once a diagnosis of a UCD is made, treatment of acute manifestations can be started and treatment is tailored to the specific urea cycle disorder. Therapeutics such as intravenous or oral administration of sodium phenylacetate, sodium benzoate, sodium phenylbutyrate, or glycerol phenylbutyrate scavenge ammonia and divert nitrogen from the urea cycle to excretion through the kidneys. Sodium phenylacetate combines with glutamine, producing phenylacetylglutamine, which is excreted by the kidneys. Sodium benzoate conjugates with glycine, producing sodium hippurate, which is also excreted by the kidneys. Unfortunately, for subjects with recurrent hyperammonemia or those resistant to conventional treatment, liver transplantation is the best treatment option.

While not wishing to be held by theory, it has been determined that an antagonist of GCGR decreases the expression of amino acid metabolism enzymes in the liver of mice and monkeys. As such, blocking the glucagon signaling pathway provides a method for treating hyperammonemia and urea cycle disorders by reducing the amount of ammonia entering the urea cycle.

To date, there have been no studies examining the effects of antagonizing the glucagon signaling pathway on urea cycle disorders or conditions or diseases associated with hyperammonemia. The studies described in the Examples use an antagonist of GCGR as an exemplary inhibitor of the glucagon signaling pathway in a mouse model of urea cycle disorders to demonstrate the effects on hyperammonemia and death over several weeks of treatment.

### Definitions

The “glucagon receptor”, also referred to herein as “GCGR”, belongs to the G protein-coupled receptor class 2 family and consists of a long amino terminal extracellular domain, seven transmembrane segments, and an intracellular C-terminal domain. Glucagon receptors are notably expressed on the surface of hepatocytes where they bind to glucagon and transduce the signal provided thereby into the cell. Accordingly, the term “glucagon receptor” also refers to one or more receptors that interact specifically with glucagon to result in a biological signal. DNA sequences encoding glucagon receptors of rat and human origin have been isolated and disclosed in the art (EP0658200B1). The murine and cynomolgus monkey homologues have also been isolated and sequenced (Burcelin, et al., (1995) Gene 164:305-310); McNally et al., (2004) Peptides 25:1171-1178). As used herein, “glucagon receptor” and “GCGR” are used interchangeably. The expressions “GCGR”, “hGCGR” or fragments thereof, as used herein, refer to the human GCGR protein or fragment thereof, unless specified as being from a non-human species, e.g. “mouse GCGR”, “rat GCGR”, or “monkey GCGR”.

The phrase “GCGR antagonist” refers to an inhibitor, antagonist, or inverse agonist of the GCGR signaling pathway. A “GCG inhibitor” may prevent the binding of glucagon to the receptor. A GCGR inhibitor may also prevent the binding of glucagon to the receptor. However, both effectively block or attenuate activation of the receptor, or may interfere with the signaling cascade downstream of the GCGR activation, and are collectively referred to as “glucagon signaling pathway antagonists”.

The terms “inhibitor” or “antagonist” include a substance that retards or prevents a chemical or physiological reaction or response, for example, a glucagon signaling pathway antagonist.

A GCGR antagonist is able to bind to the glucagon receptor and thereby antagonize the activity of GCG mediated by the GCGR. Inhibiting the activity of GCG by antagonizing the binding and activity of GCG at the GCGR reduces expression of enzymes involved in amino acid metabolism. Methods by which to determine the binding of a supposed antagonist with the glucagon receptor are known in the art and means by which to determine the interference with glucagon activity at the glucagon receptor are publicly available; see, e.g., S. E. de Laszlo et al., (1999) Bioorg. Med. Chem. Lett. 9:641-646.

Contemplated as useful herein are GCGR antagonists or GCG inhibitors having as a functional component thereof a small molecule compound, or in other words a low molecular weight organic compound. A small molecule is typically less than 800 Daltons. Additionally, CRISPR technology can be used to knock-down GCG or GCGR expression. As such, in some embodiments, a glucagon signaling pathway antagonist can be selected from a small molecule inhibitor, shRNA, siRNA, peptide inhibitor, CRISPR technology (Clustered regularly interspaced short palindromic repeats; CRISPR technology can generate GCGR knock-down or deletion of regulatory sequences affecting GCGR activity), an antisense inhibitor, DARPin, Spiegelmers, aptamers, engineered Fn type-III domains, GCG or GCGR neutralizing monoclonal antibodies, and their derivatives.

An example of a glucagon signaling pathway antagonist includes, but is not limited to, an antibody (human or humanized), or an antigen binding portion thereof, to GCG or GCGR, that blocks binding or inhibits the activity of the GCGR signaling pathway. Exemplary GCGR antagonists that may be used in the methods described herein include isolated human monoclonal antibody or antigen-binding fragment thereof comprising: (a) a HCVR having an amino acid sequence selected from the group consisting of SEQ ID NO: 2, 18, 34, 50, 66, 70, 86, 90, 106, 110, 126, 130 and 146; and/or (b) a LCVR having an amino acid sequence selected from the group consisting of SEQ ID NO: 10, 26, 42, 58, 68, 78, 88, 98, 108, 118, 128, 138 and 148. Exemplary GCG inhibitors that may be used in the methods described herein include isolated human monoclonal antibody or antigen-binding fragment thereof comprising: (a) a HCVR having an amino acid sequence selected from the group consisting of SEQ ID NO: 150, 166, 182, 198, 214, 230, 246, 262, 278, and 294; and/or (b) a LCVR having an amino acid sequence selected from the group consisting of SEQ ID NO: 158, 174, 190, 206, 222, 238, 254, 270, 286, and 302.

A “therapeutically effective dose” is a dose that produces the desired effect for which it is administered. The exact dose will depend on the purpose of the treatment, and will be ascertainable by one skilled in the art using known techniques (see, for example, Lloyd (1999) The Art, Science and Technology of Pharmaceutical Compounding).

By the phrase “substantially identical” is meant a protein sequence having at least 95% identity to an HCVR having an amino acid sequence selected from the group consisting of SEQ ID NO: 2, 18, 34, 50, 66, 70, 86, 90, 106, 110, 126, 130 and 146; and/or (b) a LCVR having an amino acid sequence selected from the group consisting of SEQ ID NO: 10, 26, 42, 58, 68, 78, 88, 98, 108, 118, 128, 138 and 148, and capable of binding GCGR and inhibiting the biological activity of GCGR. The phrase “substantially identical” is also meant a protein sequence having at least 95% identify to an HCVR having an amino acid sequence selected from the group consisting of the amino acid sequences SEQ ID NO: 150, 166, 182, 198, 214, 230, 246, 262, 278, and 294; and/or (b) a LCVR having an amino acid sequence selected from the group consisting of SEQ ID NO: 158, 174, 190, 206, 222, 238, 254, 270, 286, and 302, and capable of binding GCG and inhibiting the biological activity of GCG.

The terms “identity” or “homology” are construed to mean the percentage of amino acid residues in the candidate sequence that are identical with the residue of a corresponding sequence to which it is compared, after aligning the sequences and introducing gaps, if necessary to achieve the maximum percent identity for the entire sequence, and not considering any conservative substitutions as part of the sequence identity. Neither N- or C-terminal extensions nor insertions will be construed as reducing identity or homology. Methods and computer programs for the alignment are well known in the art. Sequence identity may be measured using sequence analysis software (e.g., Sequence Analysis Software Package, Genetics Computer Group, University of Wisconsin Biotechnology Center, 1710 University Ave., Madison, Wis. 53705). This software matches similar sequences by assigning degrees of homology to various substitutions, deletions, and other modifications.

The term “treating” (or “treat” or “treatment”) refers to processes involving a slowing, interrupting, inhibiting, arresting, controlling, stopping, reducing, ameliorating, or reversing the progression, duration, or severity of an existing symptom, disorder, condition, or disease, but does not necessarily involve a total elimination of all disease-related symptoms, conditions, or disorders through use of a GCG inhibitor or GCGR antagonist as described herein. Furthermore, “treating”, “treatment” or “treat” refers to an approach for obtaining beneficial or desired results including clinical results, which include, but are not limited to, one or more of the following: inhibiting, delaying or preventing the progression of hyperammonemia and/or a urea cycle disorder; inhibiting, delaying or preventing the progression of a disease associated with hyperammonemia, or characterized by elevated plasma ammonia levels such as in chronic liver disease or toxic administration of valproic acid, or a condition or disease associated with the presence of a gene variant reported to cause a urea cycle disorder; or inhibiting, preventing, or ameliorating at least one symptom associated with a disease associated with hyperammonemia; or lowering blood ammonia levels, such that the condition or disease associated with hyperammonemia is mediated, or at least one symptom or complication associated with the condition or disease is alleviated or reduced in severity. “Treatment” or “treating”, as used herein, also refers to increasing the quality of life of those suffering from the disease, decreasing the dose of other medications required to treat the disease and/or prolonging survival of subjects. For example, “treatment” or “treating” can include reducing the amount and/or dosage of sodium phenylbutyrate or sodium benzoate necessary to treat a subject with hyperammonemia.

The term “hyperammonemia” is a state in which plasma ammonia levels are greater than normal, e.g. above 30 μmol/L, or above 50 μmol/L, or above 80 μmol/L, or above 100 μmol/L, or above 120 μmol/L. Plasma ammonia levels in severe hyperammonemia are above 1000 μmol/L.

### Glucagon Signaling Pathway Inhibitors

Provided herein are glucagon signaling pathway antagonists, e.g., GCG inhibitors and GCGR antagonists, for the treatment of conditions or diseases characterized by hyperammonemia. In some embodiments, the antagonist is an inhibitor of glucagon, for example, amylin and pramlintide. In some embodiments, the antagonist is an inhibitor of GCGR. In some embodiments, the GCGR antagonist is MK-0893, PF-06291874, LGD-6972, or LY2409021.

In some embodiments, the antagonist comprises an antibody capable of binding GCG or GCGR, or a fragment thereof. In some embodiments, the signaling pathway is inhibited by the interruption of GCG or GCGR expression, by, for example, using CRISPR technology or antisense, or by targeting a downstream enzyme such as CaMKII.

In some embodiments, the GCG inhibitor or GCGR antagonist is an antisense molecule (GR-ASO), antibody, small molecule inhibitor, shRNA, siRNA, peptide inhibitor (amylin, pramlintide), DARPin, Spiegelmer, aptamer, engineered Fn type-III domains, or a derivative thereof.

### Anti-GCGR Antibodies, Anti-GCG Antibodies, and Antibody Fragments

In some embodiments, the GCGR antagonist is an antibody or antibody fragment as disclosed in U.S. Pat. No. 8,545,847, incorporated by reference herein in its entirety. Antibodies disclosed therein are provided in Table 1.

TABLE 1

| | SEQ ID NOs: | | | | | | | |
|---|---|---|---|---|---|---|---|---|
| Antibody Designation | HCVR | HCDR1 | HCDR2 | HCDR3 | LCVR | LCDR1 | LCDR2 | LCDR3 |
| H4H1345N | 2 | 4 | 6 | 8 | 10 | 12 | 14 | 16 |
| H4H1617N | 18 | 20 | 22 | 24 | 26 | 28 | 30 | 32 |
| H4H1765N | 34 | 36 | 38 | 40 | 42 | 44 | 46 | 48 |
| H4H1321B | 50 | 52 | 54 | 56 | 58 | 60 | 62 | 64 |
| H4H1321P | 66 | 52 | 54 | 56 | 68 | 60 | 62 | 64 |
| H4H1327B | 70 | 72 | 74 | 76 | 78 | 80 | 82 | 84 |
| H4H1327P | 86 | 72 | 74 | 76 | 88 | 80 | 82 | 84 |
| H4H1328B | 90 | 92 | 94 | 96 | 98 | 100 | 102 | 104 |
| H4H1328P | 106 | 92 | 94 | 96 | 108 | 100 | 102 | 104 |
| H4H1331B | 110 | 112 | 114 | 116 | 118 | 120 | 122 | 124 |
| H4H1331P | 126 | 112 | 114 | 116 | 128 | 120 | 122 | 124 |
| H4H1339B | 130 | 132 | 134 | 136 | 138 | 140 | 142 | 144 |
| H4H1339P | 146 | 132 | 134 | 136 | 148 | 140 | 142 | 144 |

Additional GCGR antibodies or antibody fragments contemplated as useful herein include those disclosed in U.S. Pat. Nos. 5,770,445 and 7,947,809; European patent application EP2074149A2; EP patent EP0658200B1; U.S. patent publications 2009/0041784;2009/0252727; and 2011/0223160; and PCT publication WO2008/036341. The patents and publications are incorporated by reference herein in their entirety.

In some embodiments, the GCG inhibitor is an antibody or antibody fragment thereof as disclosed in U.S. 2016/0075778, incorporated by reference herein in its entirety. Antibodies disclosed therein are provided in Table 2.

TABLE 2

| | SEQ ID NOs: | | | | | | | |
|---|---|---|---|---|---|---|---|---|
| Antibody Designation | HCVR | HCDR1 | HCDR2 | HCDR3 | LCVR | LCDR1 | LCDR2 | LCDR3 |
| H1H059P | 150 | 152 | 154 | 156 | 158 | 160 | 162 | 164 |
| H4H10223P | 166 | 168 | 170 | 172 | 174 | 176 | 178 | 180 |
| H4H10231P | 182 | 184 | 186 | 188 | 190 | 192 | 194 | 196 |
| H4H10232P | 198 | 200 | 202 | 204 | 206 | 208 | 210 | 212 |
| H4H10236P | 214 | 216 | 218 | 220 | 222 | 224 | 226 | 228 |
| H4H10237P | 230 | 232 | 234 | 236 | 238 | 240 | 242 | 244 |
| H4H10238P | 246 | 248 | 250 | 252 | 254 | 256 | 258 | 260 |
| H4H10250P | 262 | 264 | 266 | 268 | 270 | 272 | 274 | 276 |
| H4H10256P | 278 | 280 | 282 | 284 | 286 | 288 | 290 | 292 |
| H4H10270P | 294 | 296 | 298 | 300 | 302 | 304 | 306 | 308 |

Additional GCG antibodies or antibody fragments contemplated as useful herein include those disclosed in U.S. Pat. Nos. 4,206,199; 4,221,777; 4,423,034; 4,272,433; 4,407,965; 5,712,105; and PCT publications WO2007/124463 and WO2013/081993.

Antibody fragments include any fragment having the required target specificity, e.g. antibody fragments either produced by the modification of whole antibodies (e.g. enzymatic digestion), or those synthesized de novo using recombinant DNA methodologies (scFv, single domain antibodies, DVD (dual variable domain immunoglobulins), or dAbs (single variable domain antibodies)) or those identified using human phage or yeast display libraries (see, for example, McCafferty et al. (1990) Nature 348:552-554). Alternatively, antibodies can be isolated from mice producing human, human-mouse, human-rat, and human-rabbit chimeric antibodies using standard immunization and antibody isolation methods, including but not limited to making hybridomas, or using B cell screening technologies, such as SLAM. Immunoglobulin binding domains also include, but are not limited to, the variable regions of the heavy (V_{H}) or the light (V_{L}) chains of immunoglobulins. Or by immunizing people and isolating antigen positive B cells and cloning the cDNAs encoding the heavy and light chain and coexpressing them in a cell, such as CHO.

The term “antibody” as used herein refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant regions, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Within each IgG class, there are different isotypes (eg. IgG1, IgG2, IgG3, IgG4). Typically, the antigen-binding region of an antibody will be the most critical in determining specificity and affinity of binding.

An exemplary immunoglobulin (antibody) structural unit comprises a tetramer. Each tetramer is composed of two identical pairs of polypeptide chains, each pair having one light chain (about 25 kD) and one heavy chain (about 50-70 kD). The N-terminus of each chain defines a variable region of about 100-110 or more amino acids primarily responsible for antigen recognition. The terms “variable light chain” (V_{L}) and variable heavy chain (V_{H}) refer to these light and heavy chains respectively.

Antibodies exist as intact immunoglobulins, or as a number of well-characterized fragments produced by digestion with various peptidases. For example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. While various antibody fragments are defined in terms of the digestion of an intact antibody, one of skill will appreciate that such fragments may be synthesized de novo either chemically or by using recombinant DNA methodology.

Methods for preparing antibodies useful according to the methods herein are known to the art. See, for example, Kohler & Milstein (1975) Nature 256:495-497; Harlow & Lane (1988) Antibodies: a Laboratory Manual, Cold Spring Harbor Lab., Cold Spring Harbor, N.Y.). The genes encoding the heavy and light chains of an antibody of interest can be cloned from a cell, e.g., the genes encoding a monoclonal antibody can be cloned from a hybridoma and used to produce a recombinant monoclonal antibody. Monoclonal antibodies can be humanized using standard cloning of the CDR regions into a human scaffold. Gene libraries encoding human heavy and light chains of monoclonal antibodies can also be made from hybridoma or plasma cells. Random combinations of the heavy and light chain gene products generate a large pool of antibodies with different antigenic specificity. Techniques for the production of single chain antibodies or recombinant antibodies (U.S. Pat. Nos. 4,946,778; 4,816,567) can be adapted to produce antibodies used in the methods disclosed herein. Also, transgenic mice, or other organisms such as other mammals, may be used to express human, human-mouse chimeric, human-rat chimeric, human-rabbit chimeric, or humanized antibodies. Alternatively, phage display or yeast display technology can be used to identify human antibodies and heteromeric Fab fragments that specifically bind to selected antigens.

### Immunoconjugates

The disclosure encompasses treatment of hyperammonemia with a human anti-GCGR monoclonal antibody (or human anti-GCG monoclonal antibody) conjugated to a therapeutic moiety (“immunoconjugate”), such as an agent that is capable of reducing blood ammonia levels or addressing another symptom of hyperammonemia. The type of therapeutic moiety that may be conjugated to the anti-GCGR antibody will take into account the condition to be treated and the desired therapeutic effect to be achieved. For example, in an effort to lower blood ammonia levels, and/or to maintain normal blood ammonia levels, an agent such as a glucagon receptor pathway antagonist, or a second GCGR inhibitor or GCG inhibitor may be conjugated to the GCGR antibody. Alternatively, if the desired therapeutic effect is to reduce glutamine or any other symptoms or conditions associated with a urea cycle disorder, it may be advantageous to conjugate an appropriate agent to the anti-GCGR antibody. Examples of suitable agents for forming immunoconjugates are known in the art.

### Multi-Specific Antibodies

The antibodies useful according to the methods provided herein may be mono-specific, bi-specific, or multi-specific. Multi-specific antibodies may be specific for different epitopes of one target polypeptide or may contain antigen-binding domains specific for more than one target polypeptide. See, e.g., Tutt et al., (1991) J. Immunol. 147:60-69; Kufer et al., (2004) Trends Biotechnol. 22:238-244. The anti-GCGR antibodies can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bi-specific or a multi-specific antibody with a second binding specificity. For example, bi-specific antibodies are contemplated where one arm of an immunoglobulin is specific for human GCGR or a fragment thereof, and the other arm of the immunoglobulin is specific for a second therapeutic target or is conjugated to a therapeutic moiety. In certain embodiments, one arm of an immunoglobulin is specific for an epitope on the N-terminal domain of hGCGR or a fragment thereof, and the other arm of the immunoglobulin is specific for an epitope on one of the EC loops of hGCGR, or a fragment thereof. In certain embodiments, one arm of an immunoglobulin is specific for one EC loop, or a fragment thereof, and the second arm is specific for a second EC loop, or a fragment thereof. In certain embodiments, one arm of an immunoglobulin is specific for one epitope on one EC loop of hGCGR and the other arm is specific for a second epitope on the same EC loop of hGCGR.

An exemplary bi-specific antibody format that can be used according to the methods described herein involves the use of a first immunoglobulin (Ig) C_{H}3 domain and a second Ig C_{H}3 domain, wherein the first and second Ig C_{H}3 domains differ from one another by at least one amino acid, and wherein at least one amino acid difference reduces binding of the bi-specific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L18M, N44S, K52N, V57M, and V82I (by IMGT; D356E, L358M, N384S, K392N, V397M, and V422I by EU) in the case of IgG1antibodies; N44S, K52N, and V821 (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies. Variations on the bi-specific antibody format described above are contemplated within the scope of the present disclosure.

### Antibody Screening and Selection

Screening and selection of preferred antibodies, useful according to the methods provided herein, can be conducted by a variety of methods known to the art. Initial screening for the presence of monoclonal antibodies specific to a target antigen may be conducted through the use of ELISA-based methods, for example. A secondary screen is preferably conducted to identify and select a desired monoclonal antibody for use in construction of antibody-drug conjugates. Secondary screening may be conducted with any suitable method known to the art. One preferred method, termed “Biosensor Modification-Assisted Profiling” (“BiaMAP”) is described in U.S. Publication 2004/0101920, herein specifically incorporated by reference in its entirety. BiaMAP allows rapid identification of hybridoma clones producing monoclonal antibodies with desired characteristics. More specifically, monoclonal antibodies are sorted into distinct epitope-related groups based on evaluation of antibody: antigen interactions. Antibodies capable of blocking either a ligand or a receptor may be identified by a cell based assay, such as a luciferase assay utilizing a luciferase gene under the control of an NFκB driven promoter or CAMP response driven promoter. Stimulation of the GCGR by glucagon leads to a signal through NFκB/cAMP/CREB thus increasing luciferase levels in the cell. Blocking antibodies are identified as those antibodies that blocked glucagon induction of luciferase activity.

### Treatment Population

The therapeutic methods provided herein are useful for treating individuals with a urea cycle disorder or a condition or disease associated with hyperammonemia. In some embodiments, the subject suffers from congenital hyperammonemia, e.g. a defect in a urea cycle enzyme selected from the group consisting of carbamyl phosphate synthetase (CPS1), N-acetylglutamate synthetase (NAGS), ornithine transcarbamylase (OTC), argininosuccinic acid synthetase (ASS), argininosuccinate lyase (ASL), and arginase (AR1); or a defect in a urea cycle transporter selected from ornithine translocase (ORNT1) and citrin. Other congenital disorders which cause hyperammonemia include Propionic acidemia, Isolated methylmalonic acidemia, Isovaleric acidemia, Carbonic anhydrase VA deficiency, Lysinuric protein intolerance, carnitine palmitoyl transferase II deficiency, carnitine-acylcarnitine translocase, pyrroline-5-carboxylate synthetase deficiency, pyruvate carboxylase deficiency, ornithine aminotransferase deficiency, carbonic anhydrase Va deficiency, hyperinsulinism-hyperammonemia syndrome, mitochondrial disorders, glutamine synthetase deficiency, fatty acid oxidation disorders (for example, Short-Chain Acyl-CoA Dehydrogenase Deficiency, Medium-Chain Acyl-Coenzyme A Dehydrogenase Deficiency, Multiple Acyl-Coenzyme A Dehydrogenase Deficiency, Very Long-Chain Acyl-Coenzyme A Dehydrogenase Deficiency), and Hyperinsulinism-hyperammonemia syndrome (Familial Hyperinsulinism).

In some embodiments, the hyperammonemia is acquired, e.g. is caused by liver disease and complications thereof; is caused by treatment with a therapeutic agent (L-asparaginase or pegaspargase), 5-pentanoic acid, valproic acid, a corticosteroid, or a cyclophosphamide; or is caused by herpes simplex infection or hepatitis B infection. Additional causes of hyperammonemia, i.e. acquired hyperammonemia, include liver disease and complications thereof, e.g., hepatic encephalopathy in subjects with advanced liver disease, fetor hepaticus (a late sign of liver failure), vascular bypass of the liver, and biliary atresia; administration of toxic levels of valproic acid, corticosteroid, or cyclophosphamide; herpes simplex infection; and gastrointestinal bacterial overgrowth. Hyperammonemia can also be caused by infection with urease-producing organisms.

Furthermore, hyperammonemia can be caused by total parenteral nutrition (with relative arginine deficiency), nutritional carnitine deficiency, cystoscopy with glycine-containing solutions, post-lung/bone marrow transplantation, vascular malformations, or transient hyperammonemia of the newborn.

In some embodiments, elevated levels of ammonia is detected in the subject sera. In some embodiments, excess glutamine is detected in the subject sera.

### Therapeutic Administration and Formulations

Useful according to the methods provided herein are therapeutic compositions comprising a glucagon/GCGR antagonist, such as, for example, an anti-GCGR antibody. The administration of therapeutic compositions in accordance with the methods described herein will be administered via a suitable route including, but not limited to, intravenously, subcutaneously, intramuscularly, intrathecally, intracerebrally, intraventricularly, intranasally, or orally, with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody may vary depending upon the age and the size of a subject to be administered, target disease, conditions, route of administration, and the like. When the antibody is used for treating hyperammonemia or lowering blood ammonia levels associated with a urea cycle disorder, in a subject, it is advantageous to intravenously administer the antibody normally at a dose of about 0.01 to about 30 mg/kg body weight, more preferably about 0.02 to about 7, about 0.03 to about 5, or about 0.05 to about 3 mg/kg body weight. Depending on the severity of the condition and response to treatment, the frequency and the duration of the treatment can be adjusted. In certain embodiments, the antibody or antigen-binding fragment thereof can be administered as an initial dose of at least about 0.1 mg to about 800 mg, about 1 to about 500 mg, about 5 to about 300 mg, or about 10 to about 200 mg, to about 100 mg, or to about 50 mg.

In certain embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks; or until the hyperammonemia is resolved.

Various delivery systems are known and can be used to administer the pharmaceutical composition comprising the antibody, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, depot formulations, aerosol, intradermal, transdermal, intramuscular, intraperitoneal, intravenous, subcutaneous, intranasal, epidural, intrathecal, intraventricular, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The pharmaceutical composition can be also delivered in a vesicle, in particular a liposome (see, for example, Langer (1990) Science 249:1527-1533).

In certain situations, the pharmaceutical composition can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

A pharmaceutical composition useful herein can be delivered subcutaneously or intravenously with a standard needle and syringe. In addition, with respect to subcutaneous delivery, a pen delivery device readily has applications in delivering a pharmaceutical composition useful in the methods described herein. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition useful according to the methods described herein. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, Inn.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition useful according to the methods described herein include, but certainly are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.) and the HUMIRA™ Pen (Abbott Labs, Abbott Park, Ill.), to name only a few.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 750 mg per dosage form in a unit dose; especially in the form of injection, it is preferred that the aforesaid antibody is contained in about 5 to about 100 mg and in about 10 to about 250 mg for the other dosage forms.

### Combination Therapies

In numerous embodiments, the GCG inhibitors or GCGR antagonists useful herein may be administered in combination with one or more additional compounds, therapeutic agents, or therapies. Combination therapy may be simultaneous (or concomitant) or sequential. In some aspects, the additional compound (or therapeutic agent) is formulated into the same pharmaceutical composition as the GCG inhibitor or GCGR antagonist. In some aspects, the additional compound is administered separately, before, after, or concomitantly with administration of the GCG inhibitor or the GCGR antagonist.

In some embodiments, the glucagon signaling pathway antagonist is administered with at least one additional therapeutic agent selected from the following: insulin, a non-absorbable antibiotic (rifaximin or lactulose), sodium phenylbutyrate, sodium benzoate, sodium phenylacetate, glycerol phenylbutyrate, carbamyl glutamate (Carbaglu®), a second GCG inhibitor, and a second GCGR antagonist. In some embodiments, the glucagon signaling pathway antagonist is administered with hemodialysis or continuous renal replacement. In some embodiments, the glucagon signaling pathway antagonist is administered with L-citrulline or L-arginine free base. In some embodiments, the glucagon signaling pathway antagonist is administered with antioxidants or electrolytes. In some embodiments, the glucagon signaling pathway antagonist is administered with amino acid formulas selected from Cyclinex, EAA, UCD I&II, and individual branched chain amino acids.

The additional therapeutically active component(s) may be administered prior to, concurrent with, or after the administration of the glucagon signaling pathway antagonist, e.g. the GCG inhibitor or the GCGR antagonist. For purposes of the present disclosure, such administration regimens are considered the administration of a glucagon signaling pathway antagonist “in combination with” a second therapeutically active component.

### Administration Regimens

According to certain embodiments described herein, multiple doses of the glucagon/GCGR signaling pathway antagonist may be administered to a subject over a defined time course. The methods comprise sequentially administering to a subject multiple doses of a glucagon/GCGR signaling pathway antagonist. As used herein, “sequentially administering” means that each dose of the antagonist is administered to the subject at a different point in time, e.g., on different days separated by a predetermined interval (e.g., hours, days, weeks or months). The methods described herein comprise sequentially administering to the subject a single initial dose of the glucagon/GCGR signaling pathway antagonist, followed by one or more secondary doses of the glucagon/GCGR signaling pathway antagonist, and optionally followed by one or more tertiary doses of the glucagon/GCGR signaling pathway antagonist.

The terms “initial dose,” “secondary doses,” and “tertiary doses,” refer to the temporal sequence of administration of an glucagon/GCGR signaling pathway antagonist useful herein. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the glucagon/GCGR signaling pathway antagonist, but generally may differ from one another in terms of frequency of administration. In certain embodiments, however, the amount of the glucagon/GCGR signaling pathway antagonists contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

### Pharmaceutical Compositions

The methods disclosed herein contemplate the use of pharmaceutical compositions comprising at least a therapeutically effective amount of an active agent useful in treating hyperammonemia or a urea cycle disorder, such as a glucagon signaling pathway antagonist, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In one embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents useful according to the methods described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the active agent which will be effective in the treatment of hyperammonemia can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20 micrograms to 2 grams of active compound per kilogram body weight. Suitable dosage ranges for intra-nasal administration are generally about 0.01 pg/kg body weight to 1 mg/kg body weight. Effective doses may be extrapolated from dose-response curves derived from in vitro or animal model test systems.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the compounds that are sufficient to maintain therapeutic effect. In cases of local administration or selective uptake, the effective local concentration of the compounds may not be related to plasma concentration. One having skill in the art will be able to optimize therapeutically effective local dosages without undue experimentation.

The amount of compound administered will, of course, be dependent on the subject being treated, on the subject's weight, the severity of the affliction, the manner of administration, and the judgment of the prescribing physician. The therapy may be repeated intermittently while symptoms are detectable or even when they are not detectable. The therapy may be provided alone or in combination with other drugs.

### Kits

Also provided herein is an article of manufacturing comprising packaging material and a pharmaceutical agent contained within the packaging material, wherein the pharmaceutical agent comprises at least one glucagon signaling pathway antagonist useful according to the methods disclosed herein, and wherein the packaging material comprises a label or package insert which indicates that the glucagon signaling pathway antagonist can be used for treating a urea cycle disorder or a condition or disease characterized by hyperammonemia.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

### EXAMPLES

The following examples are provided such that those of ordinary skill in the art have a complete disclosure and description of how to implement the methods disclosed herein. Efforts have been made to ensure accuracy with respect to numbers used (e.g., amounts, temperature, etc.) but some experimental errors and deviations should be accounted for. Unless indicated otherwise, parts are by weight, molecular weight is average molecular weight, temperature is in degrees Centigrade, and pressure is at or near atmospheric.

### Example 1

### Evaluation of a GCGR Antagonist (H4H1327P) in a Mouse Model of Urea Cycle Disorder

The effects of mAb1, a GCGR monoclonal antibody having an HCVR/LCVR amino acid sequence pair of SEQ ID NOs: 86/88 (H4H1327P), on body weight, survival rate, plasma ammonia levels and blood glucose levels were determined in ornithine transcarbamylase mutant (Otc^{spf-ash}) mice, a mouse model of urea cycle disorder. Otc^{spf-ash} mutation is a G to A missense transition of the last nucleotide of exon 4 in mouse Otc gene. The mutation changes an arginine to histidine (R129H) and results in inefficient mRNA splicing at this site and a site located 48 bases into the adjacent intron. The allele is hypomorphic and leads to 5-10% retention of wild-type hepatic ornithine transcarbamylase enzyme activity. The Otc mutant mice are viable, fertile, and do not show overt sickness, but are smaller than wild-type mice. It has been shown that high protein (40% as opposed to 21% in regular chow) diet in Otc mutant mice causes hyperammonemia and 30% lethality in one week (Yang et al., A dual AAV system enables the Cas9-mediated correction of a metabolic liver disease in newborn mice. Nat Biotechnol. 2016 34:334-8). In this experiment, Otc mutant mice were placed on 30% protein to induce mild hyperammonemia and 60% lethality in 9 weeks.

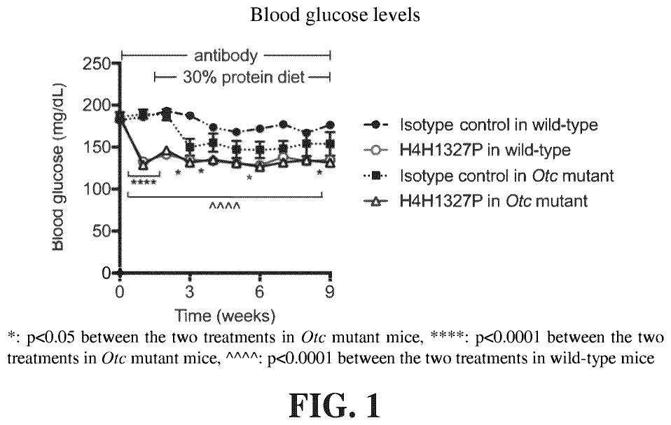

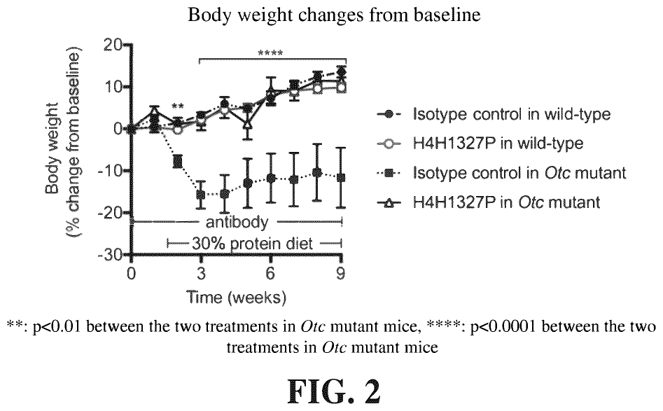

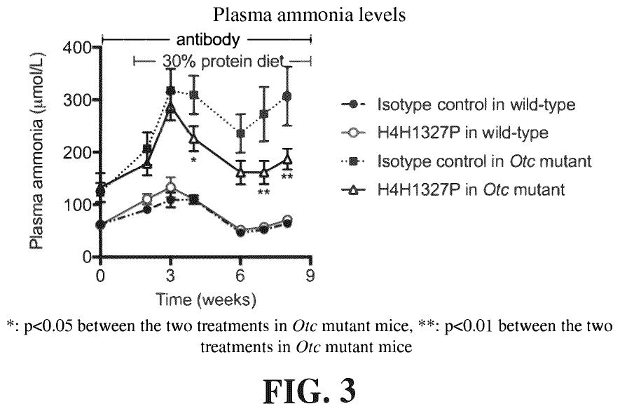

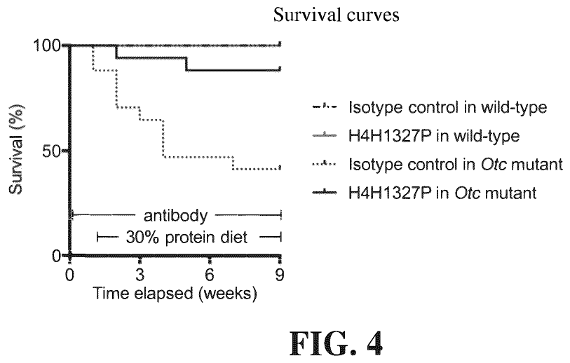

Forty-one wild-type mice and thirty-four Otc mutant mice were used in this study. To establish a baseline for blood glucose measurements, non-fasted blood glucose was tested in blood collected from the tail of each mouse using an ACCU-CHEK® Compact Plus (Roche) at 9:00 AM on day 0. The wild-type and Otc mutant were each sorted into 2 treatment groups based on their blood glucose level so that the mean glucose level across treatment groups was approximately equal. From day 0 to the end of the study, half of the wild-type (n=21) and Otc mutant (n=17) mice received weekly subcutaneous injections of mAb1 at 10 mg/kg, whereas the other half of wild-type (n=20) and Otc mutant (n=17) mice received weekly subcutaneous injections of hIgG4 isotype control at 10 mg/kg. All mice were placed on high protein diet (30% protein by weight) from day 10 for the duration of study. Non-fasting blood glucose and body weights were measured weekly. Mean±SEM of blood glucose levels at each time point was calculated for each group and shown in Table 3 and FIG. 1. Mean±SEM of body weight changes from baseline (day 0) at each time point was calculated for each group shown in Table 4 and FIG. 2. Plasma was collected at baseline and weeks 2, 3, 4, 6, 7, and 8 to determine ammonia levels. Mean±SEM of plasma ammonia levels at each time point was calculated for each group and shown in Table 5 and FIG. 3. Deaths of animals were recorded daily. Survival curve of each group is shown in FIG. 4. For blood glucose, body weight, and plasma ammonia, statistical analyses were performed by two-way ANOVA comparing each treatment group within each genotype, followed by Bonferroni post-tests.

### Results and Conclusions:

mAb1-treated wild-type mice and Otc mutant mice showed reductions in blood glucose compared to isotype control-administered animals post mAb1 administration (between weeks 1 and 9), confirming glucose lowering efficacy of mAb1 (Table 3 and FIG. 1). High protein diet induced about 15% body weight loss in isotype-control administered Otc mutant mice, whereas mAb1-administered Otc mutant mice were protected from the diet induced weight loss (Table 4 and FIG. 2). High protein diet-induced elevations in plasma ammonia levels were smaller in mAb1-treated Otc mutant mice compared to isotype-control administered Otc mutant mice (Table 5 and FIG. 3). None of the wild-type mice, regardless of the treatment, died during the study. At the end of study, 88% (15 out 17) of mAb1-treated Otc mutant mice were alive, whereas 47% (8 out of 17) of isotype-control administered Otc mutant mice were alive (FIG. 4).

These data suggest that mAb1 confers protection against hyperammonemia, excessive weight loss, and deaths in Otc mutant mice on high protein diet, in a mouse model of urea cycle disorders.

TABLE 3
Blood glucose levels

| | | Isotype | | Isotype | |
|---|---|---|---|---|---|
| | | control in | mAb1 in | control in | mAb1 in |
| | Time | wild-type | wild-type | Otc mutant | Otc mutant |
| | (weeks) | mice | mice | mice | mice |
| Blood | 0 | 182 ± 3 | 184 ± 2 | 186 ± 6 | 187 ± 4 |
| glucose | 1 | 186 ± 4 | 133 ± 2 | 189 ± 5 | 129 ± 2 |
| (mg/dL) | 2 | 193 ± 4 | 141 ± 2 | 189 ± 7 | 146 ± 2 |
| | 3 | 188 ± 3 | 135 ± 2 | 150 ± 10 | 132 ± 4 |
| | 4 | 174 ± 3 | 133 ± 3 | 155 ± 11 | 135 ± 3 |
| | 5 | 168 ± 3 | 131 ± 3 | 147 ± 10 | 131 ± 3 |
| | 6 | 172 ± 3 | 129 ± 3 | 147 ± 10 | 127 ± 4 |
| | 7 | 177 ± 4 | 138 ± 3 | 148 ± 9 | 132 ± 4 |
| | 8 | 167 ± 3 | 133 ± 3 | 154 ± 15 | 134 ± 4 |
| | 9 | 176 ± 2 | 135 ± 3 | 154 ± 14 | 132 ± 3 |

TABLE 4
Body weight changes from baseline

| | | Isotype | | Isotype | |
|---|---|---|---|---|---|
| | | control in | mAb1 in | control in | mAb1 in |
| | Time | wild-type | wild-type | Otc mutant | Otc mutant |
| | (weeks) | mice | mice | mice | mice |
| Body | 0 | 0.0 ± 0.0 | 0.0 ± 0.0 | 0.0 ± 0.0 | 0.0 ± 0.0 |
| weight (% | 1 | 0.6 ± 1.4 | 0.4 ± 0.4 | 2.5 ± 0.6 | 4.4 ± 1.1 |
| change | 2 | 1.4 ± 0.7 | −0.1 ± 0.6 | −7.6 ± 1.4 | 1.1 ± 1.6 |
| from | 3 | 3.4 ± 0.7 | 2.3 ± 0.6 | −15.6 ± 3.2 | 1.9 ± 2.1 |
| baseline) | 4 | 6.0 ± 0.7 | 4.6 ± 0.6 | −15.4 ± 4.5 | 5.1 ± 2.5 |
| | 5 | 4.8 ± 0.9 | 5.1 ± 1.0 | −12.9 ± 5.8 | 1.2 ± 3.7 |
| | 6 | 7.3 ± 1.7 | 8.1 ± 1.0 | −11.7 ± 5.8 | 9.2 ± 3.1 |
| | 7 | 10.5 ± 1.0 | 9.1 ± 1.1 | −12.1 ± 6.4 | 9.0 ± 2.2 |
| | 8 | 12.4 ± 1.1 | 9.6 ± 1.0 | −10.4 ± 6.8 | 11.5 ± 2.2 |
| | 9 | 13.5 ± 1.3 | 9.9 ± 1.1 | −11.7 ± 7.2 | 11.3 ± 2.1 |

TABLE 5
Plasma ammonia levels

| | | Isotype | | Isotype | |
|---|---|---|---|---|---|
| | | control in | mAb1 in | control in | mAb1 in |
| | Time | wild-type | wild-type | Otc mutant | Otc mutant |
| | (weeks) | mice | mice | mice | mice |
| Plasma | 0 | 63 ± 5 | 62 ± 5 | 123 ± 18 | 132 ± 28 |
| ammonia | 2 | 90 ± 8 | 110 ± 10 | 207 ± 31 | 178 ± 23 |
| (μmol/L) | 3 | 109 ± 14 | 133 ± 19 | 317 ± 41 | 288 ± 27 |
| | 4 | 110 ± 8 | 111 ± 7 | 310 ± 36 | 226 ± 24 |
| | 6 | 47 ± 2 | 52 ± 4 | 236 ± 37 | 161 ± 23 |
| | 7 | 52 ± 3 | 57 ± 3 | 273 ± 52 | 161 ± 22 |
| | 8 | 64 ± 3 | 71 ± 3 | 308 ± 56 | 187 ± 20 |

A second, differently designed study was performed, as follows, to determine the effect of H4H1327P on mice with a urea cycle disorder.

The effects of H4H1327P on plasma ammonia levels, survival rate, body weight, and blood glucose levels were determined in ornitine transcarbamylase mutant (Otc^{spf-ash}) mice, a mouse model of urea cycle disorder. Ornitine transcarbamylase mutant (Otc^{spf-ash}) mice were placed on high protein diet (30% as opposed to 21% in regular chow) first. Two days post the diet initiation, development of mild hyperammonemia was confirmed in the Otc mutant mice, before antibody administrations initiated.

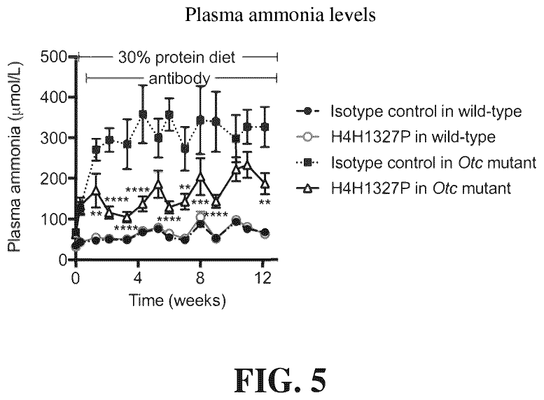

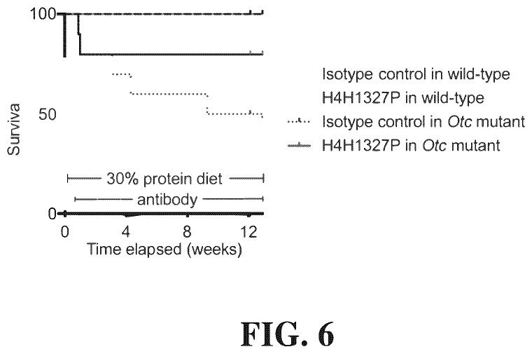

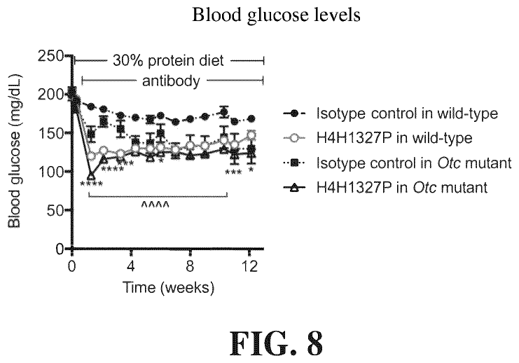

Twenty wild-type mice and twenty Otc mutant mice were used in this study. To establish a baseline for plasma ammonia measurements, non-fasted ammonia level was tested in plasma collected from the submandibular gland of each mouse using ADVIA® 1800 blood chemistry analyzer (Bayer, Leverkusen, Germany) at 9:00 AM on day 0. All mice were placed on high protein diet (30% protein by weight) from day 0 for the duration of study. The wild-type and Otc mutants were each sorted into 2 treatment groups based on their plasma ammonia level on day 2 so that the mean ammonia level of each treatment group in each genotype was approximately equal. From day 2 to the end of the study, a half of wild-type (n=10) and Otc mutant (n=10) mice received weekly subcutaneous injections of H4H1327P at 10 mg/kg, whereas the other half of wild-type (n=10) and Otc mutant (n=10) mice received weekly subcutaneous injections of hIgG4 isotype control at 10 mg/kg. Plasma was collected at baseline, day 2 and thereafter every 5 to 12 days to determine ammonia levels. Mean±SEM of plasma ammonia levels at each time point was calculated for each group and shown in Table 6 and FIG. 5. Deaths of animals were recorded daily. Survival curves of each group are shown in FIG. 6. Body weights and non-fasting blood glucose were measured weekly. Mean±SEM of body weight changes from baseline (day 0) at each time point was calculated for each group and shown in Table 7 and FIG. 8. Mean±SEM of blood glucose levels at each time point was calculated for each group and shown in Table 8 and FIG. 8. For blood glucose, body weight, and plasma ammonia, statistical analyses were performed by two-way ANOVA comparing each treatment groups within each genotype, followed by Bonferroni post-tests.

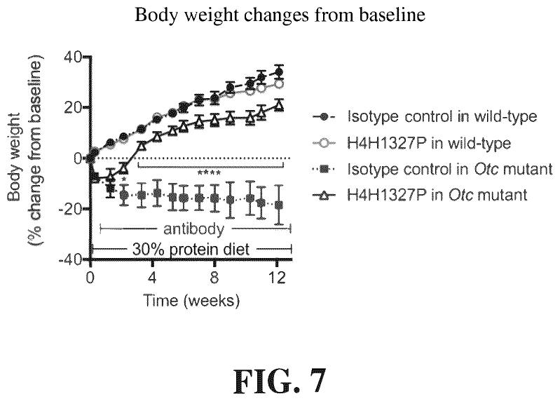

High protein diet increased plasma ammonia levels in Otc mutant mice from 65.2 to 129.4 μmol/L in two days. Otc mutant mice administered with isotype-control showed further elevated mean plasma ammonia levels of 271-359 μmol/L, whereas mean plasma ammonia levels in H4H1327P-adminisited Otc mutant mice remained within 105-232 μmol/L for the duration of the study (Table 6 and FIG. 5). At the end of the study, 80% (8 out 10) of H4H1327P-treated Otc mutant mice were alive, whereas 40% (4 out of 10) of isotype-control administered Otc mutant mice were alive (FIG. 6). None of the wild-type mice, regardless of the treatment, died during the study. High protein diet induced 18.5% body weight loss in isotype-control administered Otc mutant mice, whereas H4H1327P-administered Otc mutant mice showed 20.9% increases in body weight at the end of the study (Table 7 and FIG. 7). H4H1327P-treated wild-type mice and Otc mutant mice showed reductions in blood glucose compared to isotype control-administered animals post H4H1327P administration, confirming glucose lowering efficacy of H4H1327P (Table 8 and FIG. 8).

In summary, H4H1327P was able to ameliorate hyperammonemia, excessive weight loss, and deaths, triggered by high protein diet in Otc mutant mice. These data indicate that H4H1327P is a useful option for patients with urea cycle disorders.

TABLE 6
Plasma ammonia levels

| | | Isotype | | Isotype | |
|---|---|---|---|---|---|
| | | control in | H4H1327P | control in | H4H1327P in |
| | Time | wild-type | in wild- | Otc mutant | Otc mutant |
| | (weeks) | mice | type mice | mice | mice |
| Plasma | 0 | 35 ± 2 | 32 ± 1 | 68 ± 8 | 63 ± 7 |
| ammonia | 0.3 | 44 ± 3 | 44 ± 3 | 126 ± 16 | 133 ± 20 |
| (μmol/L) | 1.3 | 47 ± 3 | 55 ± 3 | 271 ± 27 | 170 ± 42 |
| | 2.1 | 50 ± 1 | 52 ± 3 | 295 ± 29 | 116 ± 15 |
| | 3.3 | 49 ± 2 | 50 ± 5 | 284 ± 61 | 105 ± 13 |
| | 4.3 | 68 ± 5 | 71 ± 5 | 359 ± 72 | 137 ± 18 |
| | 5.3 | 76 ± 4 | 79 ± 7 | 299 ± 48 | 186 ± 33 |
| | 6.0 | 55 ± 5 | 64 ± 9 | 358 ± 40 | 129 ± 15 |
| | 7.0 | 49 ± 2 | 53 ± 3 | 273 ± 54 | 143 ± 19 |
| | 8.0 | 88 ± 8 | 105 ± 14 | 344 ± 84 | 204 ± 45 |
| | 9.0 | 53 ± 3 | 52 ± 2 | 340 ± 74 | 144 ± 16 |
| | 10.3 | 93 ± 8 | 98 ± 8 | 298 ± 58 | 222 ± 29 |
| | 11.0 | 76 ± 7 | 81 ± 6 | 327 ± 42 | 233 ± 32 |
| | 12.1 | 68 ± 9 | 63 ± 3 | 327 ± 50 | 187 ± 26 |

TABLE 7
Body weight changes from baseline

| | | Isotype | | Isotype | |
|---|---|---|---|---|---|
| | | control in | H4H1327P | control in | H4H1327P in |
| | Time | wild-type | in wild- | Otc mutant | Otc mutant |
| | (weeks) | mice | type mice | mice | mice |
| Body | 0 | 0.0 ± 0.0 | 0.0 ± 0.0 | 0.0 ± 0.0 | 0.0 ± 0.0 |
| weight (% | 0.3 | 2.2 ± 0.4 | 2.7 ± 0.5 | −7.0 ± 1.2 | −7.8 ± 1.0 |
| change | 1.3 | 6.3 ± 0.5 | 5.4 ± 0.3 | −11.8 ± 3.6 | −7.3 ± 3.2 |
| from | 2.1 | 8.6 ± 0.8 | 7.6 ± 0.7 | −14.3 ± 4.0 | −3.9 ± 2.2 |
| baseline) | 3.3 | 11.4 ± 1.0 | 11.5 ± 0.8 | −14.8 ± 4.8 | 4.9 ± 1.6 |
| | 4.3 | 15.3 ± 1.4 | 16.1 ± 1.2 | −13.7 ± 5.0 | 8.5 ± 1.8 |
| | 5.3 | 17.6 ± 1.3 | 17.9 ± 0.9 | −15.3 ± 5.2 | 10.9 ± 1.8 |
| | 6.0 | 19.8 ± 1.7 | 20.6 ± 1.0 | −15.7 ± 4.9 | 12.6 ± 2.1 |
| | 7.0 | 22.9 ± 2.2 | 23.2 ± 0.9 | −15.7 ± 4.9 | 14.5 ± 2.5 |
| | 8.0 | 23.7 ± 2.5 | 23.2 ± 1.3 | −15.8 ± 5.3 | 15.1 ± 2.4 |
| | 9.0 | 27.8 ± 2.2 | 25.6 ± 1.1 | −16.1 ± 7.1 | 15.8 ± 2.6 |
| | 10.3 | 29.4 ± 2.3 | 26.6 ± 0.7 | −16.1 ± 6.6 | 15.9 ± 2.7 |
| | 11.0 | 31.9 ± 2.6 | 27.7 ± 1.3 | −17.6 ± 6.1 | 18.0 ± 2.9 |
| | 12.1 | 34.0 ± 2.7 | 29.3 ± 1.2 | −18.5 ± 7.7 | 20.9 ± 2.3 |

TABLE 8
Blood glucose levels

| | | Isotype | | Isotype | |
|---|---|---|---|---|---|
| | | control in | H4H1327P | control in | H4H1327P in |
| | Time | wild-type | in wild- | Otc mutant | Otc mutant |
| | (weeks) | mice | type mice | mice | mice |
| Blood | 0 | 204 ± 6 | 204 ± 4 | 199 ± 7 | 204 ± 5 |
| glucose | 0.3 | 192 ± 4 | 194 ± 4 | 184 ± 6 | 182 ± 6 |
| (mg/dL) | 1.3 | 184 ± 4 | 120 ± 2 | 148 ± 10 | 95 ± 5 |
| | 2.1 | 181 ± 4 | 127 ± 2 | 165 ± 7 | 116 ± 4 |
| | 3.3 | 173 ± 5 | 123 ± 2 | 155 ± 11 | 119 ± 5 |
| | 4.3 | 170 ± 3 | 130 ± 3 | 138 ± 8 | 126 ± 5 |
| | 5.3 | 167 ± 5 | 130 ± 2 | 137 ± 12 | 118 ± 4 |
| | 6.0 | 172 ± 4 | 131 ± 2 | 150 ± 12 | 125 ± 4 |
| | 7.0 | 164 ± 3 | 128 ± 3 | 127 ± 10 | 125 ± 3 |
| | 8.0 | 168 ± 3 | 134 ± 2 | 132 ± 15 | 121 ± 3 |
| | 9.0 | 171 ± 4 | 133 ± 2 | 133 ± 13 | 123 ± 4 |
| | 10.3 | 177 ± 7 | 140 ± 2 | 144 ± 14 | 129 ± 4 |
| | 11.0 | 165 ± 3 | 135 ± 3 | 129 ± 20 | 122 ± 5 |
| | 12.1 | 168 ± 4 | 147 ± 6 | 130 ± 19 | 124 ± 5 |

TABLE 9
Informal Sequence Listing

| SEQ. | | |
|---|---|---|
| ID NO. | SEQUENCE | DESCRIPTION |
| 1 | caggtccagt tggtacagtc tggggctgac gtgaagaagc ctggggcctc | DNA |
| | agtgaaggtc tcctgcaagg tttccggaca tatcctcact gatttatcca tgcactgggt | nucleotide |
| | gcgacagcct cctggaaaag gacttgagtg gatggcaggt tttgatcctg | sequence |
| | aagaaggtaa aataatctac gcacagaagt tccagggcag agtcaccatg | |
| | accgaggaca catctacaga cacagcctac atggagctga gcagcctgag | |
| | atctggggac acggccgttt attactgtgc aacaagcgat attttgactg ggtattatag | |
| | agactactac ggtttggacg tctggggcca agggaccacg ctcaccgtct cctca | |
| 2 | QVQLVQSGAD VKKPGASVKV SCKVSGHILT | AA amino |
| | DLSMHWVRQP PGKGLEWMAG FDPEEGKIIY | acid |
| | AQKFQGRVTM TEDTSTDTAY MELSSLRSGD | sequence |
| | TAVYYCATSD ILTGYYRDYY GLDVWGQGTT LTVSS | |
| 3 | ggacatatcc tcactgattt atcc | DNA |
| | | nucleotide |
| | | sequence |
| 4 | GHILTDLS | AA amino |
| | | acid |
| | | sequence |
| 5 | tttgatcctg aagaaggtaa aata | DNA |
| | | nucleotide |
| | | sequence |
| 6 | FDPEEGKI | AA amino |
| | | acid |
| | | sequence |
| 7 | gcaacaagcg atattttgac tgggtattat agagactact acggtttgga cgtc | DNA |
| | | nucleotide |
| | | sequence |
| 8 | ATSDILTGYY RDYYGLDV | AA amino |
| | | acid |
| | | sequence |
| 9 | gatattgtga tgactcagtc tccactcttc ctgcccgtca cccctggaga gccggcctcc | DNA |
| | atctcctgca ggtctagtca gagcctcctg catagtaaag gatacaacta tttggattgg | nucleotide |
| | tacctgcaga agccagggca gtctccacaa ctcctgatct atttgggttc taatcgggcc | sequence |
| | tccggggtcc ctgacaggtt cagtggcagt ggatcaggca cagattttac actgaaaatc | |
| | agcagagtgg aggctgaaga tgttggggtt tattactgca tgcaaactct acaaactcct | |
| | cggacgttcg gccaagggac caaggtggaa atcaaa | |
| 10 | DIVMTQSPLF LPVTPGEPAS ISCRSSQSLL HSKGYNYLDW | AA amino |
| | YLQKPGQSPQ LLIYLGSNRA SGVPDRESGS | acid |
| | GSGTDFTLKI SRVEAEDVGV YYCMQTLQTP | sequence |
| | RTFGQGTKVE IK | |
| 11 | cagagcctcc tgcatagtaa aggatacaac tat | DNA |
| | | nucleotide |
| | | sequence |
| 12 | QSLLHSKGYN Y | AA amino |
| | | acid |
| | | sequence |
| 13 | ttgggttct | DNA |
| | | nucleotide |
| | | sequence |
| 14 | LGS | AA amino |
| | | acid |
| | | sequence |
| 15 | atgcaaactc tacaaactcc tcggacg | DNA |
| | | nucleotide |
| | | sequence |
| 16 | MQTLQTPRT | AA amino |
| | | acid |
| | | sequence |
| 17 | caggtccagt tggtacagtc tggggctgac gtgaagaagc ctggggcctc | DNA |
| | agtgaaggtc tcctgcaagg tttccggaca tatcctcact gatttatcca tgcactgggt | nucleotide |
| | gcgacaggct cctggaaaag ggcttgagtg gatgggaggt tttgatcctg | sequence |
| | aagaaggtga aataatctac gcacagaagt tccagggcag agtcaccatg | |
| | accgaggaca catctacaga cacagcctac atggagctga gcagcctgag | |
| | atctggggac acggccgttt attactgtgc aacaagcgat attttgactg gttattatag | |
| | agactactac ggtttggacg tctggggcca agggaccacg ctcaccgtct cctca | |
| 18 | QVQLVQSGAD VKKPGASVKV SCKVSGHILT | AA amino |
| | DLSMHWVRQA PGKGLEWMGG FDPEEGEIIY | acid |
| | AQKFQGRVTM TEDTSTDTAY MELSSLRSGD | sequence |
| | TAVYYCATSD ILTGYYRDYY GLDVWGQGTT LTVS | |
| 19 | ggacatatcc tcactgattt atcc | DNA |
| | | nucleotide |
| | | sequence |
| 20 | GHILTDLS | AA amino |
| | | acid |
| | | sequence |
| 21 | tttgatcctg aagaaggtga aata | DNA |
| | | nucleotide |
| | | sequence |
| 22 | FDPEEGEI | AA amino |
| | | acid |
| | | sequence |
| 23 | gcaacaagcg atattttgac tggttattat agagactact acggtttgga cgtc | DNA |
| | | nucleotide |
| | | sequence |
| 24 | ATSDILTGYY RDYYGLDV | AA amino |
| | | acid |
| | | sequence |
| 25 | atctcctgca ggtctagtca gagcctcctg catagtaaag gatacaacta tttggattgg | DNA |
| | tacctgcaga agccagggca gtctccacaa ctcctgatct atttgggttc taatcgggcc | nucleotide |
| | tccggggtcc ctgacaggtt cagtggcagt ggatcaggca cagattttac actgaaaatc | sequence |
| | agcagagtgg aggctgaaga tgttggggtt tattactgca tgcaaactct acaaactcct | |
| | cggacgttcg gccaagggac caaggtggaa atcaaa | |
| 26 | DIVMTQSPLF LPVTPGEPAS ISCRSSQSLL HSKGYNYLDW | AA amino |
| | YLQKPGQSPQ LLIYLGSNRA SGVPDRESGS GSGTDFTLKI | acid |
| | SRVEAEDVGV YYCMQTLQTP RTFGQGTKVE IK | sequence |
| 27 | cagagcctcc tgcatagtaa aggatacaac tat | DNA |
| | | nucleotide |
| | | sequence |
| 28 | QSLLHSKGYN Y | AA amino |
| | | acid |
| | | sequence |
| 29 | ttgggttct | DNA |
| | | nucleotide |
| | | sequence |
| 30 | LGS | AA amino |
| | | acid |
| | | sequence |
| 31 | atgcaaactc tacaaactcc tcggacg | DNA |
| | | nucleotide |
| | | sequence |
| 32 | MQTLQTPRT | AA amino |
| | | acid |
| | | sequence |
| 33 | gaggagcaac tggtggagtc tgggggagac ttggtacagc ctggagggtc | DNA |
| | cctaagactc tcctgtgcag cctctggatt cactctcagt agttatgaaa tgaactgggt | nucleotide |
| | ccgccaggct ccagggaagg ggctggagtg ggtttcatac attagtagag | sequence |
| | gtggtagtct gatacactac acagactctg tgaagggccg attcaccatc tccagagaca | |
| | acgccaagaa ttcactgtat ctgcaaatga acagcctgag agccgaggac | |
| | acggctgttt attactgtgt gagagaccca gcagctcgtt atcattatta ttatcacggt | |
| | atggacgtct ggggccaagg gaccacggtc accgtctcct ca | |
| 34 | EEQLVESGGD LVQPGGSLRL SCAASGFTLS | AA amino |
| | SYEMNWVRQA PGKGLEWVSY ISRGGSLIHY | acid |
| | TDSVKGRFTI SRDNAKNSLY LQMNSLRAED | sequence |
| | TAVYYCVRDP AARYHYYYHG MDVWGQGTTV TVSS | |
| 35 | ggattcactc tcagtagtta tgaa | DNA |
| | | nucleotide |
| | | sequence |
| 36 | GFTLSSYE | AA amino |
| | | acid |
| | | sequence |
| 37 | attagtagag gtggtagtct gata | DNA |
| | | nucleotide |
| | | sequence |
| 38 | ISRGGSLI | AA amino |
| | | acid |
| | | sequence |
| 39 | gtgagagacc cagcagctcg ttatcattat tattatcacg gtatggacgt c | DNA |
| | | nucleotide |
| | | sequence |
| 40 | VRDPAARYHY YYHGMDV | AA amino |
| | | acid |
| | | sequence |
| 41 | gatattgtga tgactcagtc tccactctcc ctgcccgtca cccctggaga gccggcctcc | DNA |
| | 60atctcctgca ggtctagtca gagcctcctg cacaataatg gatataacta | nucleotide |
| | tttggattgg tatctgcaga agccagggca gtctccacag ctcctgatct atttgggttc | sequence |
| | tagtcgggcc tccggggtcc ctgacaggtt cagtggcagt ggatcaggca cagattttat | |
| | actgaaaatc agcagagtgg aggctgaaga tgttggggtt tattactgca tgcaagctct | |
| | acaaactccg tggacgttcg gccgagggac caaggtggaa atcaaa | |
| 42 | DIVMTQSPLS LPVTPGEPAS ISCRSSQSLL HNNGYNYLDW | AA amino |
| | YLQKPGQSPQ LLIYLGSSRA SGVPDRESGS GSGTDFILKI | acid |
| | SRVEAEDVGV YYCMQALQTP WTFGRGTKVE IK | sequence |
| 43 | cagagcctcc tgcacaataa tggatataac tat | DNA |
| | | nucleotide |
| | | sequence |
| 44 | QSLLHNNGYN Y | AA amino |
| | | acid |
| | | sequence |
| 45 | ttgggttct | DNA |
| | | nucleotide |
| | | sequence |
| 46 | LGS | AA amino |
| | | acid |
| | | sequence |
| 47 | atgcaagctc tacaaactcc gtggacg | DNA |
| | | nucleotide |
| | | sequence |
| 48 | MQALQTPWT | AA amino |
| | | acid |
| | | sequence |
| 49 | gaggtgcagc tggtggagtc tgggggaggc gtggtccagc ctgggaggtc | DNA |
| | cctgagactc tcctgtgcag cctctggatt caccttcagt agttatgaca tgcactgggt | nucleotide |
| | ccgccaggct ccaggcaagg ggctggagtg ggtggcagtt atatcatctg | sequence |
| | atggacgtga taaatactat gtagactccg tgaagggccg attcaccatc tccagagaca | |
| | actccaagaa cacgctttat ctgcaaatga acagcctgag agctgaggac acggctgttt | |
| | attactgtgc gaaagagatg gtgtattacg atattttgac tggttatcat aactactacg | |
| | gtatggacgt ctggggccaa gggaccacgg tcaccgtctc ctca | |
| 50 | EVQLVESGGG VVQPGRSLRL SCAASGFTES | AA amino |
| | SYDMHWVRQA PGKGLEWVAV ISSDGRDKYY | acid |
| | VDSVKGRFTI SRDNSKNTLY LQMNSLRAED | sequence |
| | TAVYYCAKEM VYYDILTGYH NYYGMDVWGQ | |
| | GTTVTVSS | |
| 51 | ggattcacct tcagtagtta tgac | DNA |
| | | nucleotide |
| | | sequence |
| 52 | GFTFSSYD | AA amino |
| | | acid |
| | | sequence |
| 53 | atatcatctg atggacgtga taaa | DNA |
| | | nucleotide |
| | | sequence |
| 54 | ISSDGRDK | AA amino |
| | | acid |
| | | sequence |
| 55 | gcgaaagaga tggtgtatta cgatattttg actggttatc ataactacta cggtatggac | DNA |
| | gtc | nucleotide |
| | | sequence |
| 56 | AKEMVYYDIL TGYHNYYGMD V | AA amino |
| | | acid |
| | | sequence |
| 57 | gacatcgtga tgacccagtc tccatcctca ctgtctgcat ctgtaggaga cagagtcacc | DNA |
| | atcacttgtc gggcgagtca gggcattaac aattatttag cctggtttca gcagaaacca | nucleotide |
| | gggaaagccc ctaagtccct gatccatact gcatccagtt tgcaaagtgg ggtcccatca | sequence |
| | aagttcagcg gcagtggatc tgggacagat ttcactctca ccatcagcag | |
| | cctgcagcct gaagattttg caacttatta ctgccaacag tataatactt accctctcac | |
| | tttcggcgga gggaccaaag tggagatcaa acga | |
| 58 | DIVMTQSPSS LSASVGDRVT ITCRASQGIN | AA amino |
| | NYLAWFQQKP GKAPKSLIHT ASSLQSGVPS | acid |
| | KFSGSGSGTD FTLTISSLQP EDFATYYCQQ YNTYPLTFGG | sequence |
| | GTKVEIKR | |
| 59 | cagggcatta acaattat | DNA |
| | | nucleotide |
| | | sequence |
| 60 | QGINNY | AA amino |
| | | acid |
| | | sequence |
| 61 | actgcatcc | DNA |
| | | nucleotide |
| | | sequence |
| 62 | TAS | AA amino |
| | | acid |
| | | sequence |
| 63 | caacagtata atacttaccc tctcact | DNA |
| | | nucleotide |
| | | sequence |
| 64 | QQYNTYPLT | AA amino |
| | | acid |
| | | sequence |
| 65 | caggtgcagc tggtggagtc tgggggaggc gtggtccagc ctgggaggtc | DNA |
| | cctgagactc tcctgtgcag cctctggatt caccttcagt agttatgaca tgcactgggt | nucleotide |
| | ccgccaggct ccaggcaagg ggctggagtg ggtggcagtt atatcatctg | sequence |
| | atggacgtga taaatactat gtagactccg tgaagggccg attcaccatc tccagagaca | |
| | actccaagaa cacgctttat ctgcaaatga acagcctgag agctgaggac acggctgttt | |
| | attactgtgc gaaagagatg gtgtattacg atattttgac tggttatcat aactactacg | |
| | gtatggacgt ctggggccaa gggaccacgg tcaccgtctc c | |
| 66 | QVQLVESGGG VVQPGRSLRL SCAASGFTFS | AA amino |
| | SYDMHWVRQA PGKGLEWVAV ISSDGRDKYY | acid |
| | VDSVKGRFTI SRDNSKNTLY LQMNSLRAED | sequence |
| | TAVYYCAKEM VYYDILTGYH NYYGMDVWGQ | |
| | GTTVTVS | |
| 67 | gacatccaga tgacccagtc tccatcctca ctgtctgcat ctgtaggaga cagagtcacc | DNA |
| | atcacttgtc gggcgagtca gggcattaac aattatttag cctggtttca gcagaaacca | nucleotide |
| | gggaaagccc ctaagtccct gatccatact gcatccagtt tgcaaagtgg ggtcccatca | sequence |
| | aagttcagcg gcagtggatc tgggacagat ttcactctca ccatcagcag | |
| | cctgcagcct gaagattttg caacttatta ctgccaacag tataatactt accctctcac | |
| | tttcggcgga gggaccaagg tggagatcaa a | |
| 68 | DIQMTQSPSS LSASVGDRVT ITCRASQGIN | AA amino |
| | NYLAWFQQKP GKAPKSLIHT ASSLQSGVPS | acid |
| | KFSGSGSGTD FTLTISSLQP EDFATYYCQQ YNTYPLTFGG | sequence |
| | GTKVEIK | |
| 69 | caggtgcagc tggtgcagtc tgggggaggc ttggtccagc ctggggggtc | DNA |
| | cctgagactc tcctgtgcag cctccggatt cacctttagt aactatttga tgaactgggt | nucleotide |
| | ccgccaggct ccagggaagg ggctggagtg gctggccaac atacaggaag | sequence |
| | atggaattga gaaatactat gtggactctg tgaagggccg attcaccatc tccagagaca | |
| | acgccaagaa ctcactgtat ctgcaaatga acagcctgag agccgaggac | |
| | acggctgtgt attactgtgc gagagagccc tcccattacg atattttgac tggttatgac | |
| | tactattacg gtatggacgt ctggggccaa gggaccacgg tcaccgtctc ctca | |
| 70 | QVQLVQSGGG LVQPGGSLRL SCAASGFTFS | AA amino |
| | NYLMNWVRQA PGKGLEWLAN IQEDGIEKYY | acid |
| | VDSVKGRFTI SRDNAKNSLY LQMNSLRAED | sequence |
| | TAVYYCAREP SHYDILTGYD YYYGMDVWGQ | |
| | GTTVTVSS | |
| 71 | ggattcacct ttagtaacta tttg | DNA |
| | | nucleotide |
| | | sequence |
| 72 | GFTFSNYL | AA amino |
| | | acid |
| | | sequence |
| 73 | atacaggaag atggaattga gaaa | DNA |
| | | nucleotide |
| | | sequence |
| 74 | IQEDGIEK | AA amino |
| | | acid |
| | | sequence |
| 75 | gcgagagagc cctcccatta cgatattttg actggttatg actactatta cggtatggac | DNA |
| | gtc | nucleotide |
| | | sequence |
| 76 | AREPSHYDIL TGYDYYYGMD V | AA amino |
| | | acid |
| | | sequence |
| 77 | gacatccagt tgacccagtc tccatcctcc ctgtctgcat ctgtaggaga cagagtcacc | DNA |
| | atcacttgcc gggcaagtca gggcattaga aatgatttag gctggtatca | nucleotide |
| | gcagaaacca gggaaagccc ctaagcgcct gatctatgct gcatccagtt | sequence |
| | tgcaaagtgg ggtcccatca aggttcagcg gcagtggatc tgggacagaa | |
| | ttcattctca cagtcagcag cctgcagcct gaagactttg caacttatta ctgtctacag | |
| | tataatagta acccattcac tttcggccct gggaccaagg tggagatcaa acga | |
| 78 | DIQLTQSPSS LSASVGDRVT ITCRASQGIR NDLGWYQQKP | AA amino |
| | GKAPKRLIYA ASSLQSGVPS RFSGSGSGTE FILTVSSLQP | acid |
| | EDFATYYCLQ YNSNPFTFGP GTKVEIKR | sequence |
| 79 | cagggcatta gaaatgat | DNA |
| | | nucleotide |
| | | sequence |
| 80 | QGIRND | AA amino |
| | | acid |
| | | sequence |
| 81 | gctgcatcc | DNA |
| | | nucleotide |
| | | sequence |
| 82 | AAS | AA amino |
| | | acid |
| | | sequence |
| 83 | ctacagtata atagtaaccc attcact | DNA |
| | | nucleotide |
| | | sequence |
| 84 | LQYNSNPFT | AA amino |
| | | acid |
| | | sequence |
| 85 | gaggtgcagc tggtggagtc tgggggaggc ttggtccagc ctggggggtc | DNA |
| | cctgagactc tcctgtgcag cctccggatt cacctttagt aactatttga tgaactgggt | nucleotide |
| | ccgccaggct ccagggaagg ggctggagtg gctggccaac atacaggaag | sequence |
| | atggaattga gaaatactat gtggactctg tgaagggccg attcaccatc tccagagaca | |
| | acgccaagaa ctcactgtat ctgcaaatga acagcctgag agccgaggac | |
| | acggctgtgt attactgtgc gagagagccc tcccattacg atattttgac tggttatgac | |
| | tactattacg gtatggacgt ctggggccaa gggaccacgg tcaccgtctc c | |
| 86 | EVQLVESGGG LVQPGGSLRL SCAASGFTFS | AA amino |
| | NYLMNWVRQA PGKGLEWLAN IQEDGIEKYY | acid |
| | VDSVKGRFTI SRDNAKNSLY LQMNSLRAED | sequence |
| | TAVYYCAREP SHYDILTGYD YYYGMDVWGQ | |
| | GTTVTVS | |
| 87 | gacatccaga tgacccagtc tccatcctcc ctgtctgcat ctgtaggaga cagagtcacc | DNA |
| | atcacttgcc gggcaagtca gggcattaga aatgatttag gctggtatca | nucleotide |
| | gcagaaacca gggaaagccc ctaagcgcct gatctatgct gcatccagtt | sequence |
| | tgcaaagtgg ggtcccatca aggttcagcg gcagtggatc tgggacagaa | |
| | ttcattctca cagtcagcag cctgcagcct gaagactttg caacttatta ctgtctacag | |
| | tataatagta acccattcac tttcggccct gggaccaaag tggatatcaa a | |
| 88 | DIQMTQSPSS LSASVGDRVT ITCRASQGIR | AA amino |
| | NDLGWYQQKP GKAPKRLIYA ASSLQSGVPS | acid |
| | RFSGSGSGTE FILTVSSLQP EDFATYYCLQ YNSNPFTFGP | sequence |
| | GTKVDIK | |
| 89 | tcctgtacag cctctggttt caccttcagt aactacgaca tgcactgggt ccgccaaact | DNA |
| | acaggaaaag gtctggagtg gatctcagct attgatactg ctggtgacac atactatcca | nucleotide |
| | ggctccgtga agggccgatt caccgtctcc agagaaaatg ccaagaactc cttttatctt | sequence |
| | caaatgaaca gcctgagagc cggggacacg gctgtgtatt actgtgcaag | |
| | ggaggggaag tattacgata ttttgactgg tgactaccac tactacggta tggacgtctg | |
| | gggccaaggg accacggtca ccgtctcctc a | |
| 90 | EVQLVQSGGA LVQPGGSLRL SCTASGFTFS | AA amino |
| | NYDMHWVRQT TGKGLEWISA IDTAGDTYYP | acid |
| | GSVKGRFTVS RENAKNSFYL QMNSLRAGDT | sequence |
| | AVYYCAREGK YYDILTGDYH YYGMDVWGQG | |
| | TTVTVSS | |
| 91 | ggtttcacct tcagtaacta cgac | DNA |
| | | nucleotide |
| | | sequence |
| 92 | GFTFSNYD | AA amino |
| | | acid |
| | | sequence |
| 93 | attgatactg ctggtgacac a | DNA |
| | | nucleotide |
| | | sequence |
| 94 | IDTAGDT | AA amino |
| | | acid |
| | | sequence |
| 95 | gcaagggagg ggaagtatta cgatattttg actggtgact accactacta cggtatggac | DNA |
| | 60gtc | nucleotide |
| | | sequence |
| 96 | AREGKYYDIL TGDYHYYGMD V | AA amino |
| | | acid |
| | | sequence |
| 97 | atcacttgtc gggcaagtca gggcattaga aatgatttag gctggtatca gcagaaacca | DNA |
| | gggaaagccc ctaagcgact gatctatgct acatccagtt tgcaaagtgg | nucleotide |
| | ggtcccatca aggttcagcg gcagtggatc tgggacagaa ttcactctca | sequence |
| | caatcagcag cctgcagcct gaagattttg caacttatta ctgtctacag cataatagtt | |
| | acccgctcac tttcggcgga gggaccaagg tggaaatcaa acga | |
| 98 | AIRMTQSPSS LSASVGDRVT ITCRASQGIR | AA amino |
| | NDLGWYQQKP GKAPKRLIYA TSSLQSGVPS | acid |
| | RFSGSGSGTE FTLTISSLQP EDFATYYCLQ HNSYPLTFGG | sequence |
| | GTKVEIKR | |
| 99 | cagggcatta gaaatgat | DNA |
| | | nucleotide |
| | | sequence |
| 100 | QGIRND | AA amino |
| | | acid |
| | | sequence |
| 101 | gctacatcc | DNA |
| | | nucleotide |
| | | sequence |
| 102 | ATS | AA amino |
| | | acid |
| | | sequence |
| 103 | ctacagcata atagttaccc gctcact | DNA |
| | | nucleotide |
| | | sequence |
| 104 | LQHNSYPLT | AA amino |
| | | acid |
| | | sequence |
| 105 | gaggtgcagc tggtggagtc tgggggagcc ttggtacagc ctggggggtc | DNA |
| | cctgagactc tcctgtacag cctctggttt caccttcagt aactacgaca tgcactgggt | nucleotide |
| | ccgccaaact acaggaaaag gtctggagtg gatctcagct attgatactg | sequence |
| | ctggtgacac atactatcca ggctccgtga agggccgatt caccgtctcc | |
| | agagaaaatg ccaagaactc cttttatctt caaatgaaca gcctgagagc | |
| | cggggacacg gctgtgtatt actgtgcaag ggaggggaag tattacgata ttttgactgg | |
| | tgactaccac tactacggta tggacgtctg gggccaaggg accacggtca ccgtctcc | |
| 106 | EVQLVESGGA LVQPGGSLRL SCTASGFTFS | AA amino |
| | NYDMHWVRQT TGKGLEWISA IDTAGDTYYP | acid |
| | GSVKGRFTVS RENAKNSFYL QMNSLRAGDT | sequence |
| | AVYYCAREGK YYDILTGDYH YYGMDVWGQG | |
| | TTVTVS | |
| 107 | gacatccaga tgacccagtc tcatcctcc ctgtctgcat ctgtaggaga cagagtcacc | DNA |
| | atcacttgtc gggcaagtca gggcattaga aatgatttag gctggtatca gcagaaacca | nucleotide |
| | gggaaagccc ctaagcgact gatctatgct acatccagtt tgcaaagtgg | sequence |
| | ggtcccatca aggttcagcg gcagtggatc tgggacagaa ttcactctca | |
| | caatcagcag cctgcagcct gaagattttg caacttatta ctgtctacag cataatagtt | |
| | acccgctcac tttcggcgga gggaccaagc tggagatcaa a | |
| 108 | DIQMTQSPSS LSASVGDRVT ITCRASQGIR | AA amino |
| | NDLGWYQQKP GKAPKRLIYA TSSLQSGVPS | acid |
| | RFSGSGSGTE FTLTISSLQP EDFATYYCLQ HNSYPLTFGG | sequence |
| | GTKLEIK | |
| 109 | caggtgcagc tggtgcagtc tgggggaggc gtggtccagc ctgggaggtc | DNA |
| | cctgagactc tcctgtgcag cgtctgggtt cacctttagt aactttggca tgcactgggt | nucleotide |
| | ccgccaggct ccaggcaagg ggctggagtg ggtggcagtt atatggtttg | sequence |
| | atgaaattga taaatactat gcagactccg tgaagggccg attcaccatc tccagagaca | |
| | attccaagaa cacgctgtat ccgcaaatga acagcctgag agccgaagac | |
| | acggctgtgt attactgtgc gcgagaagat tacgatattt tgactggtta ctattacgct | |
| | atggacgtct ggggccaagg gaccacggtc accgtctcct ca | |
| 110 | QVQLVQSGGG VVQPGRSLRL SCAASGFTFS | AA amino |
| | NFGMHWVRQA PGKGLEWVAV IWFDEIDKYY | acid |
| | ADSVKGRFTI SRDNSKNTLY PQMNSLRAED | sequence |
| | TAVYYCARED YDILTGYYYA MDVWGQGTTV TVSS | |
| 111 | gggttcacct ttagtaactt tggc | DNA |
| | | nucleotide |
| | | sequence |
| 112 | GFTFSNFG | AA amino |
| | | acid |
| | | sequence |
| 113 | atatggtttg atgaaattga taaa | DNA |
| | | nucleotide |
| | | sequence |
| 114 | IWFDEIDK | AA amino |
| | | acid |
| | | sequence |
| 115 | gcgcgagaag attacgatat tttgactggt tactattacg ctatggacgt c | DNA |
| | | nucleotide |
| | | sequence |
| 116 | AREDYDILTG YYYAMDV | AA amino |
| | | acid |
| | | sequence |
| 117 | gacatccaga tgacccagtc tccatcctcc ctgtctgcat ctgtaggaga cagagtcacc | DNA |
| | atcacttgcc gggcaagtca gggcattaga aatgatttag gctggtatca | nucleotide |
| | gcagaaacca gggaaagccc ctaagcgcct aatctatgct gcatcccgtt | sequence |
| | tgcaaagtgg ggtcccatcg aggttcagcg gcagtggatc tgggacagaa | |
| | ttcactctca caatcagcag cctgcagcct gaagattttg gaacttatta ctgtctacag | |
| | cataatagtc accccacctt cggccaaggg accaaggtgg agatcaaacg a | |
| 118 | DIQMTQSPSS LSASVGDRVT ITCRASQGIR | AA amino |
| | NDLGWYQQKP GKAPKRLIYA ASRLQSGVPS | acid |
| | RFSGSGSGTE FTLTISSLQP EDFGTYYCLQ HNSHPTFGQG | sequence |
| | TKVEIKR | |
| 119 | cagggcatta gaaatgat | DNA |
| | | nucleotide |
| | | sequence |
| 120 | QGIRND | AA amino |
| | | acid |
| | | sequence |
| 121 | gctgcatcc | DNA |
| | | nucleotide |
| | | sequence |
| 122 | AAS | AA amino |
| | | acid |
| | | sequence |
| 123 | ctacagcata atagtcaccc cacc | DNA |
| | | nucleotide |
| | | sequence |
| 124 | LQHNSHPT | AA amino |
| | | acid |
| | | sequence |
| 125 | caggtgcagc tggtggagtc tgggggaggc gtggtccagc ctgggaggtc | DNA |
| | cctgagactc tcctgtgcag cgtctgggtt cacctttagt aactttggca tgcactgggt | nucleotide |
| | ccgccaggct ccaggcaagg ggctggagtg ggtggcagtt atatggtttg | sequence |
| | atgaaattga taaatactat gcagactccg tgaagggccg attcaccatc tccagagaca | |
| | attccaagaa cacgctgtat ccgcaaatga acagcctgag agccgaagac | |
| | acggctgtgt attactgtgc gcgagaagat tacgatattt tgactggtta ctattacgct | |
| | atggacgtct ggggccaagg gaccacggtc accgtctcc | |
| 126 | QVQLVESGGG VVQPGRSLRL SCAASGFTFS | AA amino |
| | NFGMHWVRQA PGKGLEWVAV IWFDEIDKYY | acid |
| | ADSVKGRFTI SRDNSKNTLY PQMNSLRAED | sequence |
| | TAVYYCARED YDILTGYYYA MDVWGQGTTV TVS | |
| 127 | gacatccaga tgacccagtc tccatcctcc ctgtctgcat ctgtaggaga cagagtcacc | DNA |
| | atcacttgcc gggcaagtca gggcattaga aatgatttag gctggtatca | nucleotide |
| | gcagaaacca gggaaagccc ctaagcgcct aatctatgct gcatcccgtt | sequence |
| | tgcaaagtgg ggtcccatcg aggttcagcg gcagtggatc tgggacagaa | |
| | ttcactctca caatcagcag cctgcagcct gaagattttg gaacttatta ctgtctacag | |
| | cataatagtc accccacctt cggccaaggg accaaggtgg agatcaaa | |
| 128 | DIQMTQSPSS LSASVGDRVT ITCRASQGIK | AA amino |
| | NDLGWYQQKP GKAPKRLIYA ASRLQSGVPS | acid |
| | RFSGSGSGTE FTLTISSLQP EDFGTYYCLQ HNSHPTFGQG | sequence |
| | TKVEIK | |
| 129 | gaggtgcagc tggtggagtc ggggggaggc atggtacagc ctggggggtc | DNA |
| | cctgagactc tcctgtgcag cctctggatt cacctccagt aactacgaca tgcactgggt | nucleotide |
| | ccgccaagct acaggaaaag gtctggagtg ggtctcaagt attgatactg | sequence |
| | ctggggacac ttactatcca gactccgtga agggccgctt tatcatctcc | |
| | agagaaaatg ccaaaaactc cctgtatctt caaatgaata gcctgagagc | |
| | cggggacacg gctgtgtatt actgtacaag ggagccccga aattacgaaa | |
| | ttttgactgg tcactaccac taccacggta tggacatctg gggccaaggg | |
| | accacggtca ccgtctcctc a | |
| 130 | EVQLVESGGG MVQPGGSLRL SCAASGFTSS | AA amino |
| | NYDMHWVRQA TGKGLEWVSS IDTAGDTYYP | acid |
| | DSVKGRFIIS RENAKNSLYL QMNSLRAGDT | sequence |
| | AVYYCTREPR NYEILTGHYH YHGMDIWGQG TTVTVSS | |
| 131 | ggattcacct ccagtaacta cgac | DNA |
| | | nucleotide |
| | | sequence |
| 132 | GFTSSNYD | AA amino |
| | | acid |
| | | sequence |
| 133 | attgatactg ctggggacac t | DNA |
| | | nucleotide |
| | | sequence |
| 134 | IDTAGDT | AA amino |
| | | acid |
| | | sequence |
| 135 | acaagggagc cccgaaatta cgaaattttg actggtcact accactacca | DNA |
| | cggtatggac atc | nucleotide |
| | | sequence |
| 136 | TREPRNYEIL TGHYHYHGMD I | AA amino |
| | | acid |
| | | sequence |
| 137 | gacatccaga tgacccagtc gccatcctcc ctgtctgcat ctgtaggaga | DNA |
| | cagagtcacc atcacttgcc gggcaagtca ggccattaga aatgatttag gctggtatca | nucleotide |
| | gcagaaacca gggaaagccc ctaaactcct gatctatact gcattcagtt tacagagtgg | sequence |
| | ggtcccatca aggttcagcg gcagtaaatc tggcacagac ttcactctca | |
| | ccatcagcag cctgcagcct gaagattttg cgacttatta ctgtctgcag gattacacta | |
| | atcctcggac gttcggccaa gggaccaagg tggagatcaa acga | |
| 138 | DIQMTQSPSS LSASVGDRVT ITCRASQAIR | AA amino |
| | NDLGWYQQKP GKAPKLLIYT AFSLQSGVPS | acid |
| | RFSGSKSGTD FTLTISSLQP EDFATYYCLQ DYTNPRTFGQ | sequence |
| | GTKVEIKR | |
| 139 | caggccatta gaaatgat | DNA |
| | | nucleotide |
| | | sequence |
| 140 | QAIRND | AA amino |
| | | acid |
| | | sequence |
| 141 | actgcattc | DNA |
| | | nucleotide |
| | | sequence |
| 142 | TAF | AA amino |
| | | acid |
| | | sequence |
| 143 | ctgcaggatt acactaatcc tcggacg | DNA |
| | | nucleotide |
| | | sequence |
| 144 | LQDYTNPRT | AA amino |
| | | acid |
| | | sequence |
| 145 | gaggtgcagc tggtggagtc ggggggaggc atggtacagc ctggggggtc | DNA |
| | cctgagactc tcctgtgcag cctctggatt cacctccagt aactacgaca tgcactgggt | nucleotide |
| | ccgccaagct acaggaaaag gtctggagtg ggtctcaagt attgatactg | sequence |
| | ctggggacac ttactatcca gactccgtga agggccgctt tatcatctcc agagaaaatg | |
| | ccaaaaactc cctgtatctt caaatgaata gcctgagagc cggggacacg | |
| | gctgtgtatt actgtacaag ggagccccga aattacgaaa ttttgactgg | |
| | tcactaccac taccacggta tggacatctg gggccaaggg accacggtca ccgtctcc | |
| 146 | EVQLVESGGG MVQPGGSLRL SCAASGFTSS | AA amino |
| | NYDMHWVRQA TGKGLEWVSS IDTAGDTYYP | acid |
| | DSVKGRFIIS RENAKNSLYL QMNSLRAGDT | sequence |
| | AVYYCTREPR NYEILTGHYH YHGMDIWGQG TTVTVS | |
| 147 | gccatccaga tgacccagtc gccatcctcc ctgtctgcat ctgtaggaga | DNA |
| | cagagtcacc atcacttgcc gggcaagtca ggccattaga aatgatttag gctggtatca | nucleotide |
| | gcagaaacca gggaaagccc ctaaactcct gatctatact gcattcagtt tacagagtgg | sequence |
| | ggtcccatca aggttcagcg gcagtaaatc tggcacagac ttcactctca | |
| | ccatcagcag cctgcagcct gaagattttg cgacttatta ctgtctgcag gattacacta | |
| | atcctcggac gttcggccaa gggaccaagg tggaaatcaa a | |
| 148 | AIQMTQSPSS LSASVGDRVT ITCRASQAIR | AA amino |
| | NDLGWYQQKP GKAPKLLIYT AFSLQSGVPS | acid |
| | RFSGSKSGTD FTLTISSLQP EDFATYYCLQ DYTNPRTFGQ | sequence |
| | GTKVEIK | |
| 149 | caggtgcagc tggtggagtc tgggggaggc gtggtccagc ctgggaggtc | DNA |
| | cctgagactc tcctgtgcag cctctggatt cgccttcagt aactatggca tgcactgggt | nucleotide |
| | ccgccaggct ccaggcaagg ggctggaatg ggtgacattt atatcatatg | sequence |
| | atggaagtaa taaatactat gcagactccg tgaagggccg attcaccatc | |
| | tccagagaca attccaagaa cacgctgtat ctgcaagtga acagcctgag | |
| | agctgaggac acggctgtgt attactgtgc gaaagaagca gtattagctg ccctctttga | |
| | ctactggggc cagggaaccc tggtcaccgt ctcctca | |
| 150 | QVQLVESGGG VVQPGRSLRL SCAASGFAFS | AA amino |
| | NYGMHWVRQA PGKGLEWVTF ISYDGSNKYY | acid |
| | ADSVKGRFTI SRDNSKNTLY LQVNSLRAED | sequence |
| | TAVYYCAKEA VLAALFDYWG QGTLVTVSS | |
| 151 | ggattcgcct tcagtaacta tggc | DNA |
| | | nucleotide |
| | | sequence |
| 152 | GFAFSNYG | AA amino |
| | | acid |
| | | sequence |
| 153 | atatcatatg atggaagtaa taaa | DNA |
| | | nucleotide |
| | | sequence |
| 154 | ISYDGSNK | AA amino |
| | | acid |
| | | sequence |
| 155 | gcgaaagaag cagtattagc tgccctcttt gactac | DNA |
| | | nucleotide |
| | | sequence |
| 156 | AKEAVLAALF DY | AA amino |
| | | acid |
| | | sequence |
| 157 | gacatcgtga tgacccagtc tccagactcc ctggctgtgt ctctgggcga | DNA |
| | gagggccacc atcaactgca agtccagcca gagtgtttta tacagctcca | nucleotide |
| | acaatcagaa ctacttagct tggtaccagc agaaaccagg acagcctcct | sequence |
| | aagctgctca tttactgggc atctacccgg gaatccgggg tccctgaccg | |
| | attcagtggc agcgggtctg ggacagattt cactctcacc atcaacagcc | |
| | tgcaggctga agatgtggca gtttattact gtcagcaata ttatagtact cctacgttcg | |
| | gccaagggac caaggtggaa atcaaa | |
| 158 | DIVMTQSPDS LAVSLGERAT INCKSSQSVL | AA amino |
| | YSSNNQNYLA WYQQKPGQPP KLLIYWASTR | acid |
| | ESGVPDRFSG SGSGTDFTLT INSLQAEDVA | sequence |
| | VYYCQQYYST PTFGQGTKVE IK | |
| 159 | cagagtgttt tatacagctc caacaatcag aactac | DNA |
| | | nucleotide |
| | | sequence |
| 160 | QSVLYSSNNQ NY | AA amino |
| | | acid |
| | | sequence |
| 161 | tgggcatct | DNA |
| | | nucleotide |
| | | sequence |
| 162 | WAS | AA amino |
| | | acid |
| | | sequence |
| 163 | cagcaatatt atagtactcc tacg | DNA |
| | | nucleotide |
| | | sequence |
| 164 | QQYYSTPT | AA amino |
| | | acid |
| | | sequence |
| 165 | caggtgcagc tggtggagtc tgggggaggc gtggtccagc ctgggaggtc | DNA |
| | cctgagactc tcctgtgcag cctctggatt cacgttcaat acctatggca tgcactgggt | nucleotide |
| | ccgccaggct ccagtcaagg ggctggagtg ggtggcattt atatcaaatg | sequence |
| | ataagagtaa tacattctat gcagactccg tgaagggccg attcaccatc tccagagaca | |
| | attccaagaa cacgctgttt ctggaaatga acagcctgac agctgaggac acggctgttt | |
| | attactgtgc gaaagagtcc attttagcag ccctctttga ctactggggc cagggaaccc | |
| | tggtcactgt ctcctca | |
| 166 | QVQLVESGGG VVQPGRSLRL SCAASGFTFN | AA amino |
| | TYGMHWVRQA PVKGLEWVAF ISNDKSNTFY | acid |
| | ADSVKGRFTI SRDNSKNTLF LEMNSLTAED | sequence |
| | TAVYYCAKES ILAALFDYWG QGTLVTVSS | |
| 167 | ggattcacgt tcaataccta tggc | DNA |
| | | nucleotide |
| | | sequence |
| 168 | GFTFNTYG | AA amino |
| | | acid |
| | | sequence |
| 169 | atatcaaatg ataagagtaa taca | DNA |
| | | nucleotide |
| | | sequence |
| 170 | ISNDKSNT | AA amino |
| | | acid |
| | | sequence |
| 171 | gcgaaagagt ccattttagc agccctcttt gactac | DNA |
| | | nucleotide |
| | | sequence |
| 172 | AKESILAALF DY | AA amino |
| | | acid |
| | | sequence |
| 173 | gacatcgtga tgacccagtc tccagactcc ctggctgtgt ctctgggcga | DNA |
| | gagggccacc atcaactgca agtccagcca gagtgtttta tacagctcca | nucleotide |
| | acaataagaa ttacttagct tggtaccaac agaaaccaag acagcctctt aaactactca | sequence |
| | tttactgggc atctattcgg gaatccgggg tccctgaccg attcagtggc agcgggtctg | |
| | ggacagattt cactctcacc atcagcagcc tgcaggctga agatgtggca gtttattact | |
| | gtcagcaatt ttatagtgtt cccacttttg gccaggggac caagctggag atcaaa | |
| 174 | DIVMTQSPDS LAVSLGERAT INCKSSQSVL | AA amino |
| | YSSNNKNYLA WYQQKPRQPL KLLIYWASIR | acid |
| | ESGVPDRFSG SGSGTDFTLT ISSLQAEDVA VYYCQQFYSV | sequence |
| | PTFGQGTKLE IK | |
| 175 | cagagtgttt tatacagctc caacaataag aattac | DNA |
| | | nucleotide |
| | | sequence |
| 176 | QSVLYSSNNK NY | AA amino |
| | | acid |
| | | sequence |
| 177 | tgggcatct | DNA |
| | | nucleotide |
| | | sequence |
| 178 | WAS | AA amino |
| | | acid |
| | | sequence |
| 179 | cagcaatttt atagtgttcc cact | DNA |
| | | nucleotide |
| | | sequence |
| 180 | QQFYSVPT | AA amino |
| | | acid |
| | | sequence |
| 181 | caggtgcagc tggtggagtc tgggggaggc gtggtccagc ctgggaggtc | DNA |
| | cctgagactc tcctgtgcag cctctggatt cacgtttagt acctttggca tgcactgggt | nucleotide |
| | ccgccaggct ccagtcaagg ggctggagtg ggtggctttt atatcaaatg | sequence |
| | ataagaataa taaattctat gcagactccg tgaagggccg attcaccatc tccagagaca | |
| | attccaggga cacgctatat ctgcaaatga acagcctgac acctgaggac acggctgttt | |
| | attactgtgc gaaagagtcc attttagcag ccctctttga ctactggggc cagggaaccc | |
| | tggtcaccgt ctcctca | |
| 182 | QVQLVESGGG VVQPGRSLRL SCAASGFTFS | AA amino |
| | TFGMHWVRQA PVKGLEWVAF ISNDKNNKFY | acid |
| | ADSVKGRFTI SRDNSRDTLY LQMNSLTPED | sequence |
| | TAVYYCAKES ILAALFDYWG QGTLVTVSS | |
| 183 | ggattcacgt ttagtacctt tggc | DNA |
| | | nucleotide |
| | | sequence |
| 184 | GFTFSTFG | AA amino |
| | | acid |
| | | sequence |
| 185 | atatcaaatg ataagaataa taaa | DNA |
| | | nucleotide |
| | | sequence |
| 186 | ISNDKNNK | AA amino |
| | | acid |
| | | sequence |
| 187 | gcgaaagagt ccattttagc agccctcttt gactac | DNA |
| | | nucleotide |
| | | sequence |
| 188 | AKESILAALF DY | AA amino |
| | | acid |
| | | sequence |
| 189 | gacatcgtga tgacccagtc tccagactcc ctggctgtgt ctctgggcga | DNA |
| | gagggccacc atcaactgca agtccagcca gagtgtttta tacagctcca | nucleotide |
| | acaataaaaa ttacttagct tggtaccagc agaaaccagg acagcctctt aaacttctca | sequence |
| | tttactgggc atctattcgg gaatccgggg tccctgaccg attcagtggc agcgggtctg | |
| | ggacagattt cactctcacc atcagcagcc tgcaggctga agatgtggca gtttattact | |
| | gtcagcaatt ttatactgtt cccacttttg gcctggggac caagctggag atcaaa | |
| 190 | DIVMTQSPDS LAVSLGERAT INCKSSQSVL | AA amino |
| | YSSNNKNYLA WYQQKPGQPL KLLIYWASIR | acid |
| | ESGVPDRFSG SGSGTDFTLT ISSLQAEDVA | sequence |
| | VYYCQQFYTV PTFGLGTKLE IK | |
| 191 | cagagtgttt tatacagctc caacaataaa aattac | DNA |
| | | nucleotide |
| | | sequence |
| 192 | QSVLYSSNNK NY | AA amino |
| | | acid |
| | | sequence |
| 193 | tgggcatct | DNA |
| | | nucleotide |
| | | sequence |
| 194 | WAS | AA amino |
| | | acid |
| | | sequence |
| 195 | cagcaatttt atactgttcc cact | DNA |
| | | nucleotide |
| | | sequence |
| 196 | QQFYTVPT | AA amino |
| | | acid |
| | | sequence |
| 197 | caggtgcagc tggtggagtc tgggggaggc gtggtccagc ctgggaggtc | DNA |
| | cctgagactc tcctgtgtag cctctggatt caccttcagg aactatgaca tgcactgggt | nucleotide |
| | ccgccaggct cctggcaagg ggctggaatg ggtggcagtt acatcatctg | sequence |
| | atggacttaa taaattctat tcagactccg tgaagggccg attcaccatc tccagagaca | |
| | attccaagaa cacgctgtct ctgcaaatta ccggcctgag agctgaggac | |
| | acggctgtgt attactgtgc gaaagagtcc attttagcag ccctctttga ctactggggc | |
| | cagggaaccc tggtcaccgt ctcctca | |
| 198 | QVQLVESGGG VVQPGRSLRL SCVASGFTFR | AA amino |
| | NYDMHWVRQA PGKGLEWVAV TSSDGLNKFY | acid |
| | SDSVKGRFTI SRDNSKNTLS LQITGLRAED TAVYYCAKES | sequence |
| | ILAALFDYWG QGTLVTVSS | |
| 199 | ggattcacct tcaggaacta tgac | DNA |
| | | nucleotide |
| | | sequence |
| 200 | GFTFRNYD | AA amino |
| | | acid |
| | | sequence |
| 201 | acatcatctg atggacttaa taaa | DNA |
| | | nucleotide |
| | | sequence |
| 202 | TSSDGLNK | AA amino |
| | | acid |
| | | sequence |
| 203 | gcgaaagagt ccattttagc agccctcttt gactac | DNA |
| | | nucleotide |
| | | sequence |
| 204 | AKESILAALF DY | AA amino |
| | | acid |
| | | sequence |
| 205 | acatcgtga tgacccagtc tccagactcc ctggctgtgt ctctgggcga gagggccacc | DNA |
| | atcaactgca agtccagcca gagtgtttta tacagctcca acaataagaa ctacttggct | nucleotide |
| | tggtaccagc agaaaccagg acagcctcct aagctgctct tttactgggc atctacccgg | sequence |
| | gaatccgggg tccctgaccg attcagtggc agcgggtctg ggacagattt | |
| | cactctcacc atcagcagcc tgcaggctga agatgtggca gtttattact gtcagcaaca | |
| | ttatactact cccacttttg gccaggggac caagctggag atcaaa | |
| 206 | DIVMTQSPDS LAVSLGERAT INCKSSQSVL | AA amino |
| | YSSNNKNYLA WYQQKPGQPP KLLFYWASTR | acid |
| | ESGVPDRFSG SGSGTDFTLT ISSLQAEDVA | sequence |
| | VYYCQQHYTT PTFGQGTKLE IK | |
| 207 | cagagtgttt tatacagctc caacaataag aactac | DNA |
| | | nucleotide |
| | | sequence |
| 208 | QSVLYSSNNK NY | AA amino |
| | | acid |
| | | sequence |
| 209 | tgggcatct | DNA |
| | | nucleotide |
| | | sequence |
| 210 | WAS | AA amino |
| | | acid |
| | | sequence |
| 211 | cagcaacatt atactactcc cact | DNA |
| | | nucleotide |
| | | sequence |
| 212 | QQHYTTPT | AA amino |
| | | acid |
| | | sequence |
| 213 | caggtgcagc tggtggagtc tgggggaggc gtggtccagc ctgggaggtc | DNA |
| | cctgagactc tcctgtgcag cctctggatt caccttcagt agctatggca tgcactgggt | nucleotide |
| | ccgccagact ccgggcaagg ggctggagtg ggtggcattt atatcatatg | sequence |
| | atggaaataa taaatactat gcagactccg tgaagggccg attcaccatc | |
| | tccagagaca attccaagaa cacgctgttt ctgcaaatga acagcctgag | |
| | agctgaggac acggctgtgt attactgtgc gaaagagtcc attttagcag ccctctttga | |
| | ctactggggc cagggaaccc tggtcaccgt ctcctca | |
| 214 | QVQLVESGGG VVQPGRSLRL SCAASGFTFS | AA amino |
| | SYGMHWVRQT PGKGLEWVAF ISYDGNNKYY | acid |
| | ADSVKGRFTI SRDNSKNTLF LQMNSLRAED | sequence |
| | TAVYYCAKES ILAALFDYWG QGTLVTVSS | |
| 215 | ggattcacct tcagtagcta tggc | DNA |
| | | nucleotide |
| | | sequence |
| 216 | GFTFSSYG | AA amino |
| | | acid |
| | | sequence |
| 217 | atatcatatg atggaaataa taaa | DNA |
| | | nucleotide |
| | | sequence |
| 218 | ISYDGNNK | AA amino |
| | | acid |
| | | sequence |
| 219 | gcgaaagagt ccattttagc agccctcttt gactac | DNA |
| | | nucleotide |
| | | sequence |
| 220 | AKESILAALF DY | AA amino |
| | | acid |
| | | sequence |
| 221 | gacatcgtga tgacccagtc tccagactcc ctggctgtgt ctctgggcga | DNA |
| | gagggccacc atcaactgca agtccagcca gagtgtttta tacagctcca | nucleotide |
| | acaataagaa ctacttagct tggtaccagc agaaacctgg acagcctcct aagctgctca | sequence |
| | tttactgggc atctacccgg gaatccgggg tccctgaccg attcagtggc | |
| | agcgggtctg ggacagattt cactctcacc atcagcagcc tgcaggctga | |
| | agatgtggca ctttattact gtcaacaata ttataatact cccacttttg gccaggggac | |
| | caagctggag atcaaa | |
| 222 | DIVMTQSPDS LAVSLGERAT INCKSSQSVL | AA amino |
| | YSSNNKNYLA WYQQKPGQPP KLLIYWASTR | acid |
| | ESGVPDRFSG SGSGTDFTLT ISSLQAEDVA | sequence |
| | LYYCQQYYNT PTFGQGTKLE IK | |
| 223 | cagagtgttt tatacagctc caacaataag aactac | DNA |
| | | nucleotide |
| | | sequence |
| 224 | QSVLYSSNNK NY | AA amino |
| | | acid |
| | | sequence |
| 225 | tgggcatct | DNA |
| | | nucleotide |
| | | sequence |
| 226 | WAS | AA amino |
| | | acid |
| | | sequence |
| 227 | caacaatatt ataatactcc cact | DNA |
| | | nucleotide |
| | | sequence |
| 228 | QQYYNTPT | AA amino |
| | | acid |
| | | sequence |
| 229 | caggtgcagc tggtggagtc tgggggaggc gtggtccagc ctgggaggtc | DNA |
| | cctgagactc tcctgtgcag cctctggatt caccttcagt agctatggca tgcactgggt | nucleotide |
| | ccgccaggct ccagtcaagg ggctggagtg ggtggcattt atatcatatg | sequence |
| | atggaagtaa taaatactat gcagactccg tgaagggccg attcaccatc | |
| | tccagagaca attccaagaa cacgctgtat ctccaaatga acagcctgac | |
| | agctgaggac acggctgttt attactgtgc gaaagagtcc attttagcag ccctctttga | |
| | ctactggggc cagggaaccc tggtcaccgt ctcctca | |
| 230 | QVQLVESGGG VVQPGRSLRL SCAASGFTFS | AA amino |
| | SYGMHWVRQA PVKGLEWVAF ISYDGSNKYY | acid |
| | ADSVKGRFTI SRDNSKNTLY LQMNSLTAED | sequence |
| | TAVYYCAKES ILAALFDYWG QGTLVTVSS | |
| 231 | ggattcacct tcagtagcta tggc | DNA |
| | | nucleotide |
| | | sequence |
| 232 | GFTFSSYG | AA amino |
| | | acid |
| | | sequence |
| 233 | atatcatatg atggaagtaa taaa | DNA |
| | | nucleotide |
| | | sequence |
| 234 | ISYDGSNK | AA amino |
| | | acid |
| | | sequence |
| 235 | gcgaaagagt ccattttagc agccctcttt gactac | DNA |
| | | nucleotide |
| | | sequence |
| 236 | AKESILAALF DY | AA amino |
| | | acid |
| | | sequence |
| 237 | gacatcgtga tgacccagtc tccagactcc ctggctgtgt ctctgggcga | DNA |
| | gagggccacc atcaactgca agtccagcca gagtgtttta tacagttcca | nucleotide |
| | acaataagaa ctacttagct tggtaccagc agaaaccaag acagcctcct | sequence |
| | aagctgctca tttactgggc atctattcgg gaatccgggg tccctgaccg attcagtggc | |
| | agcgggtctg ggacagattt cattctcacc atcagcagcc tgcaggctga | |
| | agatgtggca gtttattact gtcagcaatt ttatagtatt cccacttttg gccaggggac | |
| | caagctggag atcaaa | |
| 238 | DIVMTQSPDS LAVSLGERAT INCKSSQSVL | AA amino |
| | YSSNNKNYLA WYQQKPRQPP KLLIYWASIR | acid |
| | ESGVPDRFSG SGSGTDFILT ISSLQAEDVA VYYCQQFYSI | sequence |
| | PTFGQGTKLE IK | |
| 239 | cagagtgttt tatacagttc caacaataag aactac | DNA |
| | | nucleotide |
| | | sequence |
| 240 | QSVLYSSNNK NY | AA amino |
| | | acid |
| | | sequence |
| 241 | tgggcatct | DNA |
| | | nucleotide |
| | | sequence |
| 242 | WAS | AA amino |
| | | acid |
| | | sequence |
| 243 | cagcaatttt atagtattcc cact | DNA |
| | | nucleotide |
| | | sequence |
| 244 | QQFYSIPT | AA amino |
| | | acid |
| | | sequence |
| 245 | caggtgcagc tggtggagtc tgggggaggc gtggtccagc ctgggaggtc | DNA |
| | cctgagactc tcctgtgcag cctctggatt cacctttagt agctatggca tgcactgggt | nucleotide |
| | ccgccaggct ccagtcaagg ggctggagtg ggtggcattt atatcaaatg | sequence |
| | ataaaagtaa taaatattat gcagactcct tgaagggccg attcaccatc tccagagaca | |
| | attccaagaa cacgctgtat ctgcaaatga acagcctgac agctgaagac acggctgttt | |
| | attactgtgc gaaagagtcc attttagcag ccctctttga ctattggggc cagggaaccc | |
| | tggtcaccgt ctcctca | |
| 246 | QVQLVESGGG VVQPGRSLRL SCAASGFTFS | AA amino |
| | SYGMHWVRQA PVKGLEWVAF ISNDKSNKYY | acid |
| | ADSLKGRFTI SRDNSKNTLY LQMNSLTAED | sequence |
| | TAVYYCAKES ILAALFDYWG QGTLVTVSS | |
| 247 | ggattcacct ttagtagcta tggc | DNA |
| | | nucleotide |
| | | sequence |
| 248 | GFTFSSYG | AA amino |
| | | acid |
| | | sequence |
| 249 | atatcaaatg ataaaagtaa taaa | DNA |
| | | nucleotide |
| | | sequence |
| 250 | ISNDKSNK | AA amino |
| | | acid |
| | | sequence |
| 251 | gcgaaagagt ccattttagc agccctcttt gactat | DNA |
| | | nucleotide |
| | | sequence |
| 252 | AKESILAALF DY | AA amino |
| | | acid |
| | | sequence |
| 253 | gacatcgtga tgacccagtc tccagactcc ctggctgtgt ctctgggcga | DNA |
| | gagggccacc atcaactgca agtccagcca gagtgtttta tacagctcca | nucleotide |
| | acaataagaa ctacttagct tggtaccagc agaaaccaag acagcctcct | sequence |
| | aagctactca tttactgggc atctattcgg gaatccgggg tccctgaccg attcagtggc | |
| | agcgggtctg ggacagattt cactctcacc atcagcagcc tgcaggctga | |
| | agatgtggca gtttattact gtcaacaatt ttatagtgtt cccacttttg gccaggggac | |
| | caagctggag atcaaa | |
| 254 | DIVMTQSPDS LAVSLGERAT INCKSSQSVL | AA amino |
| | YSSNNKNYLA WYQQKPRQPP KLLIYWASIR | acid |
| | ESGVPDRFSG SGSGTDFTLT ISSLQAEDVA VYYCQQFYSV | sequence |
| | PTFGQGTKLE IK | |
| 255 | cagagtgttt tatacagctc caacaataag aactac | DNA |
| | | nucleotide |
| | | sequence |
| 256 | QSVLYSSNNK NY | AA amino |
| | | acid |
| | | sequence |
| 257 | tgggcatct | DNA |
| | | nucleotide |
| | | sequence |
| 258 | WAS | AA amino |
| | | acid |
| | | sequence |
| 259 | caacaatttt atagtgttcc cact | DNA |
| | | nucleotide |
| | | sequence |
| 260 | QQFYSVPT | AA amino |
| | | acid |
| | | sequence |
| 261 | caggtgcagc tggtggagtc tgggggaggc gtggtccagc ctgggaggtc | DNA |
| | cctgagactc tcctgtgcag cctctggatt caccttcagt agctatggca tgcactgggt | nucleotide |
| | ccgccaggct ccagtcaagg ggctggagtg ggtggcattt atatcatttg atggaagtaa | sequence |
| | taaatactat acagactccg tgaagggccg attcaccatc tccagagaca attccaagaa | |
| | cacgctgtat ctccaaatga acagcctgac agctgaggac acggctattt attactgtgc | |
| | gaaagagtcc attttagcag ccctctttga ctactggggc cagggaaccc tggtcactgt | |
| | ctcctca | |
| 262 | QVQLVESGGG VVQPGRSLRL SCAASGFTFS | AA amino |
| | SYGMHWVRQA PVKGLEWVAF ISFDGSNKYY | acid |
| | TDSVKGRFTI SRDNSKNTLY LQMNSLTAED | sequence |
| | TAIYYCAKES ILAALFDYWG QGTLVTVSS | |
| 263 | ggattcacct tcagtagcta tggc | DNA |
| | | nucleotide |
| | | sequence |
| 264 | GFTFSSYG | AA amino |
| | | acid |
| | | sequence |
| 265 | atatcatttg atggaagtaa taaa | DNA |
| | | nucleotide |
| | | sequence |
| 266 | ISFDGSNK | AA amino |
| | | acid |
| | | sequence |
| 267 | gcgaaagagt ccattttagc agccctcttt gactac | DNA |
| | | nucleotide |
| | | sequence |
| 268 | AKESILAALF DY | AA amino |
| | | acid |
| | | sequence |
| 269 | gacatcgtga tgacccagtc tccagactcc ctggctgtgt ctctgggcga | DNA |
| | gagggccacc atcaactgca agtccagcca gagtgtttta tacagctcca | nucleotide |
| | acaataagaa ctacttagct tggtaccagc agaaaccaag acagcctcct | sequence |
| | aacctgctca tttactgggc atctattcgg gaatccgggg tccctgaccg attcagtggc | |
| | agcgggtctg ggacagattt cactctcacc atcagcagcc tgcaggctga | |
| | agatgtggca ttttattact gtcagcaatt ttatagtatt cccacttttg gccaggggac | |
| | caagctggag atcaaa | |
| 270 | DIVMTQSPDS LAVSLGERAT INCKSSQSVL | AA amino |
| | YSSNNKNYLA WYQQKPRQPP NLLIYWASIR | acid |
| | ESGVPDRFSG SGSGTDFTLT ISSLQAEDVA FYYCQQFYSI | sequence |
| | PTFGQGTKLE IK | |
| 271 | cagagtgttt tatacagctc caacaataag aactac | DNA |
| | | nucleotide |
| | | sequence |
| 272 | QSVLYSSNNK NY | AA amino |
| | | acid |
| | | sequence |
| 273 | tgggcatct | DNA |
| | | nucleotide |
| | | sequence |
| 274 | WAS | AA amino |
| | | acid |
| | | sequence |
| 275 | cagcaatttt atagtattcc cact | DNA |
| | | nucleotide |
| | | sequence |
| 276 | QQFYSIPT | AA amino |
| | | acid |
| | | sequence |
| 277 | tcctgtgcag cctctggatt cacctttagg acctatggca tgcactgggt ccgccaggct | DNA |
| | ccagtcaagg ggctggagtg ggtggcattt atatcaaagg atggaagtga taaatactat | nucleotide |
| | gtagactccg tgaagggccg attcaccatc tccagagaca attccaagaa cacgctgttt | sequence |
| | ctgcaaatga acagcctgac agctgaggac acggctgttt attattgtgc gaaagagtcc | |
| | attttagcag ccctctttga ctactggggc cagggaaccc tggtcactgt ctcctca | |
| 278 | QVQLVESGGG VVQPGRSLRL SCAASGFTFR | AA amino |
| | TYGMHWVRQA PVKGLEWVAF ISKDGSDKYY | acid |
| | VDSVKGRFTI SRDNSKNTLF LQMNSLTAED | sequence |
| | TAVYYCAKES ILAALFDYWG QGTLVTVSS | |
| 279 | ggattcacct ttaggaccta tggc | DNA |
| | | nucleotide |
| | | sequence |
| 280 | GFTFRTYG | AA amino |
| | | acid |
| | | sequence |
| 281 | atatcaaagg atggaagtga taaa | DNA |
| | | nucleotide |
| | | sequence |
| 282 | ISKDGSDK | AA amino |
| | | acid |
| | | sequence |
| 283 | gcgaaagagt ccattttagc agccctcttt gactac | DNA |
| | | nucleotide |
| | | sequence |
| 284 | AKESILAALF DY | AA amino |
| | | acid |
| | | sequence |
| 285 | atcaactgca agtccagcca gagtgtttta tacagctcca acaataagaa ctacttagct | DNA |
| | tggtaccagc agaaaccaag acagcctcct aaactcctca tttactgggc atctaatcgg | nucleotide |
| | gaatccgggg tccctgaccg attcagtggc agcgggtctg ggacagattt | sequence |
| | cactctcacc atcagcagcc tgcaggctga agatgtggca gtttattact gtcagcaatt | |
| | ttatagtgtt cccacttttg gccaggggac caagctggag atcaaa | |
| 286 | DIVMTQSPDS LAVSLGERAT INCKSSQSVL | AA amino |
| | YSSNNKNYLA WYQQKPRQPP KLLIYWASNR | acid |
| | ESGVPDRFSG SGSGTDFTLT ISSLQAEDVA VYYCQQFYSV | sequence |
| | PTFGQGTKLE IK | |
| 287 | cagagtgttt tatacagctc caacaataag aactac | DNA |
| | | nucleotide |
| | | sequence |
| 288 | QSVLYSSNNK NY | AA amino |
| | | acid |
| | | sequence |
| 289 | tgggcatct | DNA |
| | | nucleotide |
| | | sequence |
| 290 | WAS | AA amino |
| | | acid |
| | | sequence |
| 291 | cagcaatttt atagtgttcc cact | DNA |
| | | nucleotide |
| | | sequence |
| 292 | QQFYSVPT | AA amino |
| | | acid |
| | | sequence |
| 293 | caggtgcagc tggtggagtc tgggggaggc gtggtccagc ctgggaggtc | DNA |
| | cctgagactc tcctgtgcag cctctggatt cacctttagt agctatggca tgcactgggt | nucleotide |
| | ccgccaggct ccagtcaagg ggctggagtg ggtggcattt atatcaaatg | sequence |
| | ataaaagtaa taaatactat gcagactccg tgaagggccg attcaccatc tccagagaca | |
| | attccaagaa cacgctgtat ctgcaaatga acagcctgac agctgaggac acggctgttt | |
| | attactgtgc gaaagagtcc attttagcag ccctctttga ctcctggggc cagggaaccc | |
| | tggtcaccgt ctcctca | |
| 294 | QVQLVESGGG VVQPGRSLRL SCAASGFTFS | AA amino |
| | SYGMHWVRQA PVKGLEWVAF ISNDKSNKYY | acid |
| | ADSVKGRFTI SRDNSKNTLY LQMNSLTAED | sequence |
| | TAVYYCAKES ILAALFDSWG QGTLVTVSS | |
| 295 | ggattcacct ttagtagcta tggc | DNA |
| | | nucleotide |
| | | sequence |
| 296 | GFTFSSYG | AA amino |
| | | acid |
| | | sequence |
| 297 | atatcaaatg ataaaagtaa taaa | DNA |
| | | nucleotide |
| | | sequence |
| 298 | ISNDKSNK | AA amino |
| | | acid |
| | | sequence |
| 299 | gcgaaagagt ccattttagc agccctcttt gactcc | DNA |
| | | nucleotide |
| | | sequence |
| 300 | AKESILAALF DS | AA amino |
| | | acid |
| | | sequence |
| 301 | gacatcgtga tgacccagtc tccagactcc ctggctgtgt ctctgggcga | DNA |
| | gagggccacc atcaactgca agtccagcca gagtgtttta tacagctcca | nucleotide |
| | acaataagaa ctacttagct tggtaccagc agaaaccaag acagcctcct | sequence |
| | aagctgctca tttactgggc atctattcgg gaatccgggg tccctgaccg attcagtggc | |
| | agcgggtctg gggcagattt cactctcacc atcagcagcc tgcaggctgc | |
| | agatgtggca gtttattact gtcagcaatt ttatagtgtt cccacttttg gccaggggac | |
| | caagctggag atcaaa | |
| 302 | DIVMTQSPDS LAVSLGERAT INCKSSQSVL | AA amino |
| | YSSNNKNYLA WYQQKPRQPP KLLIYWASIR | acid |
| | ESGVPDRFSG SGSGADFTLT ISSLQAADVA | sequence |
| | VYYCQQFYSV PTFGQGTKLE IK | |
| 303 | cagagtgttt tatacagctc caacaataag aactac | DNA |
| | | nucleotide |
| | | sequence |
| 304 | QSVLYSSNNK NY | AA amino |
| | | acid |
| | | sequence |
| 305 | tgggcatct | DNA |
| | | nucleotide |
| | | sequence |
| 306 | WAS | AA amino |
| | | acid |
| | | sequence |
| 307 | cagcaatttt atagtgttcc cact | DNA |
| | | nucleotide |
| | | sequence |
| 308 | QQFYSVPT | AA amino |
| | | acid |
| | | sequence |

What is claimed is:

1. A method for treating a condition or disease associated with, or characterized in part by hyperammonemia, or at least one symptom or complication associated with the condition or disease in a subject in need thereof, the method comprising administering to the subject, a therapeutically effective amount of a composition comprising a glucagon signaling pathway antagonist such that,
plasma ammonia levels in the subject are lowered, or
the condition or disease is mediated, or
at least one symptom or complication associated with the condition or disease is alleviated or reduced in severity;
wherein the glucagon signaling pathway antagonist is an isolated human monoclonal antibody or antigen-binding fragment thereof that specifically binds glucagon receptor, and wherein the isolated human monoclonal antibody or antigen-binding fragment thereof comprises a heavy chain complementarity determining region (HCDR)1, HCDR2, and HCDR3 within a heavy chain variable region (HCVR) amino acid sequence as set forth in SEQ ID NO: 34; and a light chain complementarity determining region (LCDR)1, LCDR2, and LCDR3 within a light chain variable region (LCVR) amino acid sequence as set forth in SEQ ID NO: 42.

2. The method of claim 1, wherein the hyperammonemia is acquired.

3. The method of claim 1, wherein the hyperammonemia is congenital hyperammonemia.

4. The method of claim 1, wherein the congenital hyperammonemia is caused by
(i) a defect in one or more urea cycle enzymes selected from the group consisting of carbamyl phosphate synthetase (CPS1), N-acetylglutamate synthetase (NAGS), ornithine transcarbamylase (OTC), argininosuccinic acid synthetase (ASS), argininosuccinate lyase (ASL), and arginase (AR1);
(ii) a defect in one or more urea cycle transporters, wherein the urea cycle transporters are ornithine translocase (ORNT1) or citrin;
(iii) methylmalonic aciduria, propionic aciduria, and/or isovaleric aciduria;
(iv) medium-chain acyl-CoA dehydrogenase deficiency, multiple acyl-CoA dehydrogenase deficiency, carnitine palmitoyltransferase II deficiency, carnitine-acylcarnitine translocase, lysinuric protein intolerance, pyrroline-5-carboxylate synthetase deficiency, pyruvate carboxylase deficiency, ornithine aminotransferase deficiency, carbonic anhydrase Va deficiency, hyperinsulinism-hyperammonemia syndrome, mitochondrial disorders and/or glutamine synthetase deficiency;
(v) acute or chronic liver failure and complications thereof;
(vi) treatment with a therapeutic agent, L-asparaginase, pegaspargase, 5-pentanoic acid, valproic acid, a corticosteroid and/or a cyclophosphamide;
(vii) herpes simplex infection, hepatitis B infection and/or infection with urease-producing organisms; and/or
(viii) total parenteral nutrition with relative arginine deficiency, L-asparaginase treatment, nutritional carnitine deficiency, cystoscopy with glycine-containing solutions, post-lung/bone marrow transplantation, vascular malformations and/or transient hyperammonemia in a subject which is a newborn.

5. The method of claim 1, wherein the isolated human monoclonal antibody or antigen-binding fragment thereof that specifically binds glucagon receptor comprises an HCDR1 amino acid sequence of SEQ ID NO: 36, an HCDR2 amino acid sequence of SEQ ID NO: 38, an HCDR3 amino acid sequence of SEQ ID NO: 40, an LCDR1 amino acid sequence of SEQ ID NO: 44, an LCDR2 amino acid sequence of SEQ ID NO: 46, and an LCDR3 amino acid sequence of SEQ ID NO: 48.

6. The method of claim 51, wherein the isolated human monoclonal antibody or antigen-binding fragment thereof that specifically binds glucagon receptor comprises an HCVR amino acid sequence of SEQ ID NO: 34 and an LCVR amino acid sequence of SEQ ID NO: 42.

7. The method of claim 1, wherein the composition is administered to the subject in combination with at least one additional therapeutic agent or supplement.

8. The method of claim 1, wherein the glucagon signaling pathway antagonist is administered concomitantly with:
(i) one or more amino acid formulas selected from Cyclinex, EAA, UCD-I, UCD-II, and individual branched chain amino acids;
(ii) antioxidants and/or electrolytes;
(iii) L-citrulline and/or L-arginine free base;
(iv) hemodialysis and/or continuous renal replacement; and/or
(v) a non-absorbable antibiotic, rifaximin, lactulose, sodium phenylbutyrate, sodium benzoate, sodium phenylacetate, glycerol phenylbutyrate, carbamyl glutamate, a second GCG inhibitor, and/or a second GCGR antagonist.

9. A method of treating a subject with a urea cycle disorder, wherein the subject exhibits elevated levels of ammonia, the method comprising administering to the subject a therapeutically effective amount of a composition comprising a glucagon signaling pathway antagonist,
wherein the glucagon signaling pathway antagonist is an isolated human monoclonal antibody or antigen-binding fragment thereof that specifically binds glucagon receptor, and wherein the isolated human monoclonal antibody or antigen-binding fragment thereof comprises an HCDR1, HCDR2, and HCDR3 within an HCVR amino acid sequence as set forth in SEQ ID NO: 34; and an LCDR1, LCDR2, and LCDR3 within an LCVR amino acid sequence as set forth in SEQ ID NO: 42.

10. The method of claim 9, wherein the subject having a urea cycle disorder suffers from
(i) one or more defects in a urea cycle enzyme selected from the group consisting of carbamyl phosphate synthetase (CPS1), N-acetylglutamate synthetase (NAGS), ornithine transcarbamylase (OTC), argininosuccinic acid synthetase (ASS), argininosuccinate lyase (ASL), and arginase (AR1); and/or
(ii) one or more defects in a urea cycle transporter selected from ornithine translocase (ORNT1) and citrin.

11. The method of claim 9, wherein the isolated human monoclonal antibody or antigen-binding fragment thereof that specifically binds glucagon receptor comprises an HCDR1 amino acid sequence of SEQ ID NO: 36, an HCDR2 amino acid sequence of SEQ ID NO: 38, an HCDR3 amino acid sequence of SEQ ID NO: 40, an LCDR1 amino acid sequence of SEQ ID NO: 44, an LCDR2 amino acid sequence of SEQ ID NO: 46, and an LCDR3 amino acid sequence of SEQ ID NO: 48.

12. The method of claim 9, wherein the composition is administered to the subject in combination with at least one additional therapeutic agent or supplement.

13. The method of claim 9, wherein the glucagon signaling pathway antagonist is administered concomitantly with:
(i) one or more amino acid formulas selected from the group consisting of Cyclinex, EAA, UCD-I, UCD-II, and individual branched chain amino acids;
(ii) antioxidants and/or electrolytes;
(iii) L-citrulline and/or L-arginine free base;
(iv) hemodialysis and/or continuous renal replacement; and/or
(v) a non-absorbable antibiotic, rifaximin, lactulose, sodium phenylbutyrate, sodium benzoate, sodium phenylacetate, glycerol phenylbutyrate, carbamyl glutamate, a second GCG inhibitor, and/or a second GCGR antagonist.

14. A method of reducing the amount and/or dosage of sodium phenylbutyrate or sodium benzoate necessary to treat a subject with hyperammonemia, the method comprising administering to the subject a therapeutically effective amount of a composition comprising a glucagon signaling pathway antagonist;
wherein the glucagon signaling pathway antagonist is an isolated human monoclonal antibody or antigen-binding fragment thereof that specifically binds glucagon receptor, and wherein the isolated human monoclonal antibody or antigen-binding fragment thereof comprises an HCDR1, HCDR2, and HCDR3 within an HCVR amino acid sequence as set forth in SEQ ID NO: 34; and an LCDR1, LCDR2, and LCDR3 within an LCVR amino acid sequence as set forth in SEQ ID NO: 42.

15. The method of claim 14, wherein the glucagon signaling pathway antagonist is administered concomitantly with sodium phenylbutyrate and/or sodium benzoate.

16. The method of claim 14 wherein the isolated human monoclonal antibody or antigen-binding fragment thereof that specifically binds glucagon receptor comprises an HCDR1 amino acid sequence of SEQ ID NO: 36, an HCDR2 amino acid sequence of SEQ ID NO: 38, an HCDR3 amino acid sequence of SEQ ID NO: 40, an LCDR1 amino acid sequence of SEQ ID NO: 44, an LCDR2 amino acid sequence of SEQ ID NO: 46, and an LCDR3 amino acid sequence of SEQ ID NO: 48.

17. The method of claim 14, wherein the isolated human monoclonal antibody or antigen-binding fragment thereof that specifically binds glucagon receptor comprises an HCVR amino acid sequence of SEQ ID NO: 34 and an LCVR amino acid sequence of SEQ ID NO: 42.

18. A method for lowering blood ammonia levels, or for treating a condition or disease associated with, or characterized in part by hyperammonemia, or at least one symptom or complication associated with the condition or disease in a subject in need thereof, the method comprising administering, to the subject, a therapeutically effective amount of a composition comprising glucagon signaling pathway antagonist, such that blood ammonia levels are lowered or that the condition or disease is mediated, or at least one symptom or complication associated with the condition or disease is alleviated or reduced in severity;
wherein the glucagon signaling pathway antagonist is an isolated human monoclonal antibody or antigen-binding fragment thereof that specifically binds glucagon receptor, and wherein the isolated human monoclonal antibody or antigen-binding fragment thereof comprises an HCDR1,HCDR2, and HCDR3 within an HCVR amino acid sequence as set forth in SEQ ID NO: 34; and an LCDR1, LCDR2, and LCDR3 within an LCVR amino acid sequence as set forth in SEQ ID NO: 42.

19. The method of claim 18, wherein the isolated human monoclonal antibody or antigen-binding fragment thereof that specifically binds glucagon receptor comprises an HCDR1 amino acid sequence of SEQ ID NO: 36, an HCDR2 amino acid sequence of SEQ ID NO: 38, an HCDR3 amino acid sequence of SEQ ID NO: 40, an LCDR1 amino acid sequence of SEQ ID NO: 44, an LCDR2 amino acid sequence of SEQ ID NO: 46, and an LCDR3 amino acid sequence of SEQ ID NO: 48.

20. A method for reducing excessive weight loss and/or lowering blood glucose in a subject with a urea cycle disorder, comprising administering to the subject a therapeutically effective amount of a composition comprising a glucagon signaling pathway antagonist;
wherein the glucagon signaling pathway antagonist is an isolated human monoclonal antibody or antigen-binding fragment thereof that specifically binds glucagon receptor, and wherein the isolated human monoclonal antibody or antigen-binding fragment thereof comprises an HCDR1, HCDR2, and HCDR3 within an HCVR amino acid sequence as set forth in SEQ ID NO: 34; and an LCDR1, LCDR2, and LCDR3 within an LCVR amino acid sequence as set forth in SEQ ID NO: 42.

21. The method of claim 20 wherein the subject is on a high protein diet.

22. The method of claim 20, wherein the isolated human monoclonal antibody or antigen-binding fragment thereof that specifically binds glucagon receptor comprises an HCDR1 amino acid sequence of SEQ ID NO: 36, an HCDR2 amino acid sequence of SEQ ID NO: 38, an HCDR3 amino acid sequence of SEQ ID NO: 40, an LCDR1 amino acid sequence of SEQ ID NO: 44, an LCDR2 amino acid sequence of SEQ ID NO: 46, and an LCDR3 amino acid sequence of SEQ ID NO: 48.

23. The method of claim 20, wherein the isolated human monoclonal antibody or antigen-binding fragment thereof that specifically binds glucagon receptor comprises an HCVR amino acid sequence of SEQ ID NO: 34 and an LCVR amino acid sequence of SEQ ID NO: 42.

24. The method of claim 9, wherein the isolated human monoclonal antibody or antigen-binding fragment thereof that specifically binds glucagon receptor comprises an HCVR amino acid sequence of SEQ ID NO: 34 and an LCVR amino acid sequence of SEQ ID NO: 42.

25. The method of claim 18, wherein the isolated human monoclonal antibody or antigen-binding fragment thereof that specifically binds glucagon receptor comprises an HCVR amino acid sequence of SEQ ID NO: 34 and an LCVR amino acid sequence of SEQ ID NO: 42.
